SEQUENCE LISTING (33 sequences)

SEQ ID NO: 1 - DNA, 325 bp, Artificial Sequence
  Synthetic Construct
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaacccagaagttagttaattatttgtgtagtcaaagtgccttgactacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattc

SEQ ID NO: 2 - DNA, 663 bp, Artificial Sequence
  Synthetic Construct
aattggcagtaaagtggcagtttttgatacctaaaatgagatattatgatagtgtaggatattgactatcttactgcgtttcccttatcgcaattaggaataaaggatctatgtgggttggctgattatagccaatccttttttaattttaaaaagcgtatagcgcgagagttggtggtaaatgaaatgaacgaaaaacaaaagagattcgcagatgaatatataatgaatggatgtaatggtaaaaaagcagcaatttcagcaggttatagtaagaaaacagcagagtctttagcaagtcgattgttaagaaatgttaatgtttcggaatatattaaagaacgattagaacagatacaagaagagcgtttaatgagcattacagaagctttagcgttatctgcttctattgctagaggagaacctcaagaggcttacagtaagaaatatgaccatttaaacgatgaagtggaaaaagaggttacttacacaatcacaccaacttttgaagagcgtcagagatctattgaccacatactaaaagttcatggtgcgtatatcgacaaaaaagaaattactcagaagaatattgagattaatattggtgagtacgatgacgaaagttaaattaaactttaacaaaccatctaatgttttcaacag

SEQ ID NO: 3 - DNA, 227 bp, Artificial Sequence
  Synthetic Construct
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtc

SEQ ID NO: 4 - DNA, 55 bp, Artificial Sequence
  Synthetic Construct
tgcattaaaaccgccccgcaaagcgggcgggcgaggcggggaaagcaccgcgcgc

SEQ ID NO: 5 - DNA, 735 bp, Artificial Sequence
  Synthetic Construct
atgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatga

SEQ ID NO: 6 - DNA, 573 bp, Artificial Sequence
  Synthetic Construct
atggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaa

SEQ ID NO: 7 - DNA, 501 bp, Artificial Sequence
  Synthetic Construct
atgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatga

SEQ ID NO: 8 - DNA, 9181 bp, Artificial Sequence
  Synthetic Construct
tcagtcgttgtcagtgtccagtgagtagtttttaaagcggatgacctcctgaccgagccagccgtttatctcgcggatcctgtcctgtaacgggataagctcattgcggacaaagacctttgccactttctcaatatcacccagcgacccgacgttctccggcttgccacccatcaactgaaaggggatgcggtgcgcgtccagcaggtcagcggcgctggcttttttgatattaaaaaaatcgtccttcgtcgccacttcactgagggggataattttaatgccgtcggctttcccctgtggggcatagagaaacaggtttttaaagttgttgcggcctttcgacttgaccatgttttcgcgaagcatttcgatatcgttgcgatcctgcacggcatcggtgacatacatgatgtatccggcatgtgcgccattttcgtaatacttgcggcggaacaacgtggccgactcattcagccaggcagagttaagggcgctgagatattccggcaggccgtacagctcctgattaatatccggctccagcaggtgaaacacggagccgggcgcgaaggctgtcggctcgttgaaggacggcacccaccagtaaacatcctcttccacgccacggcgggtatattttgccggtgaggtttccagtctgatgaccttaccggtggtgctgtaacgcttttccagaaacgcattaccgaacaccagaaaatccagcacaaagcggctgaaatcctgctgggaaagccatggatgcgggataaatgtcgaggccagaatattgcgtttgacgtaaatcggcgagctgtgatgcacggcagcccgcaggctttttgccagaccggtaaagctgaccggtggctcataccatctgccgttactgatgcactcgacgtaatccagaatgtcacggcggtcgagtaccggcaccggctcaccaaaggtgaatgcctccattttcgggccgctggcggtcattgtttttgccgcaggttgcggtgttttcccttttttcttgctcatcagtaaaactccagaatggtggatgtcagcggggtgctgataccggcggtgagtggctcatttaacagggcgtgcatggtcgcccaggcgaggtcggcgtggctggcttcctcgctgcggctggcctcataggtggcgctgcgtccgctgctggtcatggtcttgcggatagccataaacgagctggtgatgtcggtggcgctgacgtcgtattccagacagccacggcggataacgtcttttgccttgagcaccattgcggttttcatttccggcgtgtagcggatatcacgcgcggcgggatagaacgagcgcacgagctggaacacgccgacaccgaggccggtggcatcaataccgatgtattcgacgttgtatttttcggtgagtttgcggatggattccgcctgggtggcaaagtccatgcctttccactggtgacgctcaagtattctgaatttgccaccggccaccaccggcggtgccagtaccacgcatccggcgctgtcgccacggtgtgacgggtcgtaaccaatccataccgggcgggagccgaacggattggcggcaaacggcgcatagtcttcccattcttccagcgtgtcgaccatgcagcgttgcagctcctcgaacgggaacaccgatgccttgtcgtcaacaaattcacacatgaacaggtttttaaaatcgtcggcgctgttttcgcgtttgagctgctcaatgtcgaacagcgtgcagccgcctttcagggcgtcctcaatggtgacaatctgtcgccactggccgtccgcacagagaagcccaccggcaagggcgttatgactgacgtcgatttccacgcgttcggcggcgctggcgcgtccccggttaaacagttcacccgaccagaacgggtaggcgtcgtgcgccagcgtggacggggtggagaaataggtcgagcgcaggtgactctgtgaggccatacctgatgccaccttacgcagtacctgaaaattcgggatccagaaaatctcatcgacgtacaggtcgccgttatgactctgcgcggtgttggagttggtgccgagaaaaatcagttttgcgccgttattgcccaggacaatcgggtcaccggtcaggtcaacgtcaaccagacgggcaaaggcgatgatgtattcgcggaacacatacgcctgtgttttactggccgacagaaaaatctggttatgaccggttttcagggcacgcagcagcgcctcgcgggaaaaataaaacgtcgcgccaatctggcgggatttcaggatatcgcggatgcggtgctcaagcccggcacgataccagtgcagctgatagtcgaaagactgctcaaagaaaatctgctccagcttttcgatggcctcgtcactgaaaaaattctttttcggtttgcgccgtccgcctttgttacggttagcgacgttcggattaaggtctgcctcgttgccggtctggctgtagcggttgacccgtgccagtcgttcaatctggcgtcccagcaggtcaatttccttgaagtcaccgccggttttctgtggtttgatgatgagctgggtcagccgcgcttccagactcatttcgacacggctgatgggggcaacgctgtcccagccgtcgcgctgtttccagctctgcactgtcgggcgtttcatctgcaacatggcggcaatctgcggcacggaaaacccctgccagtacagcagcgccgcctgacgacgcgggtcgtgtaaaagagtggtgtctgtggtgatggtcatgaatacctcgccgtgatgaatacacggcaaggctactgagtcgcgccccgcgattcgctaaggtgctgttgtgtcagtgataagccatccgggactgatggcggaggatgcgcatcgtcgggaaactgatgccgacatgtgactcctctaatcactattcaggactcctgacaatggcaaaaaaagtctcaaaattctttcgtatcggcgttgagggtgacacctgtgacgggcgtgtcatcagtgcgcaggatattcaggaaatggccgaaacctttgacccgcgtgtctatggttgccgcattaacctggaacatctgcgcggcatcctgcctgacggtatttttaagcgttatggcgatgtggccgaactgaaggccgaaaagattgacgatgattcggcgctgaaaggcaaatgggcgctgtttgcgaaaatcaccccgaccgatgaccttatcgcgatgaacaaggccgcgcagaaggtctacacctcaatggaaattcagccgaactttgccaacaccggcaaatgttatctggtgggtctggccgtcaccgatgacccggcaagcctcggcacggaatacctggaattctgccgcacggcaaaacacaaccccctgaaccgcttcaaattaagccctgaaaacctgatttcagtggcaacgcctgttgagctggaatttgaagacctgcctgaaaccgtgttcaccgccctgaccgaaaaggtgaagtccatttttggccgcaaacaggccagcgatgatgcccgtctgaatgacgtgcatgaagcggtgaccgctgttgctgaacatgtgcaggaaaaactgagcgccactgagcagcgcctcgctgagatggaaaccgccttttctgcacttaagcaggaggtgactgacagggcggatgaaaccagccaggcattcacccgcctgaaaaacagtctcgaccacaccgaaagtctgacccagcagcgccgcagcaaagccaccggcggtggcggtgacgccctgatgacgaactgctgaccggcgtcagtcagtccgggaaaaccttcacgattaacccttaatttcaggaaaaactatgcgccaggaaacccgctttaaatttaatgcctacctgtcccgtgttgccgaactgaacggcatcgacgccggtgatgtgtcgaaaaaattcaccgttgaaccgtcggtcacccagaccctgatgaacaccatgcaggagtcctctgactttctgacccgcatcaatattgtgccggtcagcgaaatgaaaggggaaaaaattggtatcggtgtcaccggctccatcgccagcactaccgacactgccggtggtaccgagcgtcagccgaaggacttctcgaagctggcgtcaaacaagtacgaatgcgaccagattaacttcgatttttatatccgctacaaaacgctggacctgtgggcgcgttatcaggatttccagctccgtatccgtaacgccattatcaaacgccagtcccttgatttcatcatggccggttttaacggcgtgaagcgtgccgaaacctctgaccgcagcagcaatccgatgttgcaggatgtggcggtcggctggctgcagaaataccgcaatgaagcaccggcgcgcgtgatgagcaaggtcactgacgaggaaggccgcaccacctctgaggttatccgcgtgggtaagggcggtgattatgccagccttgatgcactggtgatggatgcgaccaacaacctgattgaaccgtggtatcaggaagaccctgaccttgtggtgattgtggggcgtcagctactggcggacaagtatttccccatcgtcaacaaggagcaggacaacagcgaaatgctggccgctgacgtcatcatcagccagaaacgcatcggtaacctaccagcggtacgcgtcccgtacttcccggcggatgcgatgctcatcacgaagctggaaaacctgtccatctactacatggatgacagccatcgccgcgtgattgaggaaaacccgaaactcgaccgcgtggagaactacgagtcaatgaacattgattacgtggtggaagactacgccgccggttgtctggtggaaaaaatcaaggtcggtgacttctccacaccggctaaggcgaccgcagagccgggagcgtaaccgatgacgagtcccgcacagcgccacatgatgcgggtctcggcagcgatgaccgcgcagcgggaagccgccccgctgcgacatgcaactgtctatgagcagatgctggttaagctcgccgcagaccagcgcacactgaaagcgatttactcaaaagagctgaaggccgcaaaaaaacgcgaactgctgccgttctggttgccgtgggtgaacggcgtgctggagctgggcaaaggtgcacaggatgacattctgatgacggtcatgctgtggcgtctggataccggcgatattgccggtgcgctggagattgcccgttatgccctgaagtacggtctgaccatgccgggtaaacaccgccgtaccccgccgtacatgttcaccgaggaggtagcgcttgcggccatgcgcgctcacgctgccggtgagtctgtggatacccgcctgctgacggagacccttgaactgaccgccacggctgacatgcctgatgaagtgcgcgcaaagctgcacaaaatcaccggtctgtttctgcgtgacggtggtgatgccgccggtgcgctggcgcacctgcaacgtgcgacacagctcgactgtcaggcaggcgtcaaaaaagagattgaacgactggagcgggagctgaaaccgaagccggagccgcagcccaaagcggccacccgcgccccgcgtaagacccggagcgtgacaccggcaaaacgtggacgcccgaaaaagaaagccagttaacaaccgaatgcgccccgcgccagggcggcacgccggtcagtgacggtgaatcacctgacactgcaccggcgtccaccgcccgacttttcagaggtagtcatgatgacgctgattattccgcgaaaggaggctcccgtgtccggtgagggtacggtggtcatcccgcaaccggcaggcgacgagccggtgattaaaaacacgttcttttttcccgatatcgacccgaagcgcgtccgggaacgtatgcgccttgagcagaccgtcgcccccgcccgtctgcgtgaggccatcaagtcaggcatggctgaaacgaatgcggagctgtacgagtaccgcgaacagaaaattgccgccggttttacgcgtctggctgacgtcccggcggacgatatcgacggtgaaagcatcaaggttttttactacgagcgcgccgtgtgtgcgatggcgaccgcgtcgctttatgagcgttatcgcggtgtggatgccagtgcgaaaggcgacaagaaggctgacagcattgacagcaccattgatgagctgtggcgggatatgcgctgggcggtggcgcgcatccagggcaagccgcgctgcatcgtgagtcaaatctgatgaagacctttgcgctacagggcgacacgctcgacgccatttgtgtccgctattacgggcgcactgagggcgtggttgagaccgtgctcgccgcaaatccgggactggctgaactgggggcggtgctgccacacggcaccgccgtcgaactgcccgacgttcagaccgcgcccgtggctgaaactgtcaatctgtgggagtaacgcatgacagcagaagaaaaaagcgtcctgtcgcttttcatgattggggtgctgattgttgtcggcaaggtgcttgccggtggtgaacctatcaccccgcgtctgtttatcgggcgcatgttgctcggtggttttgtctcgatggttgccggtgttgttctggtgcagtttcctgacctgtcactgccagcggtgtgcggcatcggctccatgctgggtatcgccggttatcaggtgattgagattgccattcagcgccgctttaagggcagggggaaacagtaatgccggtaattaacacgcatcagaatatcgccgcctttctcgacatgctggccgtgtccgaagggacggcgaatcatccactgacgaaaaaccggggctatgacgtgatagtcaccggactggacgggaagccggaaattttcaccgactacagtgaccacccgttcgcacatggccgaccggcgaaggtgtttaaccgtcgcggtgaaaaatccacggcctccggtcgctatcagcagctttacctgttctggccgcattaccgcaaacagcttgccctgccggatttcagtccgttgtcacaggacagactcgccattcagttgatccgcgaacgcggagcactggatgacatccgggcgggacgcattgagcgcgccatttcacgctgtcgcaatatctgggcgtccctgccgggtgccggttacggtcagcgtgagcattcactggaaaaactggtcaccgtctggcgtaccgctggcggcgtaccggcttaaacggagtaaataccatgaagaaattatccctttcactgatgctgaacgtgtcgctggcgctgatgctggcactgtccctgatttacccgcagagcgtggccgtcaattttgtcgctgcctgggcgattctggcgacggttatctgtgtggttgccggtggtgtgggcgtgtatgccactgagtatgtgctggaacgctacgggcgggagctgccgccggaatcgctggccgtgaagattgtcacgtcgctgtttttgcagccggtgccgtggcgcagacgggcggcggctctggtagtggtggtggcgacgtttatctcgctggtcgctgccgggtggatttttaccgcgctgatttatcttgtggtgtcgctgtttttccggctgatacgtaaagcctgtcgtcagcgtcttgaggggcgggaaccatgtcaaggctgatgattgtgctggtcgtgttgttatcgctggcggtggccggtctgtttctggtgaaacacaaaaatgccagcctgcgcgcctcgctggacagggcgaacaacgtcgccagcggtcagcagacgaccatcaccatgctgaaaaatcagcttcatgttgcgctcaccagggcagataaaaacgagctggcgcaggtggcactgcgtcaggaactggagaacgccgcgaaacgtgaagcacagcgcgagaaaaccatcacgaggttacttaatgagaacgaagattttcgccgctggtacggtgctgacctgcctgatgctgtgcgccggttgcaccagcgccccgcctgcaccgacgccagtgattgtccccaacgcatgcccgaaagtgagcctttgcccgatgccgggcagtgacccgcagacgaacggcgatttaagtgccgatatccggcagcttgagaacgcgctggcacgctgtgccagccaggtaaaaatgattaaacactgtcaggacgaaaacgatgctcaaacccgacagcctgcgcagggcgctgactgatgccgtcacggtgctgaaaactaaccccgatatgctgcggatattcgtggataacgggagtattgcctccacactggcggcgtcgctgtcattcgaaaagcgttacacgctcaatgtgattgtgaccgactttaccggtgattttgacctgctcattgtgccggtgctggcgtggctgcgggaaaatcagcccgacatcatgaccaccgacgaaggccagaaaaagggcttcacgttttatgcagacatcaacaatgacagcagctttgatatcagtatcagcctgatgctgaccgagcgcacgctggtcagtgaggtggacggcgcactgcatgtgaagaatatctcggaacccccgccgccggagccggtcacccgcccgatggagctgtatatcaatggcgaactggtgagtaagtgggatgaatgagtttaagcgttttgaagaccggctgaccggactgattgaatcgctgtcaccgtcagggcgtcggcgactgagtgccgaactggcgaaacgtctgcggcagagtcagcagcgtcgggtgatggcacagaaagccccggacggcacaccctacgcgccacgccagcagcagagcgtcagaaaaaagaccggtcgcgttaagcgaaaaatgtttgcgaaacttattaccagtcgttttttgcatatccgtgccagcccggagcaggcatcaatggaattttacggcgggaagtcgccgaaaatcgccagtgtgcatcagtttggtctgtcggaagaaaaccggaaagacggtaagaaaattgattatccggcgcgtcccctgctcggctttaccggtgaggatgtgcagatgattgaagagattatcctggctcaccttgagcgttag

SEQ ID NO: 9 - DNA, 4967 bp, Artificial Sequence
  Synthetic Construct
atgaacactctcgcaaatattcaggaactcgcgcgcgcactgcgcaacatgattcgcactggcattatcgtcgaaaccgaccttaacgccggtcgctgccgcgtgcagaccggcggcatgtgcaccgactggcttcagtggctgacccatcgcgcaggacgttcgcgcacatggtgggcaccttccgtgggggaacaggtgctgattctggccgtgggtggtgaactcgacacggcgttcgttctgccggggatttattccggcgataacccctcgccgtctgtgtcggcggatgccctgcatatccgtttccctgacggggcggtgattgaatatgaacccgaaaccagtgcactcacggtaagcggaattaaaacggccagcgtgacggcttccggttctgttactgccacggtgccggtggtcatggtgaaagcatcaacccgcgtcaccctggacaccccggaggtggtctgcaccaacaggctgattaccggcacgctggaagtgcagaaaggcgggacgatgcgcggcaacattgaacacaccggcggtgaactctcatcaaacggtaaggtactgcatacccataaacaccccggcgacagcggcggcacaaccgggagtcctttatgacagcgcgttatctcggaatgaatcgcagtgatggcctgactgtcactgaccttgagcatatcagccagagtatcggcgatatcctgcgcacaccggtcggctcacgggtgatgcgtcgtgattacggctcgttgctggcgtcaatgattgaccagccgcagaccccggcgcttgagttgcagattaaagtcgcctgttacatggcagtgctgaaatgggaaccccgcgtcaccctgtcatccgtcaccacggcgcgcagttttgacgggcgaatgacggtcacgttaaccggccagcacaacgacaccggccagccactttcattaaccatccctgtgagttgaaaccatgccgattatcgacctgaaccagctacccgcaccggatgtggtcgaggagctggactttgaaagcattctcgctgaacgcaaggcgacactgatttccctttacccggaagatcagcaggaggcggtcgcccgtaccctgacactggaatctgagcctctcgtcaaactgctggaagaaaatgcttatcgtgagcttatctggcgtcagcgtgtgaatgaggccgcacgggcggtgatgctggcctgtgccgccggtaatgaccttgatgtgattggtgccaattacaacaccacgcgcctgactatcaccccggcagatgattcgaccatcccgccgacaccggcagtgatggaatctgacaccgattatcgtctgcgtattcagcaggcttttgagggcttaagcgtcgccgggtcagtgggagcctatcagtatcatggtcgcagtgctgacgggcgtgtcgcggatatttctgtcaccagtccgtctccggcctgtgtcaccatctctgtgctgtcacgtgaaaataacggcgtcgcatccgaagacctgctggctgtggtgcgtaacgcccttaatggcgaggacgtcaggccggtggccgaccgcgtgaccgtgcagtctgccgccatcgttgaataccagataaacgccacgctttacctttaccctggtcccgaaagcgaacccatccgcgctgccgctgtgaaaaagctggaagcgtatatcacggcacagcaccggctggggcgcgacatccgtctgtctgccatttatgccgctttgcatgtggaaggtgtgcagcgtgtcgaactggctgcaccactggccgacatcgtgctcaacagtacgcaggcgtctttctgtaccgaataccgcgtcgtgaccggaggctcggatgagtgattcgcgactgctgccgaccggctcatcaccgcttgaggtcgccgccgcaaaagcctgtgcggaaattgaaaaaacgccggtcagtattcgtgaactgtggaacccggacacctgtccggcaaatctgctgccgtggctggcgtgggcgttttcggtcgacaggtgggatgaaaagtggccggaagcgacaaaacgcgccgttatccgcgatgcctatttcatccactgtcataagggcacgataggtgcaatccggcgtgtggtggagccgctcggctatctcatcaacgtgacggagtggtgggaaaacagtgacccgcccggcaccttccggcttgatattggtgtactggaaagcggtatcacagaggcaatgtatcaggaaatggaacggctgattgctgatgccaaacctgcaagccgtcaccttattggcctgaacattacccgggacattcccggctatctgttcgccggtggtgtggcttacgacggcgatgtaattacggtttaccccggataagtgaggaataatgagcataaaattcagaaccgttatcaccactgccggtgcagcaaagctggcagcggcaaccgcgccgggaaggcggaaggtcggcattaccacgatggccgtcggggatggcggtggtaaattgcctgtcccggatgccggacagaccgggcttatccatgaagtctggcgacatgcgctgaacaaaatcagccaggacaaacgaaacagtaattatattatcgccgagctggttattccgccggaggtgggcggtttctggatgcgtgagcttggcctgtacgatgatgcgggaacgttaattgccgtggcgaacatggccgaaagctataagccagcccttgccgaaggctcaggacgttggcagacctgtcgcatggtcatcatcgtcagcagtgtggcctcagtggagctgaccattgacaccacaacggtgatggcgacgcaggattacgttgatgacaaaattgcagagcacgaacagtcacgacgtcacccggacgcctcgctgacagcaaaaggttttactcagttaagcagtgcgaccaacagcacgtctgaaacactggccgcaacgccgaaagcggtaaaggccgcgtatgacctggctaacgggaaatataccgcacaggacgccaccacagcgcgaaaaggccttgtccagcttagtagcgccaccaacagcacgtctgaaacgctcgccgcaacaccaaaagccgttaagacggtaatggatgaaacgaacaaaaaagcgccattaaacagccctgcactgaccggaacgccaacgacgccaactgcgcgacagggaacgaataatactcagatcgcaaacacggctttcgttatggccgcgattgccgcccttgtagactcgtcgcctgacgcactgaatacgctgaacgagctggcggcggcgctgggcaatgacccgaattttgctaccaccatgactaatgcgcttgcgggtaagcaaccgaaagatgctaccctgacggcgctggcggggcttgctactgcggcagacaggtttccgtattttacggggaatgatgttgccagcctggcgaccctgacaaaagtcgggcgggatattctggctaaatcgaccgttgccgccgttatcgaatatctcggtttacaggaaacggtaaaccgagccgggaacgccgtgcaaaaaaatggcgataccttgtccggtggacttacttttgaaaacgactcaatccttgcctggattcgaaatactgactgggcgaagattggatttaaaaatgatgccgatggtgacactgattcatacatgtggtttgaaacgggggataacggcaatgaatatttcaaatggagaagccgccagagtaccacaacaaaagacctgatgacgttgaaatgggatgcactaaatattcttgttaatgccgtcattaatggctgttttggagttggtacgacgaatgcactaggtggtagctctattgttcttggtgataatgataccggatttaaacagaatggagacggtattcttgatgtttatgctaacagtcagcgtgtattccgttttcagaatggagtggctattgcttttaaaaatattcaggcaggtgatagtaaaaagttctcgctatccagctctaatacatccacgaagaatattacctttaatttatggggtgcttccacccgtccagtggttgcagagttaggcgatgaggccggatggcatttctatagccagcgaaatacagataactcggtaatatttgctgttaacggtcagatgcaacccagcaactggggaaattttgattcccgctatgtgaaagatgttcgcctgggtacgcgagttgttcaattgatggcgcgaggtggtcgttatgaaaaagccggacacacgattaccggattaagaatcattggtgaagtagatggcgatgatgaagccatcttcaggccgatacaaaaatacatcaatggcacatggtataacgttgcgcaggtgtaagttatgcagcatttaaagaacattaagtcaggtaatccaaaaacaaaagagcaatatcagctaacaaagaattttgatgttatctggttatggtccgaagacggaaaaaactggtatgaggaagtgaagaactttcagccagacacaataaagattgtttacgatgaaaataatattattgtcgctatcaccagagatgcttcaacgcttaatcctgaaggttttagcgttgttgaggttcctgatattacctccaaccgacgtgctgacgactcaggtaaatggatgtttaaggatggtgctgtggttaaacggatttatacggcagatgaacagcaacaacaggcagaatcacaaaaggccgcgttactttccgaagcggaaaacgttattcagccactggaacgcgctgtcaggctgaatatggcgacggatgaggaacgtgcacgactggagtcatgggaacgttacagcgttctggtcagccgtgtggatcctgcaaatcctgaatggccggaaatgccgcaataa

SEQ ID NO: 10 - DNA, 6603 bp, Artificial Sequence
  Synthetic Construct
atgagtgactatcatcacggcgtgcaggtgctggagattaacgagggcacccgcgtcatttccaccgtatccacggccattgtcggcatggtctgcacggccagcgatgcagatgcggaaaccttccccctcaataaacctgtgctgattaccaatgtgcagagcgcaatttcaaaggccggtaaaaaaggcacgctggcggcatcgttgcaggccatcgctgaccagtcaaaaccggtcaccgttgtcatgcgcgtggaagacggcaccggtgatgacgaggaaacgaaactcgcgcagaccgtttccaatatcatcggcaccaccgatgaaaacggtcagtacaccggactaaaagccatgctggcggcggagtcggtaaccggtgttaaaccgcgtattctcggcgtgccgggactggataccaaagaggtggctgttgcactggcatcagtctgtcagaagctgcgtgctttcgggtatatcagcgcatggggctgtaaaaccatttccgaggtgaaagcctatcgtcagaatttcagccagcgtgagctgatggtcatctggccggatttcctcgcatgggatacggtcaccagtaccaccgccaccgcgtatgccaccgcccgtgcgctggggctgcgcgctaaaatcgaccaggagcagggctggcataaaacgctgtccaatgtcggggtgaacggtgttaccggcatcagcgcatctgtattctgggatttgcaggagtccggcaccgatgctgacctgcttaacgagtcaggcgtcactacgctgattcgccgcgacggtttccgcttctggggtaaccgtacctgctctgatgacccgctgttcctctttgaaaactacacccgcaccgcgcaggtcgtggccgacacgatggctgaggcgcacatgtgggcggtggacaagcccatcactgcaacgctgattcgcgacatcgttgacggcatcaatgccaaattccgtgagctgaaaacaaacggctatatcgtggatgcgacctgctggttcagcgaagaatccaacgatgcggaaaccctcaaggccggaaaactgtatatcgactacgactatacaccggtgcctcctctcgaaaacctgaccctgcgccagcgtattaccgataaatacctggcaaatctggtcacctcggttaacagcaattaaggagcctgaccgatggcaatgccgcgcaaactcaagttaatgaacgtctttctgaacggctacagctatcagggcgttgcaaagtccgtcacgctgccaaaactgacccgtaagctcgaaaactatcgcggtgcggggatgaacggcagcgcaccggtagacctcggccttgatgacgatgcgctgtcaatggagtggtcgctcggtggcttcccggattcggttatctgggagctttacgccgcaaccggtgtggatgccgtgccgattcgttttgcaggctcttaccagcgcgacgataccggcgaaacggtggccgtcgaagtggtcatgcgtggacgtcagaaagaaatcgacaccggcgagggtaaacagggagaagacactgagtcgaaaatctccgtggtctgcacctatttccggctgacgatggacggtaaggagctggtcgaaattgacaccatcaacatgattgagaaggtgaacggcgtcgatcggctggagcaacaccgccgcaatatcggcctgtgattttcatccggtcagcctggctggccggttaaccctgattcagaagtgagaaaaccatgaacaaagaaaatgtcattaccctggacaatccggtcaaacgtggtgagcaggttatcgaacaggtcacgctgatgaaacccagtgccgggacgctacgcggtgtcagtctggctgcggttgcaaactccgaagtcgatgcactgattaaggtgctgccgcgcatgacggcaccgatgctgaccgagcaggaagtcgccgcgctggaactgcctgaccttgtggcgctggccggtaaggtggtcggttttttgtcgccgaactcggtgcagtgacgtttccgaaaaatctctcggtcgatgacctgatggcggatgtggcagtgatatttcactggccgccatcagaactgtatcccatgagcctgaccgaactcatcacatggcgcgaaaaggcgctccggcgaagcggaaacacgaatgagtaacaatgtaaaattacaggtattgctcagggctgttgaccaggcatcccgcccgtttaaatccatccgcacagcgagcaagtcgctgtcgggggatatccgggaaacacaaaaatcactgcgcgagctgaacggtcacgcatcccgtattgagggattccgcaagaccagtgcacagctcgccgtgactggtcatgcacttgaaaaggcacggcaggaggccgaagcccttgccacacagtttaaaaacaccgaacgtccgacccgtgctcaggcgaaagtcctggaatccgcaaagcgtgcggcggaggacttacaggcgaaatataaccgcctgacagattccgttaaacgccagcagcgggaactggccgctgtgggaattaatacccgcaatcttgcacatgatgagcagggactgaaaaaccgtatcagtgaaaccaccgcacagcttaaccgtcagcgtgatgcgctggtgcgtgtcagtgcgcaacaggcaaaacttaacgcagtaaaacagcgttatcaggccggaaaggaactggccggaaatatggcctcagtgggcgctgccggtgtggggattgcggcggcgggaacgatggccggtgttaagctactgatgcccggttatgagtttgcgcagaaaaactcagaattacaggctgtgatcggagtggcaaaagactccgccgaaatggccgcactccgcaagcaggcgcgccagctcggcgacaataccgccgcctcggcagatgatgcagccggtgcgcagattattattgcgaaagccggtggggatgttgatgccattcaggcggcaacgccggtcacgctgaacatggcgctggcgaaccgtcgcacaatggaagaaaacgccgccctgctgatggggatgaaatccgcctttcagctttcaaacgataaggtcgctcatatcggggatgttctctccatgacgatgaacaaaaccgccgccgattttgacggcatgagcgatgcgctgacctatgccgcacctgtggcaaaaaatgccggtgtcagcattgaagaaaccgccgcaatggtcggggcgctgcatgatgcaaaaatcacaggctcaatggcggggacgggaagccgtgccgtgttaagccgcctgcaggcaccgacgggaaaagcatgggatgcactcaaagagcttggagtgaaaacctcagacagcaaaggaaacacccggccaatatttaccattctgaaagaaatgcaggccagttttgagaaaaaccggctcggtactgcccagcaggctgaatacatgaaaactattttcggggaggaggccagctcagccgctgccgtgctgatgactgccgcctcaaccggaaagctggacaaactgaccgctgcgtttaaagcctcagacgggaagaccgccgagctggtaaatatcatgcaggacaacctaggcggtgactttaaagcgtttcagtccgcttatgaggcggtggggactgacctgtttgaccagcaggaaggcgcgctgcgtaagctcacgcagacggccacaaagtatgtgttaaaactcgacggctggatacagaaaaacaaatcactggcgtcaaccatcggcatcattgccggcggtgcactggcgcttactggcatcatcggtgccattggcctcgtagcctggccggttatcaccggcatcaatgccatcatcgcggcagcaggcgcaatgggggcagtcttcacgacggttggcagtgctgttatgaccgccatcggggctattagctggccggttgtggccgtggtggctgccattgtcgccggtgcgttgcttatccgtaaatactgggagcctgtcagcgcattctttggtggtgtggttgaagggctgaaagcggcatttgcgccggtgggggaactgttcacgccacttaaaccggtttttgactggctgggcgaaaagttacaggccgcgtggcagtggtttaaaaacctgattgccccggtcaaagccacccaggacaccctgaaccgttgccgtgacacgggcgtcatgttcgggcaggcactggctgacgcgttgatgctgccgcttaatgcgttcaacaaactgcgcagtggtattgactgggtactggaaaaactcggtgttatcaacaaagagtcagacacacttgaccagaccgccgccagaactcataccgccacgtatggtaccggtgactatattccggcgaccagctcttatgcaggctatcaggcttatcagccggtcacggcaccggctggccgctcttatgtagaccagagtaaaaacgaatatcacatcagcctgacgggggggactgcgccggggacacagcttgaccgccagttacaggatgcgctcgaaaaatacgagcgggataaacgtgcgcgcgcccgtgccagcatgatgcatgacggttaaggaggtgacgaaaaatgatgctcgcgttaggtatgtttgtttttatgcgccagacgctgccacaccagaccatgcagcgtgaatcagattatcgctggccgtcaaattcccgtatcggtaaacgggatgcctttcagtttctcggtgtgggtgaggaaaacatcacgctggccggtgtgctttatcccgaactgaccggcggcaagctgacgatgaccacgctcaggctgatggcagaggaggggcgggcgtggccgttgctggatggcaccggcatgatttacggcatgtatgtcatcagcagggtgagtgaaacagggagtattttctttgcagacggcacaccccggaaaattgattttacgctgtcactcacccgcgttgatgaatcactggccgcgctttatggcgatatcggtaaacaggcggaatcgctcatcggtaaggccggcagtatggcgaccagattcacaggtatgacgggggcgggataatgctggatgcgctgacatttgatgcaggcagtacgctgacgccggattacatgctgatgctcgacagcagggatattaccggcaatatcagcgaccgtctgatgagcatgaccctgacggataaccggggctttgaggctgaccagcttgatattgaactgaacgatgccgacgggcaggtcgggctgccggttcgtggcgctgtcctgacggtgtatatcggctggaaaggttttgccctggtatgcaaagggaaatttaccgttgatgaggttgaacaccggggcgcaccggatgtagtcaccatccgcgcccggagtgcagattttcgcgggacgctcaattcccgccgggaaggctcctggcatgacaccacgctcggtgcgattgttaaggcgatagccacccgtaacaggctggaagccagtgtcgctccgtcactggccggaataaaaattccacacatcgaccagtcgcaggagtctgatgcgaaattcctgacccgtcttgcagaacgcaacggcggtgaggtgtcggtaaaaatgggaaaactgttgtttctcaaagcggggcagggagtgacggccagcggtaaaaaaatcccgcaggtcaccataacccgcagcgacggcgaccgccatcattttgcgattgctgaccgtggagcctacaccggtgtaacggcaaaatggctacacactaaagacccgaagccgcaaaagcagaaggtaaaactgaaacgcaaaaagaaagagaaacacctgcgcgcactggagcacccgaaagcgaaaccggtcaggcagaagaaagcgcctaaagtaccggaagcgcgtgaaggtgaatacatggccggtgaggctgacaacgtttttgccctgaccacggtatatgccacgaaagcgcaggccatgcgcgccgctcaggcgaagtgggataaactgcaacggggcgttgcggagttctctatcagcctggctaccggtcgggcagatatttacacggaaacaccggtcaaagtgtctggctttaagcgcgtcatagacgagcaggactggacaatcactaaggtgacacattttctgaataatagcggcttcacgacgtccttagagcttgaggtcaggctttctgatgtggagtacgaaacagaagatgatgagtgatgtttttgttttatctgtttgttttgtaaggataaattaactaaaatggcaccatcaacaaaaccggaagaggtgctcgcgatgtttcattgtcctttatgccagcatgccgcacatgcgcgtacaagtcgctatatcactgacacgacaaaagagcgttatcatcagtgccagaacgtgaattgcagcgccacgttcatcacttatgagtcggtacagcgatacatcgtgaagccgggagaagtccacgccgtaaggccgcacccgttgccatcagggcagcaaattatgtggatgtaa

SEQ ID NO: 11 - DNA, 12 bp, Artificial Sequence
  Synthetic Construct
  misc_feature 2, 9: n = A,T,C or G
angatttantcc

SEQ ID NO: 12 - protein, 192 aa, Artificial Sequence
  Synthetic Construct
MKTESYFKEYNQFVLDQHKAIQELEQERNALESKIKLDKSTYKQLIMDGQDDKADNLYQATDADEKKLKALNKRLETKKSVSKEVKYQKTIELLKHQSELSSLYESEKQSAIEKLKKAVDAYNEIIDEIEDINDRYEDEHQQYASVYSQEQLYDDKEARKALNGHFKENIFTSFINGNDLPYEHNNKLFLKC

SEQ ID NO: 13 - protein, 72 aa, Artificial Sequence
  Synthetic Construct
MKTKYELNNTKKVANAFCLNEEDTNLLINAVDLDIKNNMQEISSELQQAEQSKQKQYGTTLQNLAKQNRIIK

SEQ ID NO: 14 - protein, 113 aa, Artificial Sequence
  Synthetic Construct
MDKQQIKDFVCDYHERTRSDVLIDDDINTDEFFSIADENSNEWMADDNIDDHIVKNHLEMIVDRVANDKEFYIFDSLIQGRSYQDISGVLDCSEQSVRFWYETLLDKIVEVIE

SEQ ID NO: 15 - protein, 113 aa, Artificial Sequence
  Synthetic Construct
MESIAEKETYHLPTEHLQVFNVIKNTSNKYITKTKILNQLGYEYNSSNERWLRRVINSLVYDYGYPIGCSYKPSERGYYIITTEQEKQQAMRSIKKLADGSMKRYEALKRIEV

SEQ ID NO: 16 - protein, 191 aa, Artificial Sequence
  Synthetic Construct
MIAYPIRVGSVYRGEQMKLLKTKNCLYYRNGDNKLSEYQLLTQFNPTFINKKIRMCEFQIESMYHMSASTTTCDEMMGVVSVSYPIEKLVIKIIETKARLQNYKNRSISNMVLLKTVLNHYTEKEQKKVVKYMRSNGRYKPYNVIERLQVDLYQASIKQRSERQKQRNIAIENSKIARVNAYHQSSYVKVV

SEQ ID NO: 17 - DNA, 27079 bp, Artificial Sequence
  Synthetic Construct
atgaacgaaaaacaaaagagattcgcagatgaatatataatgaatggatgtaatggtaaaaaagcagcaattacagcaggttatagtaagaaaacagcagagtctttagcaagtcgattgttaagaaatgttaatgtttcggaatatattaaagaacgattagaacagatacaagaagagcgtttaatgagtattacagaagctttagcgttatctgcttctattgctagaggagaacctcaagaggcttacagtaagaaatatgaccatttaaacgatgaagtggaaaaagaggttacttacacaatcacaccaacttttgaagagcgtcagagatctattgaccacatactaaaagtacatggtgcgtatatcgataaaaaagaaattactcagaagaatattgagattaatattggtgagtacgatgacgaaagttaaattaaactttaacaaaccgtctaatgttttcaatagaaacatattcgaaatactaaccaattacgataacttcactgaagtacattacggtggaggttcgagcggtaagtctcacggcgttatacaaaaagttgtactcaaagcattgcaagattggaaatatcctaggcgtatactgtggcttagaaaagtacaatcaacaattaaagatagtttgttcgaagatgttaaagattgtttgataaactttggtatttgggacatgtgcctttggaataagactgataacaaagttgaattgccaaacggcgcagtttttttgtttaaaggattagataacccagagaaaataaagtcgataaaaggcatatcagacatagtcatggaagaagcgtctgaattcacactaaatgattacacgcaattaacgttgcgtttgagggagcgtaaacacgtgaataagcaaatatttttgatgtttaacccagtatctaaactgaattgggtttataagtatttctttgaacatggtgaaccaatggaaaatgtcatgattagacaatctagttatcgagataataagtttcttgatgaaatgacacgacaaaacttagagttgttagcaaatcgtaatccagcatattacaaaatttatgcgttaggtgaatttgctacactagacaaattggttttccctaagtatgaaaaacgtttaataaataaagatgagttaagacatttaccttcttattttggattggactttggctacgttaatgatcctagtgcttttatacattctaaaatagatgtaaagaaaaagaagttatacatcattgaagagtatgttaaacaaggtatgctgaatgatgaaatagctaatgtcataaagcaacttggttatgctaaagaagaaattacagcagatagtgcagaacaaaaaagtatagctgaattaaggaatctagggcttaaaaggattttaccaaccaaaaaagggaagggctcggttgtacaagggttacaattcttaatgcaatttgaaatcattgttgatgaacgttgtttcaagactattgaagagtttgacaactacacatggcaaaaggacaaagatacaggtgaatataccaatgaaccagtagatacatacaatcattgtatcgattcgttgcgttattcagtggaacgattctacagaccggttagaaaacgcacaaatctcagttcgaaagttgacacaataaaatctctaggattataggagggaacaaatgttaaaagtaaacgaatttgaaacagatacagatctacggggaaacataaattacttatttaatgatgaagccaatgttgtttacacatatgacgggacggaatccgatttattacaaaacgttaatgaagtaagtaaatacattgaacatcacatggattaccaacgacctagattgaaagtgttaagtgattattacgaaggtaaaactaagaacttagttgagttaacacgacgcaaagaagagtacatggcagataaccgtgtagcgcatgattacgcatcttatattagcgattttatcaacggctatttcttgggtaatccaattcaatatcaagatgatgacaaagatgtattagaagttattgaggcgttcaatgatttaaatgatgttgagtcacacaatagatctttaggattagatttgtcaatttatggcaaagcttatgagttaatgattagaaaccaagatgatgaaacgcgtttatacaagagtgatgcaatgagtacttttgtcatatacgacaatacaattgaacgtaatagtatcgcaggcgttagatatttaagaactaaaccaatagacaagactgacgaagatgaagtgtttacagttgatttattcacttcacacggtgtttatagatatcttaccagtagaacaaatggattgaagctcacaccacgtgaaaacggttttgaatcacactctttcgaacgtatgcctattacagaatttagcaacaacgaaagaagaaaaggggattatgagaaagtaatcactttaattgatttgtatgataatgctgaatcagatactgctaactatatgagtgatttaaatgacgctatgttacttattaaaggtaatttaaatttagatcctgtagaagttagaaaacaaaaggaagctaacgtgttgtttttagaaccgactgtttatgctgatagcgaaggtagagaaacagaaggctctgttgatggtggttatatttataagcaatacgatgtacaaggtaccgaagcttataaagaccgtttaaacagtgatatacacatgtttaccaacacgcctaacatgaaagatgataactttagcggcactcaatcgggcgaggcaatgaaatacaaattatttggattggaacaacgtactaaaactaaagaaggattgtttactaaagggttaagacgtcgtgctaagttgttagagacaatacttaaaaatacatggtcgattgacgctaacaaagatttcaatactgttagatacgtatacaacagaaacttacctaaatcattgattgaagaattaaaagcttatattgattctggtgggaagattagccaaacaactttaatgtctctattctcgttcttccaagaccctgaattagaagttaagaaaatcgaagaagatgagaaagaatctattaaaaaagctcaaaaaggtatttataaagaccctagagacatcaatgatgacgaacaagatgatgatacaaaagatactgttgataaaaaggaatgattgtaattgcctaacaaaaacactcaagaatattgggaagaacgcggacgcaaagcaatcgagaatgagttgaagcgtgataaaactaaagctgaagaaatagaacgtatattgaatatgatgattaagcgcattgaaaaagagatcaatgcgtttattgtcaagtacggagattttgcaggcgttacattacaagaagcacaaaagattattgatgagttcgatgtaaaagcgtttcaagaagaagcaaaaagattggtcgaaaacaaggagtttagcgatagagcaaatgaagaattaaagaagtataacacgaaaatgtatgtatctagagaacagatgttaaagattcaaatagaattcttaattgcttatgcaacagctcaaacagaattatcgatgagggaatatttcgaatcaacagcttatcgtgtgttcagtgatcaagcgggtattttaggtgaaggtgtacaagtagctaaagaagttatagatacaatcgttgatacacaatttcatggtgtcgtttggtcagagcgattatggactaataccgaagcaatgaaacaagaagtagaagaaataattgctaatgtagttattagaggtcgacatcctaatgaatatgttaaagatatgcgcaagcacttaaataaattcgaaggcacagcacgacaaaagaccgcagcaattaaatcattgctttatacggaatcggcacgtgttcacgcacaatcaagcattgacagcatgaaagaaatttcaccggaaggatattatatgtatattgcaaaaatcgataatagaacaactaaagtatgcaaagggcttaatggagaaatattcaaagttaaagacgctaaaattggtgttaatttctatcctatgcatatcaattgtcgttcagattgcgctttactacctaaatctatgtggccgaaaaaaccaagcaagaaacgaaaaacaaaatacttcggagggaaagtgaaaagcggtgattgatttaaaagtgaagttttttaaaggcaagttagttttgtatgacagtaaattaaatgtttggaggatactaatatgagtaatactgacaaataccttagagacatagcaagagaattaaaaggtatacgtaaagagttacaaaagcgaaacgaaacagttattattgatgcaaacttagacagtttaaggtcggcagtattagccgataaagaaaaatcgaaatataatgaacctctcttttaatagctagcacttaattgtgttggctattttttatgtccaaaacgtgctgatgacataaaaagcacgcatggaaaaacagtcgacagactataaatggaggtatatctcatggaagaaaataaacttaagtttaatttgcaattttttgcagaccaatcagatgatccggacgaaccaggcggagatggtaaaaaaggaaatcctgataagaaagaaaatgacgaaggtactgaaataactttcacgccagagcaacaaaagaaagttgatgaaatacttgaacgtcgtgtagcccacgaaaagaaaaaagctgatgagtatgcaaaagaaaaagcagcagaagctgctaaagaagctgctaaattagcgaaaatgaacaaggatcaaaaagatgaatatgaacgcgaacaaatggaaaaagaactggaacaattacgttcagaaaaacaattaaacgaaatgcgttcagaagcacgaaaaatgttgagtgaagcggaagttgattcatcagatgaggttgtcaatttagttgtaacagatactgctgaacaaactaaattgaatgttgaagctttttctaatgcagtaaaaaaagcggttaatgaagcggttaaggttaacgctagacaatcgccattgactggtggagattcatttaatcactcgactaaaaataaaccgcaaaacttagctgaaatagctagacaaaaaagaattattaaaaattaacggaggcatttaaatggaacaaacacaaaaattaaaattaaatttgcaacattttgcaagtaacaatgttaaaccacaagtatttaaccctgacaatgtaatgatgcatgaaaagaaagatggcacgttgttaaacgactttacaacacctatcttacaagaggttatggaaaactctaaaatcatgcaattaggtaagtacgaaccaatggaaggtactgagaagaagtttactttttgggctgataaaccaggtgcttactgggtaggtgaaggtcaaaaaatcgaaacgtctaaggctacttgggttaatgctacaatgagagcgtttaaattaggggttatcttaccagtaacaaaagaattcttgaattacacttattcacaattctttgaagaaatgaaacctatgattgctgaagctttctataaaaagtttgacgaggcaggtattttgaatcaaggtaacaatccgttcggtaaatcaattgcacaatcaattgaaaaaactaataaggttattaaaggtgacttcacacaagataacattattgatttagaggcattgcttgaagatgacgaattagaagcaaatgcatttatctcaaaaacacaaaacagaagcttgttacgtaaaattgtagatcctgaaacgaaagaacgtatttatgaccgtaacagtgattcgttagacggtctacctgtggttaaccttaaatcaagcaacttaaaacgtggtgaattaatcactggtgacttcgacaaattgatttatggtatccctcaattaatcgaatacaaaatcgatgaaactgcacaattatctacagttaaaaacgaagatggcacacctgtaaacttgtttgaacaagacatggtggcattacgtgcaactatgcatgtagcattgcatattgctgatgataaagcgtttgctaagttagttcctgctgacaaaagaacagattcagttccaggagaagtttaataaataattaggagtggtaacatgcccgaaatcattggaattgttaaagtagattttacagatttagaagataacagacatgtctatatgaaagggcatgtctaccctcgtaaaggttataatcctacagatgaacgtatcaaagctttagctagtgttgaaaataaacgcaacaaacaaatgatttacattgtaaatgacaaattaaccaaaaaagaacttgtcgaaatagcaagtgttgctggcttacaagttgatgaaaaacaaacaaaagctgaaattatcaatgcttttgagtcactagagtaggtggttatatgactacgctagctgatgtaaaaaaacgtattggtcttaaagatgaaaagcaagatgaacaattagaagaaatcataaaaagttgtgaaagccagttgttatcaatgttacctattgaagttgaacaaataccggaaaggtttagttacatgattaaagaagttgcagttaaacgctacaacaggattggtgctgaaggtatgacatcagaagcggttgacggacgtagcaatgcgtatgaattgaacgatttcaaggagtatgaagctattattgataattactttaatgctagaacgagaactaaaaaaggaagggctgtgttcttttgagatatgaagatagagttatttttcaattagaacaagtagcaacttacaatcctaaaactagcaaaaaagaaaacacactaatcacttatgatgcgataccatgcaatattaaccccatttctagagcaagaaagcaacttgaatttggtgatgtaaaaaacgatgtaagtgttctgaggataaaagaatcaatatcttaccctgttagccacgtgttggttaatggcattcgctacaagatagttgatacaaggatatacagacacgaaacgtcatattatatcgaagaggtcaattgatgaatatagatggattagacgcactgttaaaccaatttcacgatatgaaaaccaacattgatgatgatgtagatgatattttacaggaaaacgccaaagaatatgtagtacgagctaaattgaaagctagagaagtaatgaataagggttattggactggtaatttatcacgcaatatcagatataaaaaaactggcgatttgcaatacactatcacatcgcacgcagcttatagtggtttcttagaatttggtactcgatacatggaggctgaaccttttatgtggccggtatacgaagtgattaggaaatcaactgtagaagaattgaaagcgttgtttgaataggagataaaagcatgacaccgaacttacaactttataataaagcgtatgaaacgctacaaggatatggattccctgttatttctcgtaaagagatgcaacaagagattccgtatcctttttttgtaataaaaatgccggagtcaaatagaagtaagtacacgtttgatagttattctggcgatacgaatttagttattgatatttggagtgtaagcgatgatttaggacatcatgacggacttgttaaaagatgtattgatgatttaacacctagcgttaaaacaaacgattatgactttgaagaagaagatactaacatcacacagttagttgatgatactaccaatcaagaattgctacacacatcagtaacgatatcttacaaaacattttaaaaaacggaggaatattgaatggcaaatatgaaaaatagtaatgatcgtattattttatttagaaaagctggcgaaaaagtagatgctactaaaatgctttttttaactgaatacggcttatcacatgaagctgatacagatacagaggatacaatggacggttcttataacactggtggttctgttgagtcaacaatgtctggtactgctaaaatgttttatggtgacgattttgcagatgaaattgaagatgcagttgtagatcgcgtattgtatgaagcttgggaagttgaaagtagaataccaggcaaaaatggggattccgctaaatttaaagcgaaatatttccaaggtttccacaataaatttgaattaaaagcagaagctaacggtattgatgaatatgaatatgaatatggagtgaatggtcgtttccaacgtggatttgcaacactacctgaggctgtaacaaagaaacttaaggcgactggatacagattccacgacactacaaaagcagatgcattaactggcgaagatttaacagcaattccacaacctaaagtagattcaccaccggttgcaccaagagaggtataaaaatagggcgttaagccctttttattttgtttaaattaattatgaatggagattttaagttatgaatgtagaaattaacggaaagtcattagaattaagttttggttttaaatttttaagagaaatcgataaccgattaggtttaaaagttgaacaagcttctatcggtcaaggtgtatcaatgttgcctgtaggtttagaaagtggaaatccggttgtgattggcgaagttttaatcgcagctacatctcacttaaaaaaacaagcaattactattaataacattgatgaagcattagatgaaatcgcagaaaatatcggactagaagaattcggttcggatattttaacggagttgggaaagcgacctatgacccgaaacctagtcgaagtagtggaaacggaagaaaaaccagcggaagcctaataacttacgacagaatcgttataacttgtatgtcaacacttggtattacagatttgaacgttattgagcaaatgacattaacagaatataactatcgaatgtatgcgaaagagtatgaaatgctaacccaagaattcgaacgttacaaacttgcgtttgctattcgtgatgctgcagctactaaaaatgttgggacagaaaataaacctaaagaggaatatgtttttaacaatgcaaacgacgtattgccttatgaagaaaatatccaacggcttaacgaaggtaaagatataagatttagtagcgaacgtgatgaatacgaaccacaaaataatgaattctttaaagttatagcagaatttaataagcaatagaaagagaggtgttaatgtgacggaatataaaattaaagcgactattgaagctagtgtagccaaattcaaaaggcaaattgatagtgcggttaagtctgtgcaaagatttaaacgagtagcagatcaaactaaagatgttgaattaaacgctaacgataaaaatttacaaaaaactatcaaagttgctaaaaagtctttagatgcctttagtaacaaaaatgtaaaagctaaattagatgctagtatacaagatttacaacaaaaggtactagaatcgaattttgaactagacaaactaaactctaaagaagttacaccagaagttaagttgcaaaaacaaaagttgattaaagatatcgctgaaacagaagctaaattatcagaattagaaaagaaacgtgtcaatattgacgtcaatgcagataacagtaaattcaatcgagtgttaaaagtatctaaagctagtctcgaagcattaaataggtctaaagccaaagctattatagacgtggacaatggtgttgctaactctaaaatcaaacgtactaaagaagaacttaaaagtattccgaacaaaactagatctcgacttgatgtagatacagggctttctataccaactatttatgcgtttaaaaaatcattagacgcattgccgaacaaaaaaacaacaaaggtagatgtcgatactaatggtttaaagaaagcttatgcctacataataaaagcaaatgacaattttcaaagacagatggggaatttagctaatatgttccgtgtgttcggtactgtaggttctaatatggttggtggattacttacatcatcttttagtatcttaatacctgtaatagcgagcgtagtacctgtagtatttgcgctattaaacgctatcaaagtgttaactggtggtgtacttgctttaggtggtgccgtagcaatagcgggagcaggatttgtagcgtttggcgcaatggctatcagcgctataaagatgcttaatgatggcactttacaagctagctcagcaacaaacgaatacaaaaaagcgttagatggcgtaaagtcagcatggactgatattataaagcaaaatcaatccgctatcttcacaactcttgcaaatggtttaaatactgttaaaactgcaatgcagagcttacaaccgttttttagtggtatttcaagaggaatggaagaagcgtctcaaagcgtgcttaaatgggctgaaaatagcagtgtagcttcaagattctttaatatgatgaatacaacgggtgtttcggtatttaacaagctattaagtgctgcaggtggttttggtgacggattagtcaatgtattcacgcaattagcaccactgtttcaatggtcggctgattggttggatagattaggtcaatctttctctaactgggctaatagtgcagctggagaaaattcgataactcgttttattgaatacacaaaaacaaacttacctatcattggtaatattttcaaaaatgttttcgttggaattaacaatttgatgaatgcattcagcggatcatcaactggcatattccaatctcttgaacaaatgacagctaagtttagggaatggtctgaacaagtaggacaatctcaagggtttaaagactttgtcagttatatacaaacaaatggaccactaataatgcaattgattggaaacatcgcaagaggattagttgcattcgcaacagcaatggctcctatagctagtgcagtattacgcgttgcagttgcaataactggttggatagctaacttgtttgaggcgcatccagctacagcacaattagttggtgtcattataactttagttggtgcatttagatttttaataccgattattcttgctgtatctaactttatgggtggcggattaataggtagaatcattgcattagtaagtaagttcggtttattaagagcgggattaacaattttaaaaggtgcgttcatgttattaaaaggaccattaaaaattatatcagttatattccaattgttattcggtaagattggattaattagaaatgctatcacaggactagtaactgtgtttggtattttaggcggtccaataacaatagtaattggtgtaattgctgcattaatagctatattcgttttattgtggaataaaaatgaaggattcagaaactttattataaatgcttggaatgcgataaaaacgtttatggttaatgtttggaatgtattaaaagctgtagcttcggttgtatggaatgctattttaacagctatcactacagcagtatcgaatgtttacaattttataatgattgtttggaatcaaatagtcgcttatttacaagggctatggaatggaattatcgctattgcaacaacagtatggaaccttttagttacaatcattacaactgttttcacgacgataatgacaatagttatgacgatatggacagctatttggacgttcttaagtacaatctggaatacgataattacaatcgctactacgatttggaatttgttagtcactgtaataactacagtgtttaccacaattatgactatcgcaataacaatttggaacgctatttggacgttcttacaaacgttgtggaacactatagttactgtggcaactaaggtttggaacgctatcactacagctatatctactgcgttacaagcggcatggagttttatttctaatatatggaatacgatttggagtttcttatctggtatattaacgacaatttggaataaagttgtaagcatattcacacaagttgtttcaactatatcagacaaaatgtctcaagcttggaacttcattgtcactaaaggtatgcaatgggtatctactataacaagtacgctaattaactttgttaatagagttgttcaaggattcgttaatgttgtaaacaaagttagtcaaggtatgacaaatgcagtaaataaagttaaaagctttgtggatgactttgtatcagcaggtgctgatatgatccgtggtttgatgagaggtattggtaatatggctagagacttagctgaaaaagcagctagtgtagcaaaaggtgctttaaatgcagccaaaagagcgctaggtattcactcaccttcacgtgaattcatggatgttggtatgtattcaatgttaggtttcgttaaaggtatagataatcattcaagtaaagttatccgtaatgtttctaatgttgcagataaagtagttgatgcatttcaacctacattaaacgcacctgacatttctagtattacaggaaacttaagtaatttaggtggaaatataaatgcgcaagtacaacacacacattctattgaaacatcaccgaacatgaaaactgttaaagttgaattcgatgtcaataacgatgcgcttactagtattgttaacggcagaaatgctaaacgcaattctgagtattacttataaaggaggttacaaatggacatagaattaacaaaaaaagatggtactgtaatcaaattaagtgaatacgggtttatcgttaacgatatagtaattgatagcatgcaaatcaacacaaagtatcaagacaaagaaaatatgaacggtcgtatattaatggggagcaattatatcagtagagatatagttgttccttgtttttgtaaagttaaaaatcgttcagacattgcttatatgcgagatatgttgtattcgttaacgacagacatagaacctatgtatttgcgagaaatcagaagaaaagaagagttgaattacaggtttactcaaccaacttctgatgattacgtgaaattagataaaaacaacttcccggattacgaatattcaagacacgatcaacaaatttatgtaaatggtaaacagtataaagttatttttaacggagttataaaccctaaacaaaaaggtaataaagtttcttttgaactaaaattcgaaactacagaattaccatacggtgaaagtattggaacaagcctagagttagaagaaaacaaaaaggttggattgtggtcgtttgattttaatattgattggcatgcaggcggagacaaaagaaagtatacatttgaaaatttgagcaaaggtacagtttactatcatggtagtgctcctaacgaccaattcaacatgtataaaaagataacaattattttaggcgaagatacagaatcgtttgtatggaatttaacgcatgctgaaataatgaaaatcgaagggatcaaactaaaagctggagacagaattgtttatgatagcttccgagtttataaaaacggtgttgaaataagtaccgaaacgaatatagcccaaccaaaatttaaatacggagctaataaatttgagtttaatcaaacggtacaaaaagttcagtttgatttgaaattttattataagtaggtgtcagaatgacaataactattaaaccacctaaaggtaatggcgcacctgtaccagtagaaacaactttagtaaaaaaagttaatgctgacggtgtattaacttttgatattctagaaaataaatatacttatgaagttattaacgctatagggaaaagatggattgttagtcatgtcgaaggtgaaaacgacaagaaagaatatgtaataactgtcattgataggaaatcagaaggcgacagacaactggttgaatgtactgctagagagattcccatagacaagttaatgattgatagaatttatgttaatgtaacaggatcttttacagtagaaagatattttaacattgtgtttcaaggtactggaatgctttttgaagtcgagggcaaagttaaatcttcaaagtttgaaaatggtggtgaaggcgatacaaggttagaaatgtttaaaaagggattagaacatttcggtttagaatataaaataacgtatgacaaaaagaaagacagatataagtttgtattgacgccttttgcaaatcaaaaagcgtcttattttatttctgacgaagtcaacgccaacgctataaaactcgaggaagatgcaagtgatttcgccaccttcattagaggatatggtaattattcaggagaagaaacattcgaacacgctgggctcgtaatggaagctagaagtgcattagctgaaatatacggcgacatccacgcagaaccatttaaagatggtaaagtgactgaccaagaaactatggataaagaattacaatcgagattgaaaaagtcgttaaaacaatctttgtctttggactttttggtgttaagagaatcatatccagaagcagacccacaacccggagacatagtacaaataaaatctaccaaactaggtttgaatgatttagtccgtatagtacaagttaaaacgattaggggtataaacaatgtaattgttaagcaagatgtaacgcttggtgagtttaatcgagaacaacgatatatgaaaaaagttaatactgcagctaactatgtttctggattaaatgatgttaacctttctaatcctagtaaagcggcagaaaacttgaagtctaaagtagcgtcaatagctaaatcaacactcgatttgatgagtagaactgatttgattgaagataaacaacagaaggtaagctctaaaactgtgactacatctgacggcactatcgttcatgattttatagataaatcaaacattaaagatgtaaaaacgattggaacgattggcgattctgtagctagaggatcacatgcgaaaactaatttcacagaaatgttaggcaagaagttaaaagctaaaacgaccaaccttgcaagaggtggcgcaacaatggcaacagttccaataggtaaagaagcggtagaaaacagcatttatagacaagcagagcaaataagaggagacctaatcatattacaaggtacagatgatgactggttacatggttattgggcaggcgtaccgataggcactgataaaaccgacactaaaacgttttacggcgccttttgttctgcaattgaagttatcaggaaaaataatccagcttcaaaaatacttgtaatgacagctactaggcaatgccctatgagtggtacaacgatacgccgtaaagatacggacaaaaacaaactagggttaactttagaggattatgtcaatgctcagatattggcttgtagtgaattggatgtaccagtatatgatgcttatcacacagattatttcaaaccatataatccagcatttagaaaatctagcatgcctgatggattacatcctaatgaaagaggtcatgaagttattatgtatgagcttattaaaaattattatcagttttatggatagtaaaggaggaaaacatgagtaataaactaattacagatttaagtagagtctttgactacagatatgtagatgaaaatgagtataactttaaacttatttcagacatgctgacggattttaatttctctcttgaataccacagaaataaagaggtattcgcacatgatggagaacaaataaagtatgaacatttaaatgttacaagtaacgtctctgactttttaacatatttaaacggtcgatttagcaacatggtactaggtcataacggcgacggtatcaacgaagtaaaagacgcgcgcgttgataatacaggttatggtcataagacattgcaagatcgtttgtatcatgattattcaacactagatgttttcactaaaaaggttgagaaagctgtagatgaacactataaagaatatcgagcgacagaataccgattcgaaccaaaagagcaagaaccggaatttatcactgatttatcgccatatacaaatgcagtaatgcaatcattttgggtagaccctagaacgaaaattatttatatgacgcaagctcgtccaggtaatcattacatgttatctagattgaagcccaacggacaatttattgatagattgcttgttaaaaacggcggtcacggtacacacaatgcgtatagatacattgatggagaattatggatttattcagctgtattggacagtaacaaaaacaacaagtttgtacgtttccaatatagaactggagaaataacttatggtaatgaaatgcaagatgtcatgccgaatatatttaacgacagatatacgtcagcgatttataatccggtagaaaatttaatgatttttagacgtgaatataaacccactgaaagacaacttaagaattcgttgaactttgttgaggttagaagtgctgacgatattgataaaggtatagacaaagtattgtatcaaatggatatacctatggaatacacttcagatacacaacctatgcaaggtatcacttatgatgcaggtatcttatattggtatacaggtgattcgaatacagccaaccctaactacttacaaggcttcgatatcaaaacgaaagaattgttatttaaacgtcgcatcgatataggcggtgtgaataacaactttaaaggagatttccaagaggctgagggtctagatatgtattacgatctagaaacaggacgtaaagcacttctaatcggggtaactattggacctggtaacaacagacatcattcaatttattctatcggtcaaagaggtgtaaaccaattcttgaaaaacatcgcacctcaagtatcaatgactgattcaggcggacgtgttaaaccgttaccaatacagaacccagcatatctaagtgatattacggaagttggtcattactatatctatacgcaagacacacaaaatgcattagatttcccgttaccgaaagcgtttagagatgcagggtggttcttggatgtactgcctggacactataatggtgctctaagacaagtacttaccagaaacagcacaggtagaaatatgcttaaattcgaacgtgtcattgacattttcaataagaaaaacaacggagcatggaatttctgcccgcaaaacgccggttattgggaacatatccctaagagtattacaaaattatcagatttaaaaatcgttggtttagatttctatatcactactgaagaatcaaaccgatttactgattttcctaaagactttaaaggtattgcaggttggatattagaagtaaaatcgaatacaccaggtaatacaacacaagtattaagacgtaataacttcccgtctgcacatcaatttttagttagaaactttggtactggtggcgttggtaaatggagtttattcgaaggaaaggtggttgaataatggtagtagataatttttcgaaagatgataacttaatcgagttacaaacaacatcacaatataatccggttattgacacaaacatcagtttctatgaatcagatagaggaactggtgttttaaattttgcagtaactaagaataacagacccttatctataagttctgaacatgttaaaacatctatcgtgttaaaaaccgatgattataacgtagatagaggcgcttatatttcagacgaattaacgatagtagacgcaattaatgggcgtttgcagtatgtgataccgaatgaatttttaaaacattcaggcaaggtgcatgctcaggcattctttacacaaaacgggagtaataatgttgttgttgaacgtcaatttagcttcaatattgaaaatgatttagttagtgggtttgatggtataacaaagcttgtttatatcaaatctattcaagatactatcgaagctgtcggtaaagattttaaccaattaaagcaaaatatggctgatacacaaacgttaatagcaaaagtgaatgatagtgcgacaaaaggcattcaacaaatcgaaatcaagcaaaacgaagctatacaagctattactgcgacgcaaactagtgcaacacaagctgttacagctgaattcgataaaatagttgaaaaagagcaagcgatttttgaacgtgttaacgaagttgaacaacaaatcaatggcgctgaccttgttaaaggtaattcaacaacgaattggcaaaagtctaaacttacagatgattacggtaaagcaattgaatcgtatgagcagtccatagatagcgttttaagcgcagttaacacatctaggattattcatattactaatgcaacagatgcgccagaaaagacggatataggcacgttagagaagcctggacaagatggtgttgatgacggttcttcgttcgatgaatcaacttatacatcaagcaaatctggtgtgttagttgtttatgttgttgataataatactgctcgtgcaacatggtacccagacgattcaaacgatgagtacacaaaatacaaaatctacggcacatggtacccgttttataaaaagaatgatggaaacttaactaagcaatttgttgaagaaacgtctaacaacgctttaaatcaagctaagcagtatgtagatgataaattcggaacaacgagctggcaacaacataagatgacagaggcgaatggtcaatcaattcaagttaacttaaataatgcgcaaggcgatttgggatatttaactgctggtaattactatgcaacaagagtgccggatttaccaggtagcgttgaaagttatgagggttatttatcggtattcgttaaagatgatacaaacaagctatttaacttcacaccttataactctaaaaagatttacacacgatcaatcacaaacggcagacttgagcaacagtggacagttcctaatgaacataaatcaacggtattgttcgacggtggcgcaaatggtgtaggtacaacaatcaatctaactgaaccgtacacaaactattctattttgttggtaagtggaacttatccaggtggcgttattgagggattcggactaaccgcattacctaacgcgattcaattgagtaaagcgaatgtagttgactcagacggcaacggtggcggtatttatgagtgcttactatccaaaacaagtagcactactttaagaatagataacgatgtgtactttgatttaggtaaaacatcaggttctggagcgaatgccaacaaagttactataactaaaattatggggtggaaataatgaaaatcacagtaaacgataaaaacgaagttatcggattcgttaatactggcggtttacgcaatagtttagatgtagatgataacaatgtgcctattaaatttaaagaagagttcgaacctagaaagtttgttttcactaacggcgaaattaaatacaatagcaatttcgaaaaagaagacgtaccgaatgcatcaaaccaacaaagtgcgtcagatttaagtgatgaggaacttcgcggaatggttgcgagtatgcaaatgcaggtggcacaagtaaacgtattaacaatggaattagctcaacaaaacgctatgttaacacaacagttgactgaactgaaaactaacaaaacaagtactgagggggacgtttaaataatgaagatgatttatccaacttttaaagacattaaaactttttatgtttggggttactataaaaacgagcaaattaagtggtacgtagacaagggtttaatcgataaagaagaatacgctttaatcactggagaaaaatatccagaaacaaaagatgaaaagtcacaggtgtaatgcttgtggctttttaatttgaataaagtgggtggcataatgtttggatttaccaaacgacatgaacaagattggcgtttaacgcgattagaagaaaatgataagactatgtttgaaaaattcgacagaatagaagatagtcttagagcgcaagaaaagatttatgacaaattagatagaaattttgaagaattaaagcgcgacaaggtagaagatgaaaagaataaagaaaagaatgccaagaatattagagacataaaaatgtggattctaggtttgatagggactatcttcagtacgattgtcatagctttactaagaactgtttttggtatttaaaggaggtgattaccatgcttaaagggattttaggatatagcttctgggcgtgcttctggtttggtaaatgtaaataacagttaagagtcagtgcttcggcactggctttttattttgattgaaatgaggtgcatacatgggattacctaatccgaaaaatagaaagcccacagctagtgaagtggttgaatgggcgttatatatcgctaaaaacaaaatagctattgatgtacctggttctggaatgggagcacaatgctgggatttacctaattatttactcgataaatattgggggtttagaacatggggaaatgctgatgctatggctcaaaaatccaattatagaggtagagatttcaagataattagaaatacaaaagattttgtaccacaaccaggcgactggggtgtttggactggtggttgggcaggacatgtaaacattgtagtgggaccatgcacaaaagactattggtatggcgtagatcaaaactggtatacaaataacgcaacaggaagtccaccttataaaattaaacactcttatcatgatggaccaggtggaggggttaaatattttgttagaccaccatatcatccagacaaaactacaccggcacctaaaccagaagatgatagtgatgataacgaaaaaaataataaaaaagttccaatttggaaagatgtaacaactataaagtacactatttctagccaagaggttaattatccagaatatatttatcactttatagtagaaggtaatcgacgactcgaaaaacctaaaggaataatgattagaaacgcacaaacgatgagctcggtagaaagtttatataacagtaggaagaaatacaaacaggatgtagaatatccccacttttatgttgatagacataatatttgggcacctagaagagctgtatttgaagttcctaatgaacctgattatatagttatagacgtatgtgaagattatagtgcgagtaaaaatgaatttatttttaatgagattcacgcaatggttgtagctgtagatatgatggccaaatatgagatacctctaagtattgaaaatttaaaagtagacgacagcatttggcgttcaatgttggaacatgttaattggaatatgattgacaacggtgttcctcctaaagataaatacgaagcattagaaaaggcattacttaatatatttaaaaacagagaaaaattattaaattctataactaagccaacagtaacaaaatctagaataaaagttatggtagataataaaaacgctgatatagctaatgtaagagactcgtcaccaacagccaacaatggttcggcatctaaacaaccgcagatcataacagaaacgagtccttatacattcaaacaagcactggataaacaaatggcaagaggtaacccgaaaaaatctaatgcttggggttgggctaacgctacacgagcacaaacgagttcagcaatgaatgtaaagcgtatatgggaaagtaacacacaatgctaccaaatgcttaatttaggcaagtatcaaggtgtttcagttagcgcacttaataagatacttaaaggtaagggaacattgaataatcaaggtaaagcgttcgcagaagcttgtaaaaagcacaacattaatgaaatttatttaatcgcgcatgctttcttagaaagtggatatggaacaagtaacttcgctaacggaaaagatggagtatacaactacttcggcattggcgcttacgacaacaatcctaactacgcaatgacgtttgcaaggaataaaggttggacatctccagcaaaagcaatcatgggcggtgctagcttcgtaagaaaggattacatcaataaaggtcaaaacacattgtaccgaattagatggaatcctaagaatccagctacccaccaatacgctactgctatagagtggtgccaacatcaagcaagtacaatcgctaagttatataaacaaatcggcttaaaaggtatctacttcacaagggataaatataaataaagaggtgtgtaaatgtacaaaataaaagatgttgaaacgagaataaaaaatgatggtgttgacttaggtgacattggctgtcgattttacactgaagatgaaaatacagcatctataagaataggtatcaatgacaaacaaggtcgtatcgatctaaaagcacatggcttaacacctagattacatttgtttatggaagatggctctatattcaaaaatgagccccttattatcgacgatgttgtaaaaggtttccttacctacaagatacctaaaaaggttatcaaacacgctggttatgttcgctgtaagctgtttttagagaaagaagaagaaaaaatacatgtcgcaaacttttctttcaatatcgttgatagtggtattgaatctgctgtagcaaaagaaatcgatgttaaattggtagatgatgctattacgagaattttaaaagataacgcgacagatttattgagcaaagactttaaagagaaaatagataaagatgtcatttcttacatcgaaaagaatgaaagtagatttaaaggtgcgaaaggtgataaaggtgaaccgggacaacctggagcaaaaggtgaagcaggtaaaaaaggagaacaaggcgcacccggtaaaaacggtactgtagtatcaatcaatcctgacactaaaatgtggcaaattgatggtaaagatacagatatcaaagcagaacctgagttattggacaaaatcaatatcgcaaatgttgaagggttagaaaataaattgcaagaagttgaaaaaatcaaagatacaactctcaacgactctaaaacgtatacggatacaaaaattgctgaactagttgatagcgcgcctgaatctatgaacacattaagagaattagcagaagcaatacaaaacaactctatttcagaaagtgtattgcaacagattggctcaaaagttaatacagaagattttgaggaattcaaacaaacactaaatgatttatatgctccaaaaaatcataatcatgacgagcggtatgttttgtcatctcaagcttttactaaacaacaagcggataatttatatcaactaaaaagcgcatctcaaccgacggttaaaatttggacaggaacagaaaatgaatataactatatatatcaaaaagacccgaatacgttatatttaattaaagggtgatttttatggaaggtaattttaaaaatgtaaagaagtttatttacgaaggtgaagaatatacaaaagtatatgctggaaatatccaagtatggaaaaagccttcatcttttgtaataaaacccttacctaaaaataaatatccggatagcatagaagaatcaacagcaaaatggacaataaatggagttgaacccaataaaagttatcaggtgacaatagaaaatgtacgtagcggtataatgaggatttcgcaaactaatttagggtcaagtgatttaggaatatcaggagtcaatagcggagttgcaagtaaaaatatcaactttagtaatccttcagggatgttgtacgtcactataagtgatgtttattcaggatctccgacattgaccattgaataattttaaacgactaatttttagtcgtttttttattttggataaaaggagcaaacaaatggatattaactggaaattgagattcaaaaacaaagcagtactaactggtttagttggagcattgttgctatttatcaagcaagtcacggatttattcggattagatttatctactcaattaaatcaagctagcgcaattataggcgctatcctcacgttacttacaggtattggcgttattactgacccaacgtcaaaaggcgtctcagattcatctatagcacagacatatcaagcgcctagagatagcaaaaaagaagaacaacaagttacgtggaaatcatcacaagacagtagtttaacgccggaattaagcgcgaaagcaccaaaagaatatgatacatcacaacctttcacagacgcctctaacgatgttggctttgatgtgaatgagtatcatcatggaggtggcgacaatgcaagcaaaattaactaaaaatgagtttatagagtggttgaaaacttctgagggaaaacaattcaatgtggacttatggtatggatttcaatgctttgattatgccaatgctggttggaaagttttgtttggattacttctaaaaggtttaggtgcaaaagatattccgttcgctaacaacttcgacggattagctactgtataccaaaatacaccggacttcttagcacaacctggcgacatggtggtattcggtagcaactacggtgctggatatggtcacgttgcatgggtaattgaagcaactttagattacatcattgtatatgagcagaattggctaggcggtggctggactgacggaatcgaacaacccggctggggttgggaaaaagttacaagacgacaacatgcttatgatttccctatgtggtttatccgtccgaattttaaaagtgagacagcgccacgatcagttcaatctcctacacaagcacctaaaaaagaaacagctaagccacaacctaaagcagtagaacttaaaatcatcaaagatgtggttaaaggttatgacctacctaagcgtggtagtaaccctaaaggtatagttatacacaacgacgcagggagcaaaggggcgactgctgaagcatatcgtaacggattagtaaatgcacctttatcaagattagaagcgggcattgcgcatagttacgtatcaggcaacacagtttggcaagccttagatgaatcacaagtaggttggcataccgctaatcaaataggtaataaatattattacggtattgaagtatgtcaatcaatgggcgcagataacgcgacattcttaaaaaatgaacaggcaactttccaagaatgcgctagattgttgaaaaaatggggattaccagcaaacagaaatacaatcagattgcacaatgaatttacttcaacatcatgccctcatagaagttcggttttacacactggttttgacccagtaactcgcggtctattgccagaagacaagcggttgcaacttaaagactactttatcaagcagattagggcgtacatggatggtaaaataccggttgccactgtctctaatgagtcaagcgcttcaagtaatacagttaaaccagttgcaagtgcatggaaacgtaataaatatggtacttactacatggaagaaagtgctagattcacaaacggcaatcaaccaatcacagtaagaaaagtggggccattcttatcttgtccagtgggttatcagttccaacctggtgggtattgtgattatacagaagtgatgttacaagatggtcatgtttgggtaggatatacatgggaggggcaacgttattacttgcctattagaacatggaatggttctgccccacctaatcagatattaggtgacttatggggagaaatcagttagaatgacatagtcatgtctatttaagcaggtgcgttacatacctgctttctatttacatttaaagataaaatgtgctattattttactagaactttttaacatttctctcaagatttaaatgtagataacaggcaggtactacggtacttgcctatttttttatgcaaattttaaaaaacactttactaataaacatttgtttagtataattatatttgtaggttagttgatgacttacaaattatgtgtaaggaggtgaaaagcctcatgctagacataataaaaacacttctagaacatcaagtattggcagtactgataattccagaagtgttaaaacaacttagagaatggcatctcggctacctagaccgaaagccaaacaacaaagattaacattatgcttggagcctgatggctcctccttacacttatataatataatattatttggaggttttcaattatgacagaacaaatgtatttaatattgtttttattaagcctaccattgttattatttatcgggagaaagacacatttttattgtttagataaaaagaatggacgtagataatatgagtgattataaattaaaaataattgaattgatcaaaagtgatataacaggttaccaaattcacaaacaaactggcgtagcgcaatatgtaatttcacaattaaggcaaggaaagcgcgaagtagataacttaactttaaatacaactgaaaaactatacagttacgcacgacaagtgttataatataaatgtgaaatggtcattcttgaaatgactcggtcgctactggcacagaccgtttaaagtgtcaccacaacatgaactgagaattcatatgacgttgctgacgagcgacaaagctctgtgttcctgaatgggagtaggtttgtgtggtggtataatttagtaacagcatagactgtctatagcaaagttgccgaagagattctaaacgtatttataaatacgtggcccttgctagataaccgcatcttaactgatgcggttatttttatccccacacaaccaacaaaaccacaccacctattaatttaggagtgtggttgttttaatatgtgaagctaaaataactacaaatgataccatttttgataccattttgttgtaaaacagaaaaaataaggaaaataaaaaaggcaaaaaaacgcattaaatcaacgtttattgtctcatgaaatttaaatgtatataaatttca

SEQ ID NO: 18 - DNA, 18 bp, Artificial Sequence
  Synthetic Construct
aaagaagaacaataatat

SEQ ID NO: 19 - DNA, 18 bp, Artificial Sequence
  Synthetic Construct
aaagaagaacaataatat

SEQ ID NO: 20 - DNA, 18 bp, Artificial Sequence
  Synthetic Construct
taattattcccactcaat

SEQ ID NO: 21 - DNA, 18 bp, Artificial Sequence
  Synthetic Construct
taattattcccactcgat

SEQ ID NO: 22 - DNA, 15 bp, Artificial Sequence
  Synthetic Construct
tcccgccgtctccat

SEQ ID NO: 23 - DNA, 15 bp, Artificial Sequence
  Synthetic Construct
tcccgccgtctccat

SEQ ID NO: 24 - DNA, 15 bp, Artificial Sequence
  Synthetic Construct
tcccgccgtctccat

SEQ ID NO: 25 - DNA, 15 bp, Artificial Sequence
  Synthetic Construct
tcccgccgtctccat

SEQ ID NO: 26 - DNA, 17 bp, Artificial Sequence
  Synthetic Construct
ttatttagcaggaataa

SEQ ID NO: 27 - DNA, 17 bp, Artificial Sequence
  Synthetic Construct
ttatttagcaggaataa

SEQ ID NO: 28 - DNA, 17 bp, Artificial Sequence
  Synthetic Construct
ttatttagcaggaataa

SEQ ID NO: 29 - DNA, 22 bp, Artificial Sequence
  Synthetic Construct
gttttaccatcattcccggcat

SEQ ID NO: 30 - DNA, 21 bp, Artificial Sequence
  Synthetic Construct
attttacatcattcctggcat

SEQ ID NO: 31 - DNA, 21 bp, Artificial Sequence
  Synthetic Construct
atgccaggtatgatgtaaaac

SEQ ID NO: 32 - DNA, 21 bp, Artificial Sequence
  Synthetic Construct
gttttacatcattcctggcat

SEQ ID NO: 33 - DNA, 43 bp, Artificial Sequence
  Synthetic Construct
gttttaccatcattcccggcatgttttacatcattcctggcat

(19) United States
(12) Patent Application Publication
(10) Pub. No.: US 2020/0254035 A1
(43) Pub. Date: Aug. 13, 2020

(54) PHAGE AND TRANSDUCTION PARTICLES

(71) Applicant: SNIPR Biome ApS, Copenhagen (DK)

(72) Inventor: Jakob Krause HAABER, Copenhagen (DK)

(73) Assignee: SNIPR Biome ApS, Copenhagen (DK)

(21) Appl. No.: 16/839,164

(22) Filed: Apr. 3, 2020

Related U.S. Application Data
(63) Continuation of application No. 15/985,658, filed on May 21, 2018.

(30) Foreign Application Priority Data
Nov. 29, 2017 (GB) GB1719896.1
May 17, 2018 (GB) GB1808063.0

(51) Int. Cl. A61K 35/76; C12N 15/73; C12N 7/02; A61K 38/16; C07K 14/005

(52) U.S. Cl. CPC A61K 35/76; C12N 2795/10151; C12N 7/025; A61K 38/162; C07K 14/005; A61K 38/164; C12N 2795/00052; C12N 2795/10121; C12N 2795/10142; C12N 2795/10152; C12N 2310/20; C12N 2795/00042; C12N 2795/00032; C12N 2795/10132; C12N 15/73

(57) ABSTRACT

The invention relates to the production of phage and non-replicative transduction particles using DNAs (eg, plasmids and helper phage, mobile genetic elements (MGEs) or plasmids with chromosomally integrated helper phage genes), as well as the phage, helper phage, kits, compositions and methods involving these. The non-replicative transduction particles can be used to deliver antibacterial agents comprising a guided nuclease system.

### CROSS REFERENCE TO RELATED APPLICATION

This application claims priority benefit to United Kingdom Patent Application Nos. GB1719896.1 filed on Nov. 29, 2017 and GB1808063.0 filed on May 17, 2018, the contents of which are incorporated herein by reference in their entireties.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 786212000400SEQLIST.txt, date recorded: May 21, 2018, size: 71 KB).

### TECHNICAL FIELD

The invention relates to the production of phage using DNAs (eg, plasmids and helper phage, or plasmids with chromosomally integrated helper phage genes), as well as the phage, helper phage, kits, compositions and methods involving these.

### BACKGROUND

The use of helper phage to package phagemid DNA into phage virus particles is known. An example is the M13K07 helper phage, a derivative of M13, used in E coli host cells. Other examples are R408 and CM13.

### SUMMARY OF THE INVENTION

The invention relates to the production of phage and provides:—

In a First Configuration

A kit comprising
a) A first DNA; and
b) One or more second DNAs;

### Wherein

(i) the DNAs together comprise all phage structural protein genes required to produce a packaged phage particle comprising a copy of the first DNA;
(ii) the first DNA comprises none or at least one, but not all, of the genes; and wherein the one or more second DNAs comprise the remainder of the genes;
(iii) the first DNA comprises a phage packaging signal for producing the packaged phage particle; and
(iv) the second DNA is devoid of a nucleotide sequence (eg, a packaging signal) required for packaging the second DNA into phage particles;
wherein the DNAs are operable when co-existing in a host bacterium for producing packaged phage that comprise the first DNA, wherein the phage require the second DNA for replication thereof to produce further phage particles.
There is also provided
A method of producing phage, the method comprising expressing in a cell comprising the DNAs the phage protein genes, wherein packaged phage are produced that comprise the first DNA, wherein the phage require the second DNA for replication thereof to produce further phage particles.

In a Second Configuration

A population of helper phage, wherein the helper phage are capable of packaging first phage, wherein the first phage are different from the helper phage and the helper phage are incapable of self-replication.

In a Third Configuration

A composition comprising a population of first phage, wherein the first phage require helper phage according to the First Configuration for replication; and wherein less than [20%] of total phage comprised by the composition are such helper phage.

In a Fourth Configuration

A method of producing first phage, wherein the first phage require helper phage to replicate, the method comprising

(a) Providing DNA comprising a packaging signal;
(b) Introducing the DNA into a host bacterial cell;
(c) Wherein the host bacterial cell comprises helper phage or wherein helper phage are introduced into the bacterial cell simultaneously or sequentially with step (b);
(d) Wherein the helper phage are according to the invention;
(e) Causing or allowing the helper phage to produce phage proteins, wherein the packaging signal is recognised in the host cell, whereby first phage are produced using the proteins, the first phage packaging the DNA;
(f) Wherein helper phage replication in the host cell is inhibited or reduced, thereby limiting the availability of helper phage;
(g) Optionally lysing the host cell and obtaining the first phage;
(h) Thereby producing a composition comprising first phage which require the helper phage for replication, wherein propagation of first phage is prevented or reduced by the limitation of helper phage availability.

In a Fifth Configuration

A phage production system, for producing phage (eg, the first phage of any preceding claim) comprising a nucleotide sequence of interest (NSI-phage), the system comprising components (i) to (iii):—
(i) A first DNA;
(ii) A second DNA; and
(iii) a NSI-phage production factor (NPF) or an expressible nucleotide sequence that encodes a NPF;

### Wherein

a) The first DNA encodes a helper phage (eg, said first helper phage recited in any preceding claim);
b) The second DNA comprises the nucleotide sequence of interest (NSI);
c) When the system is comprised by a bacterial host cell, helper phage proteins are expressed from the first DNA to form phage that package the second DNA in the presence of the NPF, thereby producing NSI-phage;
d) The system is devoid of a helper phage production factor (HPF) that is required for forming phage that package the first DNA, or is devoid of an expressible nucleotide sequence that encodes a functional HPF; or the system comprises a nucleotide sequence that comprises or encodes a functional HPF, the system further comprising means for targeted inactivation in the host cell of the HPF sequence to eliminate or minimise production of helper phage comprising the first DNA; and
Whereby the system is capable of producing a product comprising a population of NSI-phage, wherein each NSI-phage requires a said helper phage for propagation, wherein the NSI-phage in the product are not mixed with helper phage or less than [20%] of total phage comprised by the product are said helper phage.
The invention also provides:

A composition for use in antibacterial treatment of bacteria, the composition comprising an engineered mobile genetic element (MGE) that is capable of being mobilised in a first bacterial host cell of a first species or strain, the cell comprising a first phage genome, wherein in the cell the MGE is mobilised using proteins encoded by the phage and replication of first is inhibited, wherein the MGE encodes an antibacterial agent or encodes a component of such an agent.

A nucleic acid vector comprising the MGE integrated therein, wherein the vector is capable of transferring the MGE or a copy thereof into a host bacterial cell.

A non-self replicative transduction particle comprising said MGE or vector of the invention.

A composition comprising a plurality of transduction particles, wherein each particle comprises a MGE or vector according to the invention, wherein the transduction particles are capable of transferring the MGEs, or nucleic acid encoding the agent or component, or copies thereof into target bacterial cells, wherein

(i) target cells are killed by the antibacterial agent;

(ii) growth or proliferation of target cells is reduced; or

(iii) target cells are sensitised to an antibiotic, whereby the antibiotic is toxic to the cells.

A composition comprising a plurality of non-self replicative transduction particles, wherein each particle comprises a MGE or plasmid according to the invention, wherein the transduction particles are capable of transferring the MGEs, or nucleic acid encoding the agent or component, or copies thereof into target bacterial cells, wherein the agent is a CRISPR/Cas system and the component comprises a nucleic acid encoding a crRNA or a guide RNA that is operable with a Cas in a target bacterial cell to guide the Cas to a target nucleic acid sequence of the cell to modify the sequence, whereby

(i) target cells are killed by the antibacterial agent;

(ii) growth or proliferation of target cells is reduced; or

(iii) target cells are sensitised to an antibiotic, whereby the antibiotic is toxic to the cells.

A method of producing a plurality of transduction particles, the method comprising combining the composition of the invention with host bacterial cells of said first species, wherein the cells comprise the first phage, allowing a plurality of said MGEs to be introduced into host cells and culturing the host cells under conditions in which first phage-encoded proteins are expressed and MGE copies are packaged by first phage proteins to produce a plurality of transduction particles, and optionally separating the transduction particles from cells and obtaining a plurality of transduction particles separated from cells.

A bacterial host cell comprising a first phage and a MGE, vector or particle of the invention, wherein the agent is toxic to cells of the same species as the host cell, and wherein the host cell has been engineered so that the agent is not toxic to the host cell.

A bacterial host cell comprising a first phage, wherein the cell is comprised by a kit, the kit further comprising a composition of the invention, wherein the agent is toxic to cells of the same species as the host cell, and wherein the host cell has been engineered so that the agent is not toxic to the host cell.

A bacterial host cell comprising a first phage and a MGE, vector or particle of the invention, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

A bacterial host cell comprising a first phage, wherein the cell is comprised by a kit, the kit further comprising a composition of the invention, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

A bacterial host cell comprising a MGE, vector or particle of the invention and nucleic acid under the control of one or more inducible promoters, wherein the nucleic acid encodes all structural proteins necessary to produce a transduction particle that packages a copy of the MGE or plasmid, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

A plasmid comprising
(a) A nucleotide sequence encoding an antibacterial agent or component thereof for expression in target bacterial cells;
(b) A constitutive promoter for controlling the expression of the agent or component;
(c) An optional terS nucleotide sequence;
(d) An origin of replication (ori); and
(e) A phage packaging sequence (optionally pac, cos or a homologue thereof); and
   the plasmid being devoid of
(f) All nucleotide sequences encoding phage structural proteins necessary for the production of a transduction particle (optionally a phage), or the plasmid being devoid of at least one of such sequences; and
(g) Optionally terL.

A bacterial host cell comprising the genome of a helper phage that is incapable of self-replication, optionally wherein the genome is present as a prophage, and a plasmid according to the invention, wherein the helper phage is operable to package copies of the plasmid in transduction particles, wherein the particles are capable of infecting bacterial target cells to which the antibacterial agent is toxic.

A method of making a plurality of transduction particles, the method comprising culturing a plurality of host cells according to the invention, optionally inducing a lytic cycle of the helper phage, and incubating the cells under conditions wherein transducing particles comprising packaged copies of the plasmid are created, and optionally separating the particles from the cells to obtain a plurality of transduction particles.

A plurality of transduction particles obtainable by the method of the invention for use in medicine, eg, for treating or preventing an infection of a human or animal subject by target bacterial cells, wherein transducing particles are administered to the subject for infecting target cells and killing the cells using the antibacterial agent.

A method of making a plurality of transduction particles, the method comprising
(a) Producing host cells whose genomes comprise nucleic acid encoding structural proteins necessary to produce transduction particles that can package first DNA, wherein the genomes are devoid of a phage packaging signal, wherein the expression of the proteins is under the control of inducible promoter(s);
(b) Producing first DNA encoding an antibacterial agent or a component thereof, wherein the DNA comprises a phage packaging signal;
(c) Introducing the DNA into the host cells;
(d) Inducing production of the structural proteins in host cells, whereby transduction particles are produced that package the DNA;
(e) Optionally isolating a plurality of the transduction particles; and
(f) Optionally formulating the particles into a pharmaceutical composition for administration to a human or animal for medical use.

A plurality of transduction particles obtainable by the method.

### BRIEF DESCRIPTION OF THE FIGURES

FIG. 1 shows a genetic map of P2 genome.

FIG. 2 shows an exemplary saPI system (SaPIbov1).

FIG. 3 shows exemplary SaPIs.

### DETAILED DESCRIPTION

The invention relates to the production of phage using DNAs (eg, plasmids with helper phage), as well as the phage, helper phage, compositions and methods involving these. The invention finds utility, for example, for containing phage in environments ex vivo and in vivo, reducing the risk of acquisition of antibiotic resistance or other genes by phage, as well as controlling dosing of phage in an environment. The contamination of useful phage populations by helper phage may in examples also be restricted or eliminated, thereby controlling phage propagation and enhancing the proportion of desired phage in phage compositions, such as medicaments, herbicides and other agents where phage may usefully be used. Thus, the invention provides the following embodiments.

A kit comprising

a) A first DNA; and
b) One or more second DNAs;

### Wherein

(i) the DNAs together comprise all phage structural protein genes required to produce a packaged phage particle comprising a copy of the first DNA;
(ii) the first DNA comprises none or at least one, but not all, of the genes; and wherein the one or more second DNAs comprise the remainder of the genes;
(iii) the first DNA comprises a phage packaging signal for producing the packaged phage particle; and
(iv) the second DNA is devoid of a nucleotide sequence required for packaging the second DNA into phage particles;
wherein the DNAs are operable when co-existing in a host bacterium for producing packaged phage that comprise the first DNA, wherein the phage require the second DNA for replication thereof to produce further phage particles.

For example the second DNA is devoid of a packaging signal for packaging second DNA. Additionally or alternatively, the second DNA is devoid of a nucleotide sequence required for replication of helper phage. Optionally, the nucleotide sequence encodes a sigma factor or comprises a sigma factor recognition site, a DNA polymerisation recognition site, or a promoter of a gene required for helper phage DNA replication when the second DNA is comprised by a helper prophage.

In an example, the second DNA is comprised by an M13 or M13-based helper phage. M13 encodes the following proteins required for phage packaging:—

a. pIII: host recognition
b. pV: coat protein
c. pVII, pVIII, pIX: membrane proteins
d. pI, pIV, pXI: Channel for translocating the phage to the extracellular space.

In this example, the second DNA is devoid of one or more of the genes coding for these proteins, eg, is devoid of a gene encoding pIII, a gene encoding pV, a gene encoding pVII, a gene encoding pVIII, a gene encoding pIX, a gene encoding pI, a gene encoding pIV and/or a gene encoding XI.

In an embodiment, the phage particle of (i) is capable of infecting a target bacterium, the phage comprising a nucleotide sequence of interest (NSI) that is capable of expressing a protein or RNA in the target bacterium, or wherein the NSI comprises a regulatory element that is operable in the target bacterium. In an example, the NSI is capable of recombination with the target cell chromosome or an episome comprised by the target cell to modify the chromosome or episome. Optionally, this is carried out in a method wherein the chromosome or episome is cut (eg, at a predetermined site using a guided nuclease, such as a Cas, TALEN, zinc finger or meganuclease; or a restriction endonuclease) and simultaneously or sequentially the cell is infected by a phage particle that comprises the first DNA, wherein the DNA is introduced into the cell and the NSI or a sequence thereof is introduced into the chromosome or episome at or adjacent the cut site. In an example the first DNA comprises one or more components of a CRISPR/Cas system operable to perform the cutting (eg, comprising at least a nucleotide sequence encoding a guide RNA or crRNA for targeting the site to be cut) and further comprising the NSI.

In an embodiment, the presence in the target bacterium of the NSI or its encoded protein or RNA mediates target cell killing, or downregulation of growth or propagation of target cells, or mediates switching off of expression of one or more RNA or proteins encoded by the target cell genome, or downregulation thereof.

In an embodiment, the presence in the target bacterium of the NSI or its encoded protein or RNA mediates upregulation of growth or propagation of the target cell, or mediates switching on of expression of one or more RNA or proteins encoded by the target cell genome, or upregulation thereof.

In an embodiment, the NSI encodes a component of a CRISPR/Cas system that is toxic to the target bacterium.

In an embodiment, the DNA is a first DNA as defined in any preceding paragraph.

In an embodiment, the first DNA is comprised by a vector (eg, a plasmid or shuttle vector).

In an embodiment, the second DNA is comprised by a vector (eg, a plasmid or shuttle vector), helper phage (eg, a helper phagemid) or is integrated in the genome of a host bacterial cell.

An embodiment provides a bacterial cell comprising the first and second DNAs. Optionally, the cell is devoid of a functional CRISPR/Cas system before transfer therein of a first DNA, eg, a first DNA comprising a component of a CRISPR/Cas system that is toxic to the target bacterium. An embodiment provides an antibacterial composition comprising a plurality of cells, wherein each cell is optionally according to this paragraph, for administration to a human or animal subject for medical use.

A method of producing phage is provided, the method comprising expressing in a host bacterial cell the phage protein genes, wherein packaged phage are produced that comprise the first DNA, wherein the phage require the second DNA for replication thereof to produce further phage particles. Optionally, the method comprises isolating the phage particles.

A composition comprising a population of phage particles obtainable by the method is provided for administration to a human or animal subject for treating an infection of target bacterial cells, wherein the phage are capable of infecting and killing the target cells.

A method of treating an environment ex vivo, the method comprising exposing the environment to a population of phage particles obtainable by the method is provided, wherein the environment comprises target bacteria and the phage infect and kill the target bacteria. In an example thje subject is further administered an agent simultaneously or sequentially with the phage administration. In an example, the agent is a herbicide, pesticide, insecticide, plant fertilizer or cleaning agent.

Optionally, the method is for containing the treatment in the environment.

Optionally, the method is for controlling the dosing of the phage treatment in the environment.

Optionally, the method is for reducing the risk of acquisition of foreign gene sequence(s) by the phage in the environment.

A method of treating an infection of target bacteria in a human or animal subject is provided, the method comprising exposing the bacteria to a population of phage particles obtainable by the production method, wherein the phage infect and kill the target bacteria.

Optionally, the method for treating is for containing the treatment in the subject.

Optionally, the method for treating is for containing the treatment in the environment in which the subject exists.

Optionally, the method for treating is for controlling the dosing of the phage treatment in the subject.

Optionally, the method for treating is for reducing the risk of acquisition of foreign gene sequence(s) by the phage in the subject.

Optionally, the method for treating is for reducing the risk of acquisition of foreign gene sequence(s) by the phage in the environment in which the subject exists.

Optionally, target bacteria herein are comprised by a microbiome of the subject, eg, a gut microbiome. Altertnatively, the microbiome is a skin, scalp, hair, eye, ear, oral, throat, lung, blood, rectal, anal, vaginal, scrotal, penile, nasal or tongue microbiome.

In an example thje subject is further administered a medicament simultaneously or sequentially with the phage administration. In an example, the medicament is an antibiotic, antibody, immune checkpoint inhibitor (eg, an anti-PD-1, anti-PD-L1 or anti-CTLA4 antibody), adoptive cell therapy (eg, CAR-T therapy) or a vaccine.

In an example, the invention employs helper phage for packaging the phage nucleic acid of interest. Thus, the invention provides the following illustrative Aspects:—

1. A population of helper phage, wherein the helper phage are capable of packaging first phage nucleic acid to produce first phage particles, wherein the first phage are different from the helper phage and the helper phage are incapable themselves of producing helper phage particles.
2. A composition comprising a population of first phage, wherein the first phage require helper phage according to Aspect 1 for replication of first phage particles; and optionally wherein less than 20, 15, 10, 5, 4, 3, 2, 1, 0.5, 0.4, 0.2 or 0.1% of total phage particles comprised by the composition are particles of such helper phage.

In an example, the population comprises at least 10³, 10⁴, 10⁵ or 10⁶ phage particles, as indicated a transduction assay, for example. To have a measure of the first phage concentration, for example, one can perform a standard transduction assay when the first phage genome contains an antibiotic marker. Thus, in this case the first phage are capable of infecting target bacteria and in a sample of 1 ml the population comprises at least 10³, 10⁴, 10⁵ or 10⁶ transducing particles, which can be determined by infecting susceptible bacteria at a multiplicity of infection <0.1 and determining the number of infected cells by plating on a selective agar plate corresponding to the antibiotic marker in vitro at 20 to 37 degrees centigrade, eg, at 20 or 37 degrees centrigrade.

Optionally at least 99.9, 99.8, 99.7, 99.6, 99.5, 99.4, 99.3, 99.2, 99.1, 90, 85, 80, 70, 60, 50 or 40% of total phage particles comprised by the composition are particles of first phage.

In an example, the first phage genome comprises an f1 origin of replication.

In an example, the helper phage are E coli phage. In an example, the first phage are E coli, C dificile, Streptococcus, Klebsiella, Pseudomonas, Acitenobacter, Enterobacteracea, Firmicutes or Bacteroidetes phage. In an example, the helper phage are engineered M13 phage.

In an example, the first phage genome comprises a phagemid, wherein the phagemid comprises a packaging signal for packaging first phage particles in the presence of the helper phage.

The first phage particles may contain a nucleotide sequence of interest (NSI), eg, as defined herein, such as a NSI that encodes a component of a CRISPR/Cas system operable in target bacteria that can be infected by the first phage particles. Once inside the target bacteria, the first phage DNA is incapable of being packaged to form first phage particles in the absence of the helper phage. This usefully contains the activity of the first phage genome and its encoded products (proteins and/or nucleic acid), as well as limits or controls dosing of the NSI and its encoded products in an environment comprising the target bacteria that have been exposed to the first phage. This is useful, for example to control the medical treatment of an environment comprised by a human or animal subject, plant or other environment (eg, soil or a foodstuff or food ingredient).

3. The helper phage or composition of any preceding Aspect, wherein the genome of each first phage is devoid of genes encoding first phage structural proteins.
4. The composition of Aspect 2 or 3, wherein the composition comprises helper phage DNA.
5. The composition of Aspect 4, wherein the DNA comprises helper DNA fragments.
6. The helper phage or composition of any one preceding Aspect, wherein the helper phage are in the form of prophage.
Thus, the prophage is integrated in the chromosome of a host cell.
Examples of phage structural proteins are phage coat proteins, collar proteins and phage tail fibre proteins.
7. The composition of any one of Aspects 2 or 3, wherein the composition comprises no helper phage DNA comprising a sequence of 20 contiguous nucleotides or more, eg, no helper phage DNA.
This can be determined, for example, using DNA probes (designed on the basis of the known helper phage genome sequence) with PCR, as is conventional. In an example, the composition may comprise residual helper prophage DNA, but essentially otherwise is devoid of helper DNA.
8. The composition of any one of Aspects 2 to 5 and 7, wherein the helper phage are capable of infecting host bacteria and the composition does not comprise host bacteria.
9. The composition of any one of Aspects 2 to 8, wherein the composition is a lysate of host bacterial cells, wherein the lysate comprises helper prophage DNA, eg, such DNA comprises 20 contiguous nucleotides or more of helper phage DNA.
10. The composition of any one of Aspects 2 to 8, wherein the composition is a lysate of host bacterial cells, wherein the lysate has been processed (eg, filtered) to remove all or some helper phage DNA; or the composition is a lysate of host bacterial cells that is devoid of cellular material.
11. The composition of any one of Aspects 2 to 10, wherein the composition does not comprise helper phage particles.
12. The composition of any one of Aspects 2 to 11, wherein at least 95% (eg, 100%) of phage particles comprised by the composition are first phage particles.
In another embodiment, the composition comprises second phage particles, wherein the second phage are different from the first phage and are not helper phage.
13. The composition of any one of Aspects 2 to 12, wherein the population comprises at least 10³, 10⁴, 10⁵ or 10⁶ phage particles, as indicated in a transduction assay.
14. The helper phage or composition of any preceding Aspect, wherein the first phage are capable of replicating in host bacteria in the presence of the helper phage (eg, helper prophage), wherein the first phage comprise antibacterial means for killing target bacteria of a first strain or species, wherein the target bacteria are of a different strain or species and the antibacterial means is not operable to kill the target bacteria.
15. A composition comprising a population of phage, the population comprising
(a) A first sub-population of first phage that require a helper phage for packaging the first phage;
(b) A second sub-population of phage comprising the helper phage, wherein the helper phage are as recited in any preceding Aspect.
   16. The helper phage or composition of any preceding Aspect, wherein the helper phage are phagemids.
   17. A composition comprising
(a) A population of helper phage as recited in any preceding Aspect; and
(b) A population of nucleic acid vectors comprising vector DNA that comprises a first phage packaging signal;
(c) wherein the helper phage are capable of packaging the vector DNA to produce first phage.
   18. The composition of Aspect 17, wherein the vectors are phage.
   19. The composition of Aspect 17, wherein the vectors are plasmids or phagemids.
   20. The composition of Aspect 19, the vectors are shuttle vectors (eg, pUC vectors) that can be replicated in first bacteria, wherein the vectors can further be replicated and packaged into first phage in second bacteria (host bacteria) in the presence of the helper phage, wherein the first bacteria are of a strain or species that is different to the strain or species of the host bacteria.
   21. The composition of Aspect 21, wherein the first phage are capable of infecting third bacteria of a strain or species that is different to the second (and optionally also the first) bacteria.
   22. The composition of any one of Aspects 17 to 21, wherein the first phage are capable of replicating in host bacteria in the presence of the helper phage (eg, helper prophage), wherein the first phage comprise antibacterial means for killing target bacteria of a first strain or species, wherein the host bacteria are of a different strain or species and the antibacterial means is not operable to kill the host bacteria.
   23. The helper phage or composition of any preceding Aspect, wherein the genome is devoid of a packaging signal (eg, SEQ ID NO:1 below), wherein the helper phage are incapable of self-replication.
   24. The helper phage or composition of Aspect 24, wherein the signal is a pac or cos sequence.
   25. The helper phage or composition of any preceding Aspect, wherein the helper phage genome is capable of replication in a host cell.
   Thus, the genome is capable of nucleic acid replication but not packaging of helper phage.
   26. The helper phage or composition of any one of Aspects 1 to 24, wherein the genome is devoid of a nucleotide sequence required for production of helper phage particles.
   27. The helper phage or composition of Aspect 26, wherein the nucleotide sequence enodes a sigma factor (eg, sigma-70) or comprises a sigma factor recognition site, a DNA polymerisation recognition site, or a promoter of a gene required for helper phage DNA replication.
   28. The helper phage or composition of any preceding Aspect, wherein the helper phage are temperate phage.
   29. The helper phage or composition of any one of Aspects 1 to 27, wherein the helper phage are lytic phage.
   30. The helper phage or composition of any preceding Aspect, wherein the first phage are capable of infecting target bacteria, the first phage comprising a nucleotide sequence of interest (NSI) that is capable of expressing a protein or RNA (eg, gRNA or crRNA) in target bacteria, or wherein the NSI comprises a regulatory element that is operable in target bacteria.
   31. The helper phage or composition of Aspect 30, wherein the presence in target bacteria of the NSI or its encoded protein or RNA mediates target cell killing, or downregulation of growth or propagation of target cells, or mediates switching off of expression of one or more RNA or proteins encoded by the target cell genomes, or downregulation thereof.
   32. The helper phage or composition of Aspect 30, wherein the presence in target bacteria of the NSI or its encoded protein or RNA mediates upregulation of growth or propagation of target cells, or mediates switching on of expression of one or more RNA or proteins encoded by the target cell genomes, or upregulation thereof.
   33. An antibacterial composition according to any one of Aspects 2 to 32, wherein the first phage are capable of infecting target bacteria and each first phage comprises engineered antibacterial means for killing target bacteria.
   By use of the term “engineered” it will be readily apparent to the skilled addressee that the relevant means has been introduced and is not naturally-occurring in the phage. For example, the means is recombinant, artificial or synthetic.
   34. The composition of Aspect 14, 22 or 33, wherein the antibacterial means comprises one or more components of a CRISPR/Cas system.
   35. The composition of claim 34, wherein the component(s) comprise (i) a DNA sequence encoding a guide RNA (eg, a single guide RNA) or comprising a CRISPR array for producing guide RNA, wherein the guide RNA is capable of targeting the genome of target bacteria; (ii) a Cas nuclease-encoding DNA sequence; and/or (iii) a DNA sequence encoding one or more components of Cascade.
   In an example, a Cas herein is a Cas9. In an example, a Cas herein is a Cas3. The Cas may be identical to a Cas encoded by the target bacteria.
   36. The composition of any one of Aspects 14, 22 or 33 to 35, wherein the antibacterial means comprises a nucleic acid encoding a guided nuclease, such as a Cas nuclease, TALEN, zinc finger nuclease or meganuclease.
   37. The helper phage or composition of any preceding Aspect, wherein the helper phage is for use in medicine practised on a human or animal subject, or the composition is a pharmaceutical composition for use in medicine practised on a human or animal subject.
   In an example, the animal is a livestock or companion pet animal (eg, a cow, pig, goat, sheep, horse, dog, cat or rabbit). In an example, the animal is an insect (an insect at any stage of its lifecycle, eg, egg, larva or pupa). In an example, the animal is a protozoan. In an example, the animal is a cephalopod.
   38. The composition of any one of Aspects 2 to 36, wherein the composition is a herbicide, pesticide, food or beverage processing agent, food or beverage additive, petrochemical or fuel processing agent, water purifying agent, cosmetic additive, detergent additive or environmental (eg, soil) additive or cleaning agent.
   39. The helper phage or composition of any one of Aspects 1 to 37 for use in a contained method of treating a disease or condition of a human or animal subject, wherein the disease or condition is mediated by the target bacteria and the target bacteria are comprised by the subject, the method comprising administering the composition to the subject, whereby the target bacteria are exposed to the antibacterial means and killed and propagation of the first phage is contained.
   The inability of the first phage to self-replicate and to require helper phage or second DNA to do this usefully provides containment in the location (eg, gut) of action of the composition and/or in the environment of the subject, eg, when exposed to secretions such as urine and faeces of the subject that otherwise may contain replicated first phage. Inability of the helper phage or second DNA to self-package limits availability of factors required by the first phage to form packaged particles, hence providing containment by limiting first phage propagation. This may be useful, for example, to contain an antibacterial activity provided by the first phage, such as a CRISPR/Cas killing principle.
   40. A bacterial cell or a plurality of bacterial cells comprising the helper phage or composition of any preceding Aspect, wherein the first phage are capable of replication in the presence of the helper phage in the cell.
   The cell may, for example, act as a carrier for the genome of the first phage, wherein the first phage DNA is capable of horizontal transfer from the carrier to the target bacteria once the carrier bacteria have been administered to an environment to be treated, eg, a soil or a human gut or other environment described herein. In an example, the environment is comprised by a human or animal subject and the carrier are commensal or probiotic in the subject. For example the carrier bacteria are Lactobacillus (eg, L reuteri or L lactis), E coli or Streptococcus (eg, S thermophiles) bacteria. The horizontal transfer can be transfer of a plasmid (such as a conjugative plasmid) to the target bacteria or first phage infection of the target bacteria, wherein the first phage have been prior packaged in the carrier. The use of a carrier is useful too for oral administration or other routes where the carrier can provide protection for the phage, helper or composition from the acid stomach or other harsh environments in the subject. Furthermore, the carrier can be formulated into a beverage, for example, a probiotic drink, eg, an adapted Yakult (trademark), Actimel (trademark), Kevita (trademark), Activia (trademark), Jarrow (trademark) or similar drink for human consumption.
   41. The cell(s) of Aspect 40 for administration to a human or animal subject for medical use, comprising killing target bacteria using first phage, wherein the target bacteria mediate as disease or condition in the subject.
   In an example, when the subject is a human, the subject is not an embryo.
   42. The cell(s) of Aspect 41, wherein the cell(s) comprises helper phage and is symbiotic or probiotic in the subject.
   43. A method of killing target bacteria in an environment, optionally wherein the method is not practised on a human or animal body, wherein the method comprises exposing the environment to the cell(s) according to Aspect 42, or a composition obtained or obtainable by the method of any one of Aspects 57 to 65, wherein the environment is or has been exposed to first phage or said vectors to produce first phage in the presence of the helper phage, wherein the first phage are capable of replication in the environment and kill target bacteria.
   44. The cell(s) or method of any one of Aspects 40 to 43, wherein the cell is an E coli, Lactobacillus (eg, L lactis or retueri) or Streptococcus (eg, thermophilus) cell.
   45. The cell(s) or method of Aspects 40 to 44 wherein the subject is administered or has been administered a cell comprising first phage.
   46. The composition of any one of Aspects 2 to 45 in combination with a target bacterial cell wherein the first phage are capable of infecting the target bacterial cell.
   47. Use of the helper phage, composition or cell(s) of any one of Aspects 1 to 42 and 44 to 46, or a composition obtained or obtainable by the method of any one of Aspects 57 to 65, in the manufacture of an antibacterial agent that kills target bacteria, for containment of the antibacterial in an environment, eg, containment ex vivo; or containment in a human or animal subject comprising the environment.
   48. Use of the helper phage, composition or cell(s) of any one of Aspects 1 to 42 and 44 to 46, or a composition obtained or obtainable by the method of any one of Aspects 57 to 65, in the manufacture of an antibacterial agent that kills the target bacteria, for reducing the risk of acquisition by the first phage of foreign genes.
   For example, this is useful for reducing the risk of antibiotic resistance genes by the phage, such as when the phage are in the presence of other phage or plasmids in the environment.
   49. Use of the helper phage, composition or cell(s) of any one of Aspects 1 to 42 and 44 to 46, or a composition obtained or obtainable by the method of any one of Aspects 57 to 65, in the manufacture of an antibacterial agent that kills the target bacteria, for reducing the risk of acquisition by the first phage of one or more antibiotic resistance genes.
   50. A method of reducing the risk of acquisition by first phage of foreign genes, the method comprising
(a) Providing the composition of any one of Aspects 2 to 42 and 44 to 46, or a composition obtained or obtainable by the method of any one of Aspects 57 to 65; and
(b) Exposing target bacteria to the composition, wherein the first phage infect the target bacteria;
(c) wherein the helper phage are incapable of self-replication and propagation of first phage is thereby limited, wherein propagation of first phage is prevented or reduced, thereby reducing the risk of acquisition of first phage of foreign genes (eg, antibiotic resistance genes).
   51. A method of containing an antibacterial activity in an environment (e.g., ex vivo), the method comprising
(a) Providing an antibacterial composition according to any one of Aspects 2 to 42 and 44 to 46, or a composition obtained or obtainable by the method of any one of Aspects 57 to 65; and
(b) Exposing target bacteria in the environment to the composition, wherein the bacteria are exposed to the first phage and antibacterial means and are killed;
(c) wherein the helper phage are incapable of self-replication and propagation of first phage is thereby limited, wherein propagation of first phage is prevented or reduced, thereby containing the antibacterial activity.
   52. A method of controlling the dosing of first phage in an environment (e.g., ex vivo), the method comprising
(a) Providing an antibacterial composition according to any one of Aspects 2 to 42 and 44 to 46, or a composition obtained or obtainable by the method of any one of Aspects 57 to 65; and
(b) Exposing target bacteria in the environment to the composition, wherein the bacteria are infected by first phage;
(c) wherein the helper phage are incapable of self-replication and propagation of first phage is thereby limited, wherein propagation of first phage is prevented or reduced, thereby controlling dosing of first phage in the environment.
   53. The method of any one of Aspects 43 to 45, 51 and 52, or the use of Aspect 47, wherein the environment is a human or animal microbiome, e.g., a gut microbiome.
   54. The method of any one of Aspects 43 to 45, 51 and 52, or the use of Aspect 47, wherein the environment is a microbiome of soil; a plant, part of a part (e.g., a leaf, fruit, vegetable or flower) or plant product (e.g., pulp); water; a waterway; a fluid; a foodstuff or ingredient thereof; a beverage or ingredient thereof; a medical device; a cosmetic; a detergent; blood; a bodily fluid; a medical apparatus; an industrial apparatus; an oil rig; a petrochemical processing, storage or transport apparatus; a vehicle or a container.
   55. The method of any one of Aspects 43 to 45, 51 and 52, or the use of Aspect 47, wherein the environment is an ex vivo bodily fluid (e.g., urine, blood, blood product, sweat, tears, sputum or spit), bodily solid (e.g., faeces) or tissue of a human or animal subject that has been administered the composition.
   56. The method of any one of Aspects 43 to 45, 51 and 52, or the use of Aspect 47, wherein the environment is an in vivo bodily fluid (e.g., urine, blood, blood product, sweat, tears, sputum or spit), bodily solid (e.g., faeces) or tissue of a human or animal subject that has been administered the composition.
   57. A method of producing first phage, wherein the first phage require helper phage to replicate, the method comprising
(a) Providing DNA comprising a packaging signal;
(b) Introducing the DNA into a host bacterial cell;
(c) Wherein the host bacterial cell comprises helper phage or wherein helper phage are introduced into the bacterial cell simultaneously or sequentially with step (b);
(d) Wherein the helper phage are according to any preceding Aspect;
(e) Causing or allowing the helper phage to produce phage coat proteins, wherein the packaging signal is recognised in the host cell, whereby first phage are produced using the proteins, the first phage packaging the DNA;
(f) Wherein helper phage particle production in the host cell is inhibited or reduced, thereby limiting the availability of helper phage particles;
(g) Optionally lysing the host cell and obtaining the first phage;
(h) Thereby producing a composition comprising first phage which require the helper phage for replication, wherein further production of first phage particles is prevented or reduced by the limitation of helper phage availability in the composition.
   In an embodiment, the DNA is comprised by a phagemid or cloning vector (eg, a shuttle vector, eg, a pUC vector).
   There may be a modest amount of helper phage DNA replication to enable first phage protein production efficiently, or should replication of helper phage DNA may be eliminated totally eliminated.
   58. The method of Aspect 57, wherein in (c) the helper phage are prophage integrated in the bacterial cell chromosome.
   59. The method of Aspect 59, wherein (e) comprises inducing replication of helper phage DNA and/or expression of the proteins, eg, using UV, mitomycin.
   60. The method of any one of Aspects 57 to 59, wherein (g) comprises further separating the first phage from cellular material or helper phage DNA.
   61. The method of any one of Aspects 57 to 60, wherein the composition comprises a population of first phage particles, wherein the composition does not comprise helper phage DNA and/or particles.
   62. The method of any one of Aspects 57 to 61, wherein the DNA of (a) comprises engineered antibacterial means for killing target bacteria.
   63. The method of Aspect 62, wherein the antibacterial means comprises one or more components of a CRISPR/Cas system.
   64. The method of Aspect 63, wherein the component(s) comprise (i) a DNA sequence encoding a guide RNA (eg, a single guide RNA) or comprising a CRISPR array for producing guide RNA, wherein the guide RNA is capable of targeting the genome of target bacteria; (ii) a Cas (eg, Cas9, Cas3, Cpf1, CasX or CasY) nuclease-encoding DNA sequence; and/or (iii) a DNA sequence encoding one or more components of Cascade (eg, CasA).
   65. The method of any one of Aspects 62 to 64, wherein the antibacterial means comprises a nucleic acid encoding a guided nuclease, such as a Cas nuclease, TALEN, zinc finger nuclease or meganuclease.
   66. The helper phage, composition or cell(s) of any one of Aspects 1 to 42 and 44 to 46, or a composition obtained or obtainable by the method of any one of Aspects 57 to 65, for antibacterial treatment of target bacteria in a human or animal subject whereby the antibacterial treatment is contained in the subject.
   67. The helper phage, composition or cell(s) of any one of Aspects 1 to 42 and 44 to 46, or a composition obtained or obtainable by the method of any one of Aspects 57 to 65, for antibacterial treatment of target bacteria in a gut of a human or animal subject whereby the antibacterial activity in one or more bodily excretions of the subject is reduced.
   This is useful as a safety measure to reduce or eliminate first phage activity outside the subject.
   68. The helper phage, composition or cell(s) of Aspect 67, wherein the antibacterial activity in one or more bodily excretions of the subject is eliminated.
   69. The helper phage, composition or cell(s) of any one of Aspects 1 to 42 and 44 to 46, or a composition obtained or obtainable by the method of any one of Aspects 57 to 65, for controlling the dosing of antibacterial treatment of target bacteria in a human or animal subject, eg, in the gut of the subject.
   Usefully, propagation of the first phage is restricted or eliminated, so dosing in the subject can be controlled, or even pre-determined within a narrow expected range. This is useful, for example, for medicaments comprising the first phage or composition, and may be aid approval of such medicines before FDA and similar authorities.
   Alternatively, the dosing is dosing of an environment, such as soil etc disclosed herein, wherein limitation of the first phage or composition activity is also desirable to limit spread of activities in natural and other terrains.
   70. The helper phage, composition or cell(s) of any one of Aspects 1 to 42 and 44 to 46, or a composition obtained or obtainable by the method of any one of Aspects 57 to 65, for fixing the dosing of antibacterial treatment of target bacteria in a human or animal subject, eg, in the gut of the subject.
   71. A phage production system, for producing phage (eg, the first phage of any preceding Aspect) comprising a nucleotide sequence of interest (NSI-phage), the system comprising components (i) to (iii):—
(a) A first DNA;
(b) A second DNA; and
(c) a NSI-phage production factor (NPF) or an expressible nucleotide sequence that encodes a NPF;
   Wherein
(d) The first DNA encodes a helper phage (eg, said first helper phage recited in any preceding Aspect);
(e) The second DNA comprises the nucleotide sequence of interest (NSI);
(f) When the system is comprised by a bacterial host cell, helper phage proteins are expressed from the first DNA to form phage that package the second DNA in the presence of the NPF, thereby producing NSI-phage; and
(g) The system is devoid of a helper phage production factor (HPF) that is required for forming helper phage particles that package the first DNA, or is devoid of an expressible nucleotide sequence that encodes a functional HPF; or the system comprises a nucleotide sequence that comprises or encodes a functional HPF, the system further comprising means for targeted inactivation in the host cell of the HPF sequence to eliminate or minimise production of helper phage comprising the first DNA;
   Whereby the system is capable of producing a product comprising a population of NSI-phage, wherein each NSI-phage requires a said helper phage for propagation, optionally wherein the NSI-phage in the product are not mixed with helper phage or less than 20% of total phage comprised by the product are said helper phage.
   The invention includes within its concept relatively low level of helper phage particle production if there is a residual capability of helper phage to replicate to produce particles, such as for example in the case that a helper phage packaging signal or other HPF nucleotide sequence in the helper phage genome is mutated (eg, by deletion, substitution or addition of nucleotides therein) to knock down the ability to form phage particles. Preferably, there is no production of helper phage particles, such as by deleting all or part of the sequence from the helper phage genome or inactivating the sequence.
   72. A method of producing first phage, wherein the first phage require helper phage to replicate, the method comprising
(a) Providing in host cells the system of Aspect 71;
(b) Causing or allowing the helper phage proteins to be produced, whereby the second DNA is packaged to produce first phage; and
(c) Optionally lysing the host cells and obtaining a composition comprising first phage.
   73. The method of Aspect 72, wherein step (c) comprises separating the first phage from cellular material.
   74. The method of Aspect 72 or 73, wherein the composition comprises a population of first phage, wherein less than 20, 10, 5, 4, 3, 2, 1, 0.5 or 0.1% of total phage comprised by the composition are helper phage.
   75. The method of any one of Aspects 72 to 74, wherein the second DNA comprises engineered antibacterial means for killing target bacteria.
   76. The method of Aspect 75, wherein the antibacterial means comprises one or more components of a CRISPR/Cas system.
   77. The method of Aspect 76 wherein the component(s) comprise (i) a DNA sequence encoding a guide RNA (eg, a single guide RNA) or comprising a CRISPR array for producing guide RNA, wherein the guide RNA is capable of targeting the genome of target bacteria; (ii) a Cas nuclease-encoding DNA sequence; and/or (iii) a DNA sequence encoding one or more components of Cascade.
   78. The method of any one of Aspects 75 to 77, wherein the antibacterial means comprises a nucleic acid encoding a guided nuclease, such as a Cas nuclease, TALEN, zinc finger nuclease or meganuclease
   79. The system of method of any one of Aspects 71 to 78, wherein the first phage are capable of infecting target bacteria, the NSI being capable of expressing a protein or RNA in target bacteria, or wherein the NSI comprises a regulatory element that is operable in target bacteria.
   80. The system or method of Aspect 79, wherein the presence in target bacteria of the NSI or its encoded protein or RNA mediates target cell killing, or downregulation of growth or propagation of target cells, or mediates switching off of expression of one or more RNA or proteins encoded by the target cell genomes, or downregulation thereof.
   81. The system or method of Aspect 79, wherein the presence in target bacteria of the NSI or its encoded protein or RNA mediates upregulation of growth or propagation of target cells, or mediates switching on of expression of one or more RNA or proteins encoded by the target cell genomes, or upregulation thereof.
   82. The system of method of any one of Aspects 71 to 81, wherein each of the NPF and HPF is a packaging signal, eg, SEQ ID NO:1 or a sequence that is at least 70, 80, 90, 95, 96, 97, 98 or 99% identical thereto, or is a homologue from a different species.
   83. The system of method of Aspect 82, wherein each signal is a pac or cos sequence, or is a homologue.
   84. The system of method of any one of Aspects 71 to 81, wherein the HPF is a nucleotide sequence required for replication of helper phage.
   85. The system of method of any one of Aspects 71 to 81, wherein the HPF enodes a sigma factor (eg, sigma-70) or comprises a sigma factor recognition site, a DNA polymerisation recognition site, or a promoter of a gene required for helper phage DNA replication, a helper phage integrase, a helper phage excissionase or a helper phage origin of replication,
   86. A composition comprising a population of first phage obtainable by the method of any one of Aspects 72 to 85, wherein the genome of each first phage is devoid of genes encoding phage proteins.
   87. The composition of Aspect 86, wherein the first phage comprise antibacterial means as recited in any one of Aspects 75 to 78.
   88. The composition of Aspect 87, comprising DNA identical to the first DNA or fragments thereof.
   89. The composition of Aspect 88, wherein the DNA of the composition is identical to the first DNA and is devoid of a helper phage packaging signal.
   90. The composition of any one of Aspects 86 to 89 for antibacterial treatment of target bacteria in a human or animal subject whereby the antibacterial treatment is contained in the subject.
   91. The composition of any one of Aspects 86 to 89 for antibacterial treatment of target bacteria in a gut of a human or animal subject whereby the antibacterial activity in one or more bodily excretions of the subject is reduced.
   92. The composition of Aspect 91, wherein the antibacterial activity in one or more bodily excretions of the subject is eliminated.
   93. The composition of any one of Aspects 86 to 89 for controlling the dosing of antibacterial treatment of target bacteria in a human or animal subject, eg, in the gut of the subject.
   94. The composition of any one of Aspects 86 to 89 for fixing the dosing of antibacterial treatment of target bacteria in a human or animal subject, eg, in the gut of the subject.
   95. An isolated DNA comprising all structural protein genes of a helper phage genome that are required for producing phage particles, wherein the DNA is devoid of a helper phage production factor (HPF) that is required for producing packaged helper phage, optionally wherein the DNA comprises one or more promoters for expression of the genes when the DNA is integrated in the genome of a host bacterial cell.
   96. The DNA of Aspect 95, wherein the DNA is devoid of any phage packaging signals.
   97. The DNA of Aspect 95 or 96, wherein the HPF is a sigma factor-encoding nucleotide sequence or comprises a sigma factor recognition site, a DNA polymerisation recognition site, a promoter of a gene required for helper phage DNA replication, a helper phage integrase-encoding nucleotide sequence, a helper phage excissionase-encoding nucleotide sequence or a helper phage origin of replication.
   98. The DNA of any one of Aspects 95 to 97, wherein the DNA comprises a nucleotide sequence encoding a CRISPR/Cas system repressor.
   99. The DNA of any one of Aspects 95 to 98, wherein the DNA is integrated in the chromosome of a host bacterial cell, wherein the genes are expressible in the host cell.
   100. The DNA of Aspect 99, wherein the cell is devoid of an active CRISPR/Cas system.
   101. The DNA of any one of Aspects 95 to 100 in combination with a second DNA, wherein the second DNA comprises the HPF.
   102. The DNA of any one of Aspects 95 to 100 in combination with a second DNA, wherein the second DNA comprises a phage packaging signal and optionally the first DNA is devoid of a phage packaging signal.
   103. The DNA of Aspect 101 or 102, wherein the second DNA is comprised by a phagemid or a plasmid (eg, a shuttle vector).

In an example, the kit, DNA(s), first phage, helper phage or composition is comprised by a medical container, eg, a syringe, vial, IV bag, inhaler, eye dropper or nebulizer. In an example, the kit, DNA(s), first phage, helper phage or composition is comprised by a sterile container. In an example, the kit, DNA(s), first phage, helper phage or composition is comprised by a medically-compatible container. In an example, the kit, DNA(s), first first phage, helper phage or composition is comprised by a fermentation vessel, eg, a metal, glass or plastic vessel.

In an example, the kit, DNA(s), first phage, helper phage or composition is comprised by a medicament, e.g in combination with instructions or a packaging label with directions to administer the medicament by oral, IV, subcutaneous, intranasal, intraocular, vaginal, topical, rectal or inhaled administration to a human or animal subject. In an example, the kit, DNA(s), first phage, helper phage or composition is comprised by an oral medicament formulation. In an example, the kit, DNA(s), first phage, helper phage or composition is comprised by an intranasal or ocular medicament formulation. In an example, the kit, DNA(s), first phage, helper phage or composition is comprised by a personal hygiene composition (eg, shampoo, soap or deodorant) or cosmetic formulation. In an example, the kit, DNA(s), first phage, helper phage or composition is comprised by a detergent formulation. In an example, the kit, DNA(s), first phage, helper phage or composition is comprised by a cleaning formulation, eg, for cleaning a medical or industrial device or apparatus. In an example, the kit, DNA(s), first phage, helper phage or composition is comprised by foodstuff, foodstuff ingredient or foodstuff processing agent. In an example, the kit, DNA(s), first phage, helper phage or composition is comprised by beverage, beverage ingredient or beverage processing agent. In an example, the kit, DNA(s), first phage, helper phage or composition is comprised by a medical bandage, fabric, plaster or swab. In an example, the kit, DNA(s), first phage, helper phage or composition is comprised by a herbicide or pesticide. In an example, the kit, DNA(s), first phage, helper phage or composition is comprised by an insecticide.

In an example, the first phage is a is a Corticoviridae, Cystoviridae, Inoviridae, Leviviridae, Microviridae, Myoviridae, Podoviridae, Siphoviridae, or Tectiviridae virus. In an example, the helper phage is a is a Corticoviridae, Cystoviridae, Inoviridae, Leviviridae, Microviridae, Myoviridae, Podoviridae, Siphoviridae, or Tectiviridae virus. In an example, the helper phage is a filamentous M13, a Noviridae, a tailed phage (eg, a Myoviridae, Siphoviridae or Podoviridae), or a non-tailed phage (eg, a Tectiviridae).

In an example, both the first and helper phage are Corticoviridae. In an example, both the first and helper phage are Cystoviridae. In an example, both the first and helper phage are Inoviridae. In an example, both the first and helper phage are Leviviridae. In an example, both the first and helper phage are Microviridae. In an example, both the first and helper phage are Podoviridae. In an example, both the first and helper phage are Siphoviridae. In an example, both the first and helper phage are Tectiviridae.

In an example, the CRISPR/Cas component(s) are component(s) of a Type I CRISPR/Cas system. In an example, the CRISPR/Cas component(s) are component(s) of a Type II CRISPR/Cas system. In an example, the CRISPR/Cas component(s) are component(s) of a Type III CRISPR/Cas system. In an example, the CRISPR/Cas component(s) are component(s) of a Type IV CRISPR/Cas system. In an example, the CRISPR/Cas component(s) are component(s) of a Type V CRISPR/Cas system. In an example, the CRISPR/Cas component(s) comprise a Cas9-encoding nucleotide sequence (eg, S pyogenes Cas9, S aureus Cas9 or S thermophilus Cas9). In an example, the CRISPR/Cas component(s) comprise a Cas3-encoding nucleotide sequence (eg, E coli Cas3, C dificile Cas3 or Salmonella Cas3). In an example, the CRISPR/Cas component(s) comprise a Cpf-encoding nucleotide sequence. In an example, the CRISPR/Cas component(s) comprise a CasX-encoding nucleotide sequence. In an example, the CRISPR/Cas component(s) comprise a CasY-encoding nucleotide sequence.

In an example, the first DNA, first phage or vector encode a CRISPR/Cas component or protein of interest from a nucleotide sequence comprising a promoter that is operable in the target bacteria.

In an example, the host bacteria and/or target bacteria are E coli. In an example, the host bacteria and/or target bacteria are C dificile (eg, the vector is a shuttle vector operable in E coli and the host bacteria are C dificile). In an example, the host bacteria and/or target bacteria are Streptococcus, such as S thermophilus (eg, the vector is a shuttle vector operable in E coli and the host bacteria are Streptococcus). In an example, the host bacteria and/or target bacteria are Pseudomonas, such as P aeruginosa (eg, the vector is a shuttle vector operable in E coli and the host bacteria are P aeruginosa). In an example, the host bacteria and/or target bacteria are Klebsiella (eg, the vector is a shuttle vector operable in E coli and the host bacteria are Klebsiella). In an example, the host bacteria and/or target bacteria are Salmonella, eg, S typhimurium (eg, the vector is a shuttle vector operable in E coli and the host bacteria are Salmonella).

Optionally, host and/or target bacteria is a gram negative bacterium (eg, a spirilla or Vibrio). Optionally, host and/or target bacteria is a gram positive bacterium. Optionally, host and/or target bacteria is a mycoplasma, chlamydiae, spirochete or Mycobacterium. Optionally, host and/or target bacteria is a Streptococcus (eg, pyogenes or thermophilus). Optionally, host and/or target bacteria is a Staphylococcus (eg, aureus, eg, MRSA). Optionally, host and/or target bacteria is an E. coli (eg, 0157: H7) host, eg, wherein the Cas is encoded by the vector or an endogenous host Cas nuclease activity is de-repressed. Optionally, host and/or target bacteria is a Pseudomonas (eg, aeruginosa). Optionally, host and/or target bacteria is a Vibro (eg, cholerae (eg, 0139) or vulnificus). Optionally, host and/or target bacteria is a Neisseria (eg, gonnorrhoeae or meningitidis). Optionally, host and/or target bacteria is a Bordetella (eg, pertussis). Optionally, host and/or target bacteria is a Haemophilus (eg, influenzae). Optionally, host and/or target bacteria is a Shigella (eg, dysenteriae). Optionally, host and/or target bacteria is a Brucella (eg, abortus). Optionally, host and/or target bacteria is a Francisella host. Optionally, host and/or target bacteria is a Xanthomonas host. Optionally, host and/or target bacteria is a Agrobacterium host. Optionally, host and/or target bacteria is a Erwinia host. Optionally, host and/or target bacteria is a Legionella (eg, pneumophila). Optionally, host and/or target bacteria is a Listeria (eg, monocytogenes). Optionally, host and/or target bacteria is a Campylobacter (eg, jejuni). Optionally, host and/or target bacteria is a Yersinia (eg, pestis). Optionally, host and/or target bacteria is a Borelia (eg, burgdorferi). Optionally, host and/or target bacteria is a Helicobacter (eg, pylori). Optionally, host and/or target bacteria is a Clostridium (eg, dificile or botulinum). Optionally, host and/or target bacteria is a Erlichia (eg, chaffeensis). Optionally, host and/or target bacteria is a Salmonella (eg, typhi or enterica, eg, serotype typhimurium, eg, DT 104). Optionally, host and/or target bacteria is a Chlamydia (eg, pneumoniae). Optionally, host and/or target bacteria is a Parachlamydia host. Optionally, host and/or target bacteria is a Corynebacterium (eg, amycolatum). Optionally, host and/or target bacteria is a Klebsiella (eg, pneumoniae). Optionally, host and/or target bacteria is an Enterococcus (eg, faecalis or faecim, eg, linezolid-resistant). Optionally, host and/or target bacteria is an Acinetobacter (eg, baumannii, eg, multiple drug resistant).

Further examples of target cells and targeting of antibiotic resistance in such cells using the present invention are as follows:—
1. Optionally the target bacteria are Staphylococcus aureus cells, eg, resistant to an antibiotic selected from methicillin, vancomycin, linezolid, daptomycin, quinupristin, dalfopristin and teicoplanin.
2. Optionally the target bacteria are Pseudomonas aeuroginosa cells, eg, resistant to an antibiotic selected from cephalosporins (eg, ceftazidime), carbapenems (eg, imipenem or meropenem), fluoroquinolones, aminoglycosides (eg, gentamicin or tobramycin) and colistin.
3. Optionally the target bacteria are Klebsiella (eg, pneumoniae) cells, eg, resistant to carbapenem.
4. Optionally the target bacteria are Streptoccocus (eg, thermophilus, pneumoniae or pyogenes) cells, eg, resistant to an antibiotic selected from erythromycin, clindamycin, beta-lactam, macrolide, amoxicillin, azithromycin and penicillin.
5. Optionally the target bacteria are Salmonella (eg, serotype Typhi) cells, eg, resistant to an antibiotic selected from ceftriaxone, azithromycin and ciprofloxacin.
6. Optionally the target bacteria are Shigella cells, eg, resistant to an antibiotic selected from ciprofloxacin and azithromycin.
7. Optionally the target bacteria are Mycobacterium tuberculosis cells, eg, resistant to an antibiotic selected from Resistance to isoniazid (INH), rifampicin (RMP), fluoroquinolone, amikacin, kanamycin and capreomycin and azithromycin.
8. Optionally the target bacteria are Enterococcus cells, eg, resistant to vancomycin.
9. Optionally the target bacteria are Enterobacteriaceae cells, eg, resistant to an antibiotic selected from a cephalosporin and carbapenem.
10. Optionally the target bacteria are E. coli cells, eg, resistant to an antibiotic selected from trimethoprim, itrofurantoin, cefalexin and amoxicillin
11. Optionally the target bacteria are Clostridium (eg, dificile) cells, eg, resistant to an antibiotic selected from fluoroquinolone antibiotic and carbapenem.
12. Optionally the target bacteria are Neisseria gonnorrhoea cells, eg, resistant to an antibiotic selected from cefixime (eg, an oral cephalosporin), ceftriaxone (an injectable cephalosporin), azithromycin and tetracycline.
13. Optionally the target bacteria are Acinetoebacter baumannii cells, eg, resistant to an antibiotic selected from beta-lactam, meropenem and a carbapenem.
14. Optionally the target bacteria are Campylobacter cells, eg, resistant to an antibiotic selected from ciprofloxacin and azithromycin.
15. Optionally, the target cell(s) produce Beta (β)-lactamase.
16. Optionally, the target cell(s) are bacterial cells that are resistant to an antibiotic recited in any one of examples 1 to 14.

### Mobile Genetic Elements, Genomic Islands, Pathogenicity Islands Etc.

Genetic variation of bacteria and archaea can be achieved through mutations, rearrangements and horizontal gene transfers and recombinations. Increasing genome sequence data have demonstrated that, besides the core genes encoding house-keeping functions such as essential metabolic activities, information processing, and bacterial structural and regulatory components, a vast number of accessory genes encoding antimicrobial resistance, toxins, and enzymes that contribute to adaptation and survival under certain environmental conditions are acquired by horizontal gene transfer of mobile genetic elements (MGEs). Mobile genetic elements are a heterogeneous group of molecules that include plasmids, bacteriophages, genomic islands, chromosomal cassettes, pathogenicity islands, and integrative and conjugative elements. Genomic islands are relatively large segments of DNA ranging from 10 to 200 kb often integrated into tRNA gene clusters flanked by 16-20 bp direct repeats. They are recognized as discrete DNA segments acquired by horizontal gene transfer since they can differ from the rest of the chromosome in terms of GC content (% G+C) and codon usage.

Pathogenicity islands (PTIs) are a subset of horizontally transferred genetic elements known as genomic islands. There exists a particular family of highly mobile PTIs in Staphylococcus aureus that are induced to excise and replicate by certain resident prophages. These PTIs are packaged into small headed phage-like particles and are transferred at frequencies commensurate with the plaque-forming titer of the phage. This process is referred to as the SaPI excision replication-packaging (ERP) cycle, and the high-frequency SaPI transfer is referred to as SaPI-specific transfer (SPST) to distinguish it from classical generalized transduction (CGT). The SaPIs have a highly conserved genetic organization that parallels that of bacteriophages and clearly distinguishes them from all other horizontally acquired genomic islands. The SaPThencoded and SaPIbov2-encoded integrases are used for both excision and integration of the corresponding elements, and it is assumed that the same is true for the other SaPIs. Phage 80α can induce several different SaPIs, including SaPI1, SaPI2, and SaPIbov1, whereas φ11 can induce SaPIbov1 but neither of the other two SaPIs.

Reference is made to “Staphylococcal pathogenicity island DNA packaging system involving cos-site packaging and phage-encoded HNH endonucleases”, Quiles-Puchalt et al, PNAS Apr. 22, 2014. 111 (16) 6016-6021. Staphylococcal pathogenicity islands (SaPIs) are highly mobile and carry and disseminate superantigen and other virulence genes. It was reported that SaPIs hijack the packaging machinery of the phages they victimise, using two unrelated and complementary mechanisms. Phage packaging starts with the recognition in the phage DNA of a specific sequence, termed “pac” or “cos” depending on the phage type. The SaPI strategies involve carriage of the helper phage pac- or cos-like sequences in the SaPI genome, which ensures SaPI packaging in full-sized phage particles, depending on the helper phage machinery. These strategies interfere with phage reproduction, which ultimately is a critical advantage for the bacterial population by reducing the number of phage particles.

Staphylococcal pathogenicity islands (SaPIs) are the prototypical members of a widespread family of chromosomally located mobile genetic elements that contribute substantially to intra- and interspecies gene transfer, host adaptation, and virulence. The key feature of their mobility is the induction of SaPI excision and replication by certain helper phages and their efficient encapsidation into phage-like infectious particles. Most SaPIs use the headful packaging mechanism and encode small terminase subunit (TerS) homologs that recognize the SaPI-specific pac site and determine SaPI packaging specificity. Several of the known SaPIs do not encode a recognizable TerS homolog but are nevertheless packaged efficiently by helper phages and transferred at high frequencies. Quiles-Puchalt et al report that one of the non-terS-coding SaPIs, SaPIbov5, and found that it uses two different, undescribed packaging strategies. SaPIbov5 is packaged in full-sized phage-like particles either by typical pac-type helper phages, or by cos-type phages—i.e., it has both pac and cossites and uses the two different phage-coded TerSs. This is an example of SaPI packaging by a cos phage, and in this, it resembles the P4 plasmid of Escherichia coli. Cos-site packaging in Staphylococcus aureus is additionally unique in that it requires the HNH nuclease, carried only by cos phages, in addition to the large terminase subunit, for cos-site cleavage and melting.

Characterization of several of the phage-inducible SaPIs and their helper phages has established that the pac (or headful) mechanism is used for encapsidation. In keeping with this concept, some SaPIs encode a homolog of TerS, which complexes with the phage-coded large terminase subunit TerL to enable packaging of the SaPI DNA in infectious particles composed of phage proteins. These also contain a morphogenesis (cpm) module that causes the formation of small capsids commensurate with the small SaPI genomes. Among the SaPI sequences first characterized, there were several that did not include either a TerS homolog or a cpm homolog, and the same is true of several subsequently identified SaPIs from bovine sources and for many phage-inducible chromosomal islands from other species. It was assumed, for these several islands, either that they were defective derivatives of elements that originally possessed these genes, or that terS and cpm genes were present but not recognized by homology.

Quiles-Puchalt et al observed that an important feature of ϕSLT/SaPIbov5 packaging is the requirement for an HNH nuclease, which is encoded next to the ϕSLT terminase module. Proteins carrying HNH domains are widespread in nature, being present in organisms of all kingdoms. The HNH motif is a degenerate small nucleic acid-binding and cleavage module of about 30-40 aa residues and is bound by a single divalent metal ion. The HNH motif has been found in a variety of enzymes playing important roles in many different cellular processes, including bacterial killing; DNA repair, replication, and recombination; and processes related to RNA. HNH endonucleases are present in a number of cos-site bacteriophages of Gram-positive and -negative bacteria, always adjacent to the genes encoding the terminases and other morphogenetic proteins. Quiles-Puchalt et al have demonstrated that the HNH nucleases encoded by ϕ12 and the closely related ϕSLT have nonspecific nuclease activity and are required for the packaging of these phages and of SaPIbov5. Quiles-Puchalt et al have shown that HNH and TerL are jointly required for cos-site cleavage. Quiles-Puchalt et al have also observed that only cos phages of Gram-negative as well as of Gram-positive bacteria encode HNH nucleases, consistent with a special requirement for cos-site cleavage as opposed to pac-site cleavage, which generates flush-ended products. The demonstration that HNH nuclease activity is required for some but not other cos phages suggests that there is a difference between the TerL proteins of the two types of phages—one able to cut both strands and the other needing a second protein to enable the generation of a double-stranded cut.

The invention, also involves, in certain configurations the use of mobile genetic elements (MGEs). Thus, there are provided the following Clauses. Any of the other configurations, Aspects, Examples or description of the invention above or elsewhere herein are combinable mutatis mutandis with any of these Clauses:—
1. A composition for use in antibacterial treatment of bacteria, the composition comprising an engineered mobile genetic element (MGE) that is capable of being mobilised in a first bacterial host cell of a first species or strain, the cell comprising a first phage genome, wherein in the cell the MGE is mobilised using proteins encoded by the phage and replication of first is inhibited, wherein the MGE encodes an antibacterial agent or encodes a component of such an agent.
   In the alternative, instead of a bacteria, the host cell is a archaeal cell and instead of a phage there is a virus that is capable of infecting the archaeal cell.
   In an example, the MGE is capable of integration into the genome of the host cell comprising the genome of a first phage, for example integration in the chromosome of the host cell and/or an episome thereof.
   Optionally, the MGE inhibits first phage replication.
   In an example, first phage replication is totally inhibited. In an example, it is reduced by at least 50, 60, 70, 80 or 90% compared to replication in the absence of the MGE in host cells. This can be assessed by a standard in vitro plaque assay to determine the relative amount of first phage plaque formation.
   Optionally, in the presence of the agent,
   (i) host cells are killed by the antibacterial agent;
   (ii) growth or proliferation of host cells is reduced; and/or
   (iii) host cells are sensitised to an antibiotic, whereby the antibiotic is toxic to the cells.
2. The composition of Clause 1, wherein the agent is toxic to cells of the same species or strain as the host cell.
3. The composition of Clause 1 or 2, wherein the agent is toxic to cells of a species or strain that is different from the strain or species of the host cell.
4. The composition of Clause 1, wherein the agent is toxic to cells of the same species as the host cell, and wherein the host cell has been engineered so that the agent is not toxic to the host cell.
5. The composition of Clause 4, wherein the agent is a guided nuclease system (optionally a CRISPR/Cas system) and cells of the same species as the host cell comprise a target sequence that is cut by the nuclease, wherein the target sequence has been removed or altered in the host cell whereby the nuclease is not capable of cutting the target sequence.
   Viruses undergo lysogenic and lytic cycles in a host cell. If the lysogenic cycle is adopted, the phage chromosome can be integrated into the bacterial chromosome, or it can establish itself as a stable plasmid in the host, where it can remain dormant for long periods of time. If the lysogen is induced, the phage genome is excised from the bacterial chromosome and initiates the lytic cycle, which culminates in lysis of the cell and the release of phage particles. The lytic cycle leads to the production of new phage particles which are released by lysis of the host.
6. The composition of any preceding Clause, wherein the first phage is a temperate phage.
7. The composition of any preceding Clause, wherein the first cell comprises the first phage as a prophage.
8. The composition of any one of Clauses 1 to 5, wherein the first phage is a lytic phage.
9. The composition of any preceding Clause, wherein in the presence of a first phage the mobilisation of the MGE causes host cell lysis.
10. The composition of any preceding Clause, wherein the MGE is capable of being packaged in transduction particles that comprise some, but not all, structural proteins of the first phage.
   “Transduction particles” may be phage or smaller than phage and are particles that are capable of transducing nucleic acid encoding the antibiotic or component thereof into target bacterial cells.
   Examples of structural proteins are phage proteins selected from one, more or all of the major head and tail proteins, the portal protein, tail fibre proteins, and minor tail proteins.
   The MGE comprises a packaging signal sequence operable with proteins encoded by the first phage to package the MGE (or at least nucleic acid thereof encoding the agent or one or more components thereof) into transduction particles that are capable of infecting host cells of the same species or strain as the first host cell.
11. The composition of any preceding Clause, wherein mobilisation of the MGE comprises packaging of copies of the MGE or nucleic acid encoding the agent or component into transduction particles that are capable of transferring the copies into target bacterial cells for antibacterial treatment of the target cells.
12. The composition of Clause 10 or 11, wherein the transduction particles are particles of second phage that are capable of infecting cells of said first species or strain.
13. The composition of any one of Clauses 10 to 12, wherein the transduction particles are non-self replicative particles.

A “non-self replicative transduction particle” refers to a particle, (eg, a phage or phage-like particle; or a particle produced from a genomic island (eg, a SaPI) or a modified version thereof) capable of delivering a nucleic acid molecule encoding an antibacterial agent or component into a bacterial cell, but does not package its own replicated genome into the transduction particle. In an alternative herein, instead of a phage, there is used or packaged a virus that infects an animal, human, plant or yeast cell. For example, an adenovirus when the cell is a human cell.
14. The composition of any preceding Clause, wherein the MGE is devoid of genes encoding phage structural proteins.
   Optionally, the MGE is devoid of one or more phage genes rinA, terS and terL.
   In an example, in a host cell a protein complex comprising the small terminase (encoded by terS) and large terminase (encoded by terL) proteins is able to recognise and cleave a double-stranded DNA molecule of the MGE at or near the pac site (cos site or other packaging signal sequence comprised by the MGE), and this allows the MGE or plasmid DNA molecule to be packaged into a phage capsid. When first phage as prophage in the host cell is induced, the lytic cycle of the phage produces the phage's structural proteins and the phage's large terminase protein. The MGE or plasmid is replicated, and the small terminase protein encoded by the MGE or plasmid is expressed. The replicated MGE or plasmid DNA containing the terS (and the nucleotide sequence encoding the antibacterial agent or component) are packaged into phage capsids, resulting in non-self replicative transduction particles carrying only MGE or plasmid DNA.
15. The composition of any one of Clauses 1 to 13, wherein the MGE comprises phage structural genes and a packaging signal sequence and the first phage is devoid of a packaging signal sequence.
16. The composition of any preceding Clause, wherein the MGE is a modified version of a MGE that is naturally found in bacterial cells of the first species or strain.
17. The composition of any preceding Clause, wherein the MGE comprises a modified genomic island.
   Optionally, the genomic island is an island that is naturally found in bacterial cells of the first species or strain. In an example, the genomic island is selected from the group consisting of a SaPI, a SaPI1, a SaPI2, a SaPIbov1 and a SaPibov2 genomic island.
18. The composition of any preceding Clause, wherein the MGE comprises a modified pathogenicity island.
   Optionally, the pathogenicity island is an island that is naturally found in bacterial cells of the first species or strain, eg, a Staphylococcus SaPI or a Vibro PLE or a P. aeruginosa pathogenicity island (eg, a PAPI or a PAGI, eg, PAPI-1, PAGI-5, PAGI-6, PAGI-7, PAGI-8, PAGI-9, PAGI-10, or PAGI—
19. The composition of Clause 18, wherein the pathogenicity island is a SaPI (S aureus pathogenicity island).
20. The composition of Clause 19, wherein the first phage is ϕ11, 80α, ϕ12 or ϕSLT. Staphylococcus phage 80α appears to mobilise all known SaPIs. Thus, in an example, the MGE comprises a modified SaPI and the first phage is a 80α.
21. The composition of Clause 18, wherein the pathogenicity island is a V. cholerae PLE (phage-inducible chromosomal island-like element) and optionally the first phage is ICP1.
22. The composition of Clause 18, wherein the pathogenicity island is a E coli PLE.
23. The composition of any one of Clauses 1 to 16, wherein the MGE comprises P4 DNA, eg, a P4 packaging signal sequence.
24. The composition of Clause 23, wherein the first phage are P2 phage or a modified P2 phage that is self-replicative defective; optionally present as a prophage.
25. The composition of any preceding Clause, wherein the MGE comprises a pacA gene of the Enterobacteriaceae bacteriophage P1.
26. The composition of any preceding Clause, wherein the MGE comprises a packaging initiation site sequence, optionally a packaging initiation site sequence of P1.
27. The composition of any preceding Clause, wherein the MGE comprises a nucleotide sequence that is beneficial to cells of the first species or strain, optionally encoding a protein that is beneficial to cells of the first species or strain.
   This is useful where, not only does the presence of the MGE reduce first phage replication in the host cell, but also the MGE is taken up and may provide a survival, growth or other benefit to the host cell, promoting uptake and/or retention of MGEs by host cells. In an example, expression of the antibacterial agent in the host cell is under the control of an inducible promoter or weak promoter to allow for a period where uptake of MGEs into host cells may be favoured owing to the presence of the nucleotide sequence that is beneficial to cells of the first species or strain.
28. The composition of any preceding Clause, wherein the MGE is devoid of rinA.
29. The composition of any preceding Clause, wherein the MGE is is devoid of terL.
30. The composition of any preceding Clause, wherein the MGE comprises a terS or a homologue thereof, and optionally is devoid of any other terminase gene.
   The terS homologues are sequences which, like terS, recognise the SaPI-specific pac site (or other packaging sequence) comprised by the MGE or plasmid and determine packaging specificity for packaging the MGE.
   Examples of terminase genes are pacA, pacB, terA, terB and terL.
31. The composition of any preceding Clause, wherein the first phage is a pac-type phage (eg, ϕ11 or 80α) operable with a pac comprised by the MGE.
32. The composition of any one of Clauses 1 to 30, wherein the first phage is a cos-type phage (eg, ϕ12 or ϕSLT) operable with a cos comprised by the MGE.
   Optionally, the phage is P2. Optionally, the first phage is a T7 or T7-like phage that recognises direct repeat sequences comprised by the MGE for packaging.
33. The composition of any preceding Clause, wherein the plasmid or MGE comprises a pac and/or cos sequence or a homologue thereof.
34. The composition of any preceding Clause, wherein the plasmid or MGE comprises a terS or a homologue thereof and optionally devoid of terL.
   The terS homologues are sequences which, like terS, recognise the SaPI-specific pac site (or other packaging sequence) comprised by the MGE or plasmid and determine packaging specificity for packaging the MGE.
   In an example, the terS comprises the sequence of SEQ ID NO: 2:—

SEQ ID NO: 2
AATTGGCAGTAAAGTGGCAGTTTTGATACCTAAAATGAGATATTATGATA
GTGTAGGATATTGACTATCTTACTGCGTTTCCCTTATCGCAATTAGGAAT
AAAGGATCTATGTGGGTTGGCTGATTATAGCCAATCCTTTTTTAATTTTA
AAAAGCGTATAGCGCGAGAGTTGGTGGTAAATGAAATGAACGAAAAACAA
AAGAGATTCGCAGATGAATATATAATGAATGGATGTAATGGTAAAAAAGC
AGCAATTTCAGCAGGTTATAGTAAGAAAACAGCAGAGTCTTTAGCAAGTC
GATTGTTAAGAAATGTTAATGTTTCGGAATATATTAAAGAACGATTAGAA
CAGATACAAGAAGAGCGTTTAATGAGCATTACAGAAGCTTTAGCGTTATC
TGCTTCTATTGCTAGAGGAGAACCTCAAGAGGCTTACAGTAAGAAATATG
ACCATTTAAACGATGAAGTGGAAAAAGAGGTTACTTACACAATCACACCA
ACTTTTGAAGAGCGTCAGAGATCTATTGACCACATACTAAAAGTTCATGG
TGCGTATATCGACAAAAAAGAAATTACTCAGAAGAATATTGAGATTAATA
TTGGTGAGTACGATGACGAAAGTTAAATTAAACTTTAACAAACCATCTAA
TGTTTTCAACAG

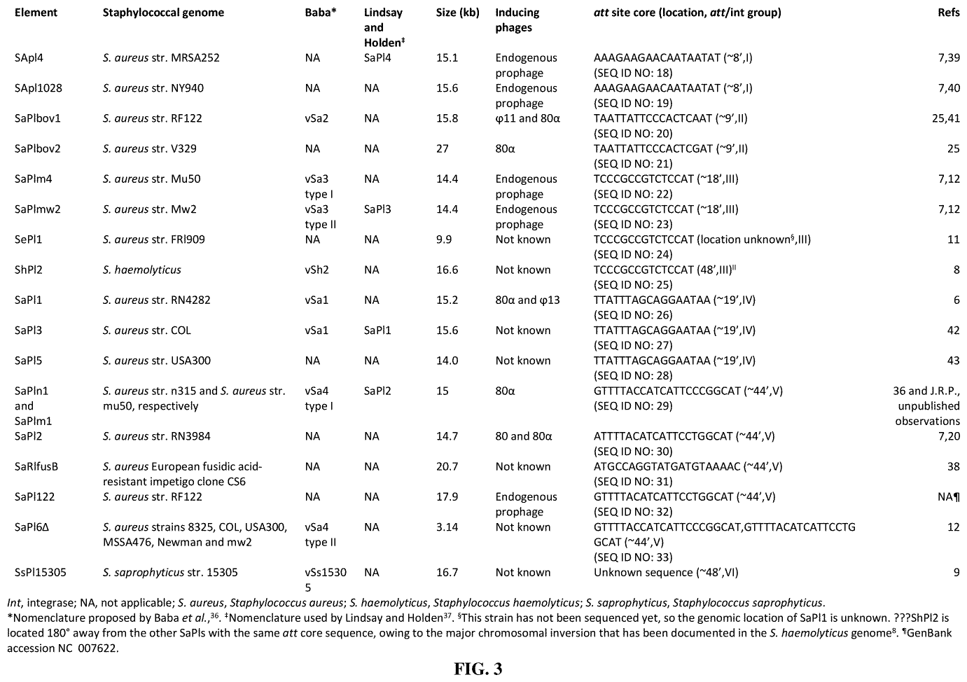

35. The composition of Clause 34, wherein the terS is a S aureus bacteriophage φ80α terS or a bacteriophage φ11 terS.
36. The composition of any preceding Clause, wherein the MGE is a modified SaPIbov1 or SaPIbov5 and is devoid of a terS.
37. The composition of any preceding Clause, wherein the first phage is devoid of a functional packaging signal sequence and the MGE comprises a packaging signal sequence operable with proteins encoded by the first phage for producing transduction particles that package copies of the MGE or copies of a nucleic acid encoding the agent or component.
38. The composition of any preceding Clause, wherein the MGE or plasmid comprises a Ppi or homologue, which is capable of complexing with first phage TerS, thereby blocking function of the TerS.
39. The composition of any preceding Clause, wherein the MGE comprises a morphogenesis (cpm) module.
40. The composition of any preceding Clause, wherein the MGE comprises cpmA and/or cpmB.
   Optionally the cpmA and B are from any SaPI disclosed herein. In an example any SaPI is a SaPI disclosed in FIG. 3 and optionally the host cell or target cell is any corresponding Staphylococcus disclosed in the table.
41. The composition of any preceding Clause, wherein the MGE or first phage comprises one, more or all genes cp1, cp2, and cp3.
   In an example, the MGE comprises a modified SaPI and comprises one, more or all genes cp1, cp2, and cp3.
42. The composition of any preceding Clause, wherein the MGE or first phage encodes a HNH nuclease.
43. The composition of any preceding Clause, wherein the MGE or first phage comprises an integrase gene that encodes an integrase for excising the MGE and integrating the MGE into a bacterial cell genome.
44. The composition of any preceding Clause, wherein the MGE is devoid of a functional integrase gene, and the first phage or host cell genome (eg, bacterial chromosome or a bacterial episome) comprises a functional integrase gene.
45. The composition of any preceding Clause, wherein the transcription of MGE nucleic acid is under the control of a constitutive promoter, for transcription of copies of the agent or component in a host cell.
   Optionally, Constitutive transcription and production of the agent in target cells may be used where the target cells should be killed, eg, in medical settings.
   Optionally, the transcription of MGE nucleic acid is under the control of an inducible promoter, for transcription of copies of the agent or component in a host cell. This may be useful, for example, to control switching on of the antibacterial activity against target bacterial cells, such as in an environment (eg, soil or water) or in an industrial culture or fermentation container containing the target cells. For example, the target cells may be useful in an industrial process (eg, for fermentation, eg, in the brewing or dairy industry) and the induction enables the process to be controlled (eg, stopped or reduced) by using the antibacterial agent against the target bacteria.
46. The composition of Clause 45, wherein the promoter is foreign to the host cell.
47. The composition of Clause 45 or 46, wherein the promoter comprises a nucleotide sequence that is at least 80% identical to an endogenous promoter sequence of the host cell.
48. The composition of any preceding Clause comprising a nucleic acid that is separate from the MGE, wherein the nucleic acid comprises all genes necessary for producing first phage particles.
49. The composition of any one of Clauses 1 to 47 comprising a nucleic acid that is separate from the MGE, wherein the nucleic acid comprises less than, all genes necessary for producing first phage particles, but comprises genes encoding structural proteins for production of transduction particles that package MGE nucleic acid encoding the antibacterial agent or one or more components thereof.
   When the agent comprises a plurality of components, eg, wherein the agent is a CRISPR/Cas system, or is a CRISPR array encoding crRNA or a nucleic acid encoding a guide RNA (eg, single guide RNA) operable with a Cas in host cells, wherein the crRNA or gRNA guides the Cas to a target sequence in the host cell to modify the target (eg, cut it or repress transcription from it).
50. The composition of Clause 48 or 49, wherein the genes are comprised by the host cell chromosome and/or one or more host cell episome(s).
51. The composition of Clause 50, wherein the genes are comprised by a chromosomally-integrated prophage of the first phage.
52. The composition of any preceding Clause, wherein the agent is a guided nuclease system or a component thereof, wherein the agent is capable of recognising and cutting host cell DNA (eg, chromosomal DNA).
   In examples, such cutting causes one or more of the following:—
   (i) The host cel is killed by the antibacterial agent;
   (ii) growth or proliferation of the host cell is reduced; and/or
   (iii) The host cell is sensitised to an antibiotic, whereby the antibiotic is toxic to the cell.
53. The composition of Clause 52, wherein the guided nuclease system is selected from a CRISPR/Cas system, TALEN system, meganuclease system or zinc finger system.
54. The composition of Clause 52, wherein the system is a CRISPR/Cas system and each MGE encodes a (a) CRISPR array encoding crRNA or (b) a nucleic acid encoding a guide RNA (gRNA, eg, single guide RNA), wherein the crRNA or gRNA is operable with a Cas in target bacterial cells, wherein the crRNA or gRNA guides the Cas to a target nucleic acid sequence in the host cell to modify the target sequence (eg, cut it or repress transcription from it).
   Optionally, the Cas is a Cas encoded by a functional endogenous nucleic acid of a host cell. For example, the target is comprised by a DNA or RNA of the host cell.
55. The composition of Clause 52, wherein the system is a CRISPR/Cas system and each MGE encodes a Cas (eg, a Cas nuclease) that is operable in a target bacterial cells to modify a target nucleic acid sequence comprised by the target cell.
56. The composition of Clause 53, 54 or 55, wherein the Cas is a Cas3, Cas9, Cas13, CasX, CasY or Cpf1.
57. The composition of any one of Clauses 52 to 56, wherein the system is a CRISPR/Cas system and each MGE encodes one or more Cascade Cas (eg, Cas, A, B, C, D and E).
58. The composition of any one of Clauses 52 to 57, wherein each MGE further encodes a Cas3 that is operable in a target bacterial cell with the Cascade Cas.
59. The composition of any preceding Clause, wherein the first species or strain is a gram positive species or strain.
60. The composition of any one of Clauses 1 to 58, wherein the first species or strain is a gram negative species or strain.
61. The composition of any preceding Clause, wherein the first species or strain is selected from Table 1.
   In an example, the first species of strain is a Staphylococcus (eg, S aureus) species or strain and optionally the MGE is a modified SaPI; and optionally the first phage is a φ80α or φ11. In an example, the first species of strain is a Vibrio (eg, V cholerae) species or strain and optionally the MGE is Vibrio (eg, V cholerae) PLE.
62. The composition of any preceding Clause, wherein the first species or strain is selected from Shigella, E coli, Salmonella, Serratia, Klebsiella, Yersinia, Pseudomonas and Enterobacter.
   These are species that P2 phage can infect. Thus, in an embodiment, the MGE comprises one or more P4 sequences (eg, a P4 packaging sequence) and the first phage is P2. Thus, the MGE is packaged by P2 structural proteins and the resultant transduction particles can infect a broad spectrum of species, ie, two or more of Shigella, E coli, Salmonella, Serratia, Klebsiella, Yersinia, Pseudomonas and Enterobacter.
63. A nucleic acid vector comprising a MGE integrated therein, wherein the MGE is according to any preceding Clause and the vector is capable of transferring the MGE or a copy thereof into a host bacterial cell.
   Suitable vectors are plasmids (eg, conjugative plasmids) or viruses (eg, phage or packaged phagemids).
64. The vector of Clause 63, wherein the vector is a shuttle vector.
   A shuttle vector is a vector (usually a plasmid) constructed so that it can propagate in two different host species. Therefore, DNA inserted into a shuttle vector can be tested or manipulated in two different cell types.
65. The vector of Clause 63, wherein the vector is a plasmid, wherein the plasmid is capable of being transformed into a host bacterial cell comprising a first phage.
66. A non-self replicative transduction particle comprising said MGE or vector of any preceding Clause.
   By “non-replicative” it is meant that the MGE is not capable by itself of self-replicating. For example, the MGE is devoid of one or more nucleotide sequences encoding a protein (eg, a structural protein) that is necessary to produce a transduction particle comprising a copy of the MGE.
67. A composition comprising a plurality of transduction particles, wherein each particle comprises a MGE or vector according to any one of Clauses 1 to 65, wherein the transduction particles are capable of transferring the MGEs, or nucleic acid encoding the agent or component, or copies thereof into target bacterial cells, wherein
   a. target cells are killed by the antibacterial agent;
   b. growth or proliferation of target cells is reduced; or
   c. target cells are sensitised to an antibiotic, whereby the antibiotic is toxic to the cells.
      In an example, the reduction in growth or proliferation of host cells is at least 50, 60, 70, 80, 90 or 95%. The antibiotic can be any antibiotic disclosed herein.
68. The composition of Clause 67, wherein the agent is a guided nuclease system or a component thereof, wherein the agent is capable of recognising and cutting host cell DNA (eg, chromosomal DNA) whereby
   a. target cells are killed by the antibacterial agent;
   b. growth or proliferation of target cells is reduced; or
   c. target cells are sensitised to an antibiotic, whereby the antibiotic is toxic to the cells.
69. A composition comprising a plurality of non-self replicative transduction particles, wherein each particle comprises a MGE or plasmid according to any one of Clauses 1 to 65, wherein the transduction particles are capable of transferring the MGEs, or nucleic acid encoding the agent or component, or copies thereof into target bacterial cells, wherein the agent is a CRISPR/Cas system and the component comprises a nucleic acid encoding a crRNA or a guide RNA that is operable with a Cas in a target bacterial cell to guide the Cas to a target nucleic acid sequence of the cell to modify the sequence, whereby
   a. target cells are killed by the antibacterial agent;
   b. growth or proliferation of target cells is reduced; or
   c. target cells are sensitised to an antibiotic, whereby the antibiotic is toxic to the cells.
      In an example, the reduction in growth or proliferation of host cells is at least 50, 60, 70, 80, 90 or 95%. The antibiotic can be any antibiotic disclosed herein.
70. A kit comprising the composition of Clause 69 and said antibiotic.
71. The composition of Clause 69, wherein the composition comprises said antibiotic.
72. The composition of any one of Clauses 67 to 69, wherein less than 10% of transduction particles comprise by the composition are first phage particles.
73. The composition of any one of Clauses 67 to 69, wherein no first phage particles are present in the composition.
74. The MGE, vector, particle, composition or kit of any preceding Clause for medical use in a human or animal patient.
75. The MGE, vector, particle, composition or kit of any preceding Clause for treating or preventing an infection by target bacterial cells in a human or animal patient, wherein the antibacterial agent is toxic to the target cells.
76. The MGE, vector, particle, composition or kit of any preceding Clause for treating or preventing an infection by target bacterial cells in a human or animal patient, wherein in the presence of the antibacterial agent
   a. target cells are killed by the antibacterial agent;
   b. growth or proliferation of target cells is reduced; and/or
   c. target cells are sensitised to an antibiotic, whereby the antibiotic is toxic to the cells.
77. A method of producing a plurality of transduction particles, the method comprising combining the composition of any one of Clauses 1 to 62, 67 to 69 and 71 to 76 with host bacterial cells of said first species, wherein the cells comprise the first phage, allowing a plurality of said MGEs to be introduced into host cells and culturing the host cells under conditions in which first phage-encoded proteins are expressed and MGE copies are packaged by first phage proteins to produce a plurality of transduction particles, and optionally separating the transduction particles from cells and obtaining a plurality of transduction particles separated from cells.
78. The method of Clause 77, comprising separating the transduction particles from any first phage, optionally by filtering or centrifugation, thereby obtaining a plurality of transduction particles in the absence of first phage.
79. The method of Clause 77 or 78, wherein the particles encode a guided nuclease system (optionally a CRISPR/Cas system) or component thereof for cutting a target nucleic acid sequence comprised by target bacterial cells.
80. The method of Clause 79, wherein the sequence is comprised by an antibiotic resistance gene and the method comprises combining the plurality of particles with said antibiotic in a kit or a mixture.
81. The method of any one of Clauses 77 to 80, wherein said conditions comprise induction of a lytic cycle of the first phage.
82. A bacterial host cell comprising a first phage and a MGE, vector or particle as recited in any one of Clauses 1 to 66, wherein the agent is toxic to cells of the same species as the host cell, and wherein the host cell has been engineered so that the agent is not toxic to the host cell.
83. A bacterial host cell comprising a first phage, wherein the cell is comprised by a kit, the kit further comprising a composition as recited in any one of Clauses 1 to 62, 67 to 69 and 71 to 76, wherein the agent is toxic to cells of the same species as the host cell, and wherein the host cell has been engineered so that the agent is not toxic to the host cell.
84. The cell of Clause 83, wherein the agent is a guided nuclease system (optionally a CRISPR/Cas system) and cells of the same species as the host cell comprise a target sequence that is cut by the nuclease, wherein the target sequence has been removed or altered in the host cell whereby the nuclease is not capable of cutting the target sequence.
85. A bacterial host cell comprising a first phage and a MGE, vector or particle as recited in any one of Clauses 1 to 66, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.
86. A bacterial host cell comprising a first phage, wherein the cell is comprised by a kit, the kit further comprising a composition as recited in any one of Clauses 1 to 62, 67 to 69 and 71 to 76, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.
87. The cell of Clause 86, wherein the first phage is a prophage.
88. A bacterial host cell comprising a MGE, vector or particle as recited in any one of Clauses 1 to 66 and nucleic acid under the control of one or more inducible promoters, wherein the nucleic acid encodes all structural proteins necessary to produce a transduction particle that packages a copy of the MGE or plasmid, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.
89. The cell of Clause 88, wherein the structural proteins are structural proteins of a lytic phage.
90. The cell of Clause 88 or 89, wherein the nucleic acid comprises terS and/or terL.
91. The cell of any one of Clauses 88 to 90, wherein the host and second cells are of the same species and the host cell has been engineered so that the antibiotic is not toxic to the host cell.
92. The cell of any one of Clauses 88 to 91, wherein the nucleic acid is comprised by a plasmid.
93. The cell of any one of Clauses 88 to 92, wherein the agent is a guided nuclease system (optionally a CRISPR/Cas system) and the second cells comprise a target sequence that is cut by the nuclease, wherein the target sequence is absent in the genome of the host cell whereby the nuclease is not capable of cutting the host cell genome.
94. The composition, vector, particle, kit or method of any preceding Clause, wherein the cell, host cell or target cell is selected from a Staphylococcal, Vibrio, Pseudomonas, Clostridium, E coli, Helicobacter, Klebsiella and Salmonella cell.
95. A plasmid comprising
   a. A nucleotide sequence encoding an antibacterial agent or component thereof for expression in target bacterial cells;
   b. A constitutive promoter for controlling the expression of the agent or component;
   c. An optional terS nucleotide sequence;
   d. An origin of replication (ori); and
   e. A phage packaging sequence (optionally pac, cos or a homologue thereof); and
   f. the plasmid being devoid of
   g. All nucleotide sequences encoding phage structural proteins necessary for the production of a transduction particle (optionally a phage), or the plasmid being devoid of at least one of such sequences; and
   h. Optionally terL.
96. The plasmid of Clause 95, wherein the antibacterial agent is a CRISPR/Cas system and the plasmid encodes a crRNa or guide RNA (eg, single gRNA) that is operable with a Cas in the target cells to guide the Cas to a target nucleotide sequence to modify (eg, cut) the sequence, whereby
   a. target cells are killed by the antibacterial agent;
   b. growth or proliferation of target cells is reduced; or
   c. target cells are sensitised to an antibiotic, whereby the antibiotic is toxic to the cells.
97. The plasmid of Clause 95 or 96, wherein the antibacterial agent is a CRISPR/Cas system and the plasmid encodes a Cas that is operable with a crRNa or guide RNA (eg, single gRNA) in the target cells to guide the Cas to a target nucleotide sequence to modify (eg, cut) the sequence, whereby
   a. target cells are killed by the antibacterial agent;
   b. growth or proliferation of target cells is reduced; or
   c. target cells are sensitised to an antibiotic, whereby the antibiotic is toxic to the cells.
98. The plasmid of Clause 97, wherein the plasmid further encodes said crRNA or gRNA.
99. A host cell comprising the plasmid of any one of Clauses 95 to 98, wherein the host cell does not comprise the target nucleotide sequence.
100. The host cell of Clause 99, wherein the cell is capable of replicating the plasmid and packaging the replicated plasmid in transduction particles that are capable of infecting target bacterial cells.
101. The host cell of Clause 99 or 100, wherein the host cell comprises, integrated in the cell chromosome and/or one or more episomes of the cell,
   a. A terL;
   b. An optional terS; and
   c. Expressible nucleotide sequences encoding all structural proteins necessary for the production of transduction particles that package copies of the plasmid;
   d. wherein the chromosome and episomes of the cell (other than said plasmid) are devoid of a phage packaging sequence, wherein the phage packaging sequence comprised by the plasmid is operable together with the product of said terS and terL in the production of packaged plasmid.
102. The cell of Clause 101, wherein the terL, optional terS and nucleotide sequences encoding the structural proteins are comprised by a phage (optionally a prophage) genome in the host cell.
103. A bacterial host cell comprising the genome of a helper phage that is incapable of self-replication, optionally wherein the genome is present as a prophage, and a plasmid according to any one of Clauses 95 to 98, wherein the helper phage is operable to package copies of the plasmid in transduction particles, wherein the particles are capable of infecting bacterial target cells to which the antibacterial agent is toxic.
104. The cell of Clause 103, wherein the host cell is a cell of first species or strain and the target cells are of the same species or strain, and optionally wherein the hosts cell is an engineered cell that to which the antibacterial agent is not toxic.
105. The cell of Clause 103, wherein the host cell is a cell of first species or strain and the target cells are of a different species or strain, wherein the antibacterial agent is not toxic to the host cell.
106. A method of making a plurality of transduction particles, the method comprising culturing a plurality of host cells according to any one of Clauses 103 to 105, optionally inducing a lytic cycle of the helper phage, and incubating the cells under conditions wherein transducing particles comprising packaged copies of the plasmid are created, and optionally separating the particles from the cells to obtain a plurality of transduction particles.
107. A plurality of transduction particles obtainable by the method of Clause 106 for use in medicine, eg, for treating or preventing an infection of a human or animal subject by target bacterial cells, wherein transducing particles are administered to the subject for infecting target cells and killing the cells using the antibacterial agent.
108. A method of making a plurality of transduction particles, the method comprising
   i. Producing host cells whose genomes comprise nucleic acid encoding structural proteins necessary to produce transduction particles that can package first DNA, wherein the genomes are devoid of a phage packaging signal, wherein the expression of the proteins is under the control of inducible promoter(s);
   ii. Producing first DNA encoding an antibacterial agent or a component thereof (eg, as defined in any preceding Clause), wherein the DNA comprises a phage packaging signal;
   iii. Introducing the DNA into the host cells;
   iv. Inducing production of the structural proteins in host cells, whereby transduction particles are produced that package the DNA;
   v. Optionally isolating a plurality of the transduction particles; and
   vi. Optionally formulating the particles into a pharmaceutical composition for administration to a human or animal for medical use.
109. The method of Clause 108, wherein the DNA comprises a MGE as defined in any preceding

Clause.
110. The method of Clause 108 or 109, wherein the structural proteins are P2 phage proteins and optionally the packaging signal is a P4 phage packaging signal.
111. The method of Clause 108 or 109, wherein the DNA comprises a modified SaPI or a genomic island DNA.
112. The method of any one of Clauses 108 to 111, wherein the cells in step (iv) comprise a gene encoding a helper phage activator, optionally wherein the activator is a P4 phage delta or ogr protein when the structural proteins are P2 proteins; or the activator is a SaPI rinA, ptiA, ptiB or ptiM when the MGE comprises a modified SaPI; and optionally the expression of the activator(s) is controlled by an inducible promoter, eg, a T7 promoter.
113. The method of any one of Clauses 108 to 112, wherein the packaging signal is P4 phage Sid and/or psu; or the signal is SaPI cpmA and/or cpmB.
   This is useful for packaging DNAs into smaller capsids.
114. The method of any one of Clauses 108 to 113, wherein the cell genomes comprise prophages, wherein each prophage comprises said nucleic acid encoding structural proteins.
115. The method of Clause 114, wherein the prophages are P2 prophages devoid of cos and optionally one, more or all genes selected from int, cox orf78, B, orf80, orf81, orf82, orf83, A, orj91, tin, old, orf30 and fun(Z); and optionally the packaging signal of (ii) is a cos or P4 packaging signal.
116. The method of Clause 114 or 115, wherein the prophages are P2 prophages devoid of cos and comprising genes from Q to S, V to G and Fi to ogr.
117. The method of Clause 114, wherein the prophages are phi11 prophages devoid of a packaging signal and comprising gene 29 (terS) to gene 53 (lysin); and optionally the packaging signal of (ii) is a phi11 packaging signal.
118. A plurality of transduction particles obtainable by the method of any one of Clauses 108 to 117.
119. The particles of Clause 118 for administration to a human or animal for medical use.
   Further Concepts of the invention are as follows:—

The present invention is optionally for an industrial or domestic use, or is used in a method for such use. For example, it is for or used in agriculture, oil or petroleum industry, food or drink industry, clothing industry, packaging industry, electronics industry, computer industry, environmental industry, chemical industry, aerospace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

The present invention is optionally for use in an industry or the environment is an industrial environment, wherein the industry is an industry of a field selected from the group consisting of the medical and healthcare; pharmaceutical; human food; animal food; plant fertilizers; beverage; dairy; meat processing; agriculture; livestock farming; poultry farming; fish and shellfish farming; veterinary; oil; gas; petrochemical; water treatment; sewage treatment; packaging; electronics and computer; personal healthcare and toiletries; cosmetics; dental; non-medical dental; ophthalmic; non-medical ophthalmic; mineral mining and processing; metals mining and processing; quarrying; aviation; automotive; rail; shipping; space; environmental; soil treatment; pulp and paper; clothing manufacture; dyes; printing; adhesives; air treatment; solvents; biodefence; vitamin supplements; cold storage; fibre retting and production; biotechnology; chemical; industrial cleaning products; domestic cleaning products; soaps and detergents; consumer products; forestry; fishing; leisure; recycling; plastics; hide, leather and suede; waste management; funeral and undertaking; fuel; building; energy; steel; and tobacco industry fields.

In an example, the first DNA, first phage or vector comprises a CRISPR array that targets target bacteria, wherein the array comprises one, or two or more spacers (eg, 2, 3, 4, 5, 6, 7, 8, 9, 10, 20, 30, 40, 50 or more spacers) for targeting the genome of target bacteria.

In an example, the target bacteria are comprised by an environment as follows. In an example, the environment is a microbiome of a human, eg, the oral cavity microbiome or gut microbiome or the bloodstream. In an example, the environment is not an environment in or on a human. In an example, the environment is not an environment in or on a non-human animal. In an embodiment, the environment is an air environment. In an embodiment, the environment is an agricultural environment. In an embodiment, the environment is an oil or petroleum recovery environment, eg, an oil or petroleum field or well. In an example, the environment is an environment in or on a foodstuff or beverage for human or non-human animal consumption.

In an example, the environment is a a human or animal microbiome (eg, gut, vaginal, scalp, armpit, skin or oral cavity microbiome). In an example, the target bacteria are comprised by a human or animal microbiome (eg, gut, vaginal, scalp, armpit, skin or oral cavity microbiome).

In an example, the DNAs, phage or composition of the invention are administered intranasally, topically or orally to a human or non-human animal, or is for such administration. The skilled person aiming to treat a microbiome of the human or animal will be able to determine the best route of administration, depending upon the microbiome of interest. For example, when the microbiome is a gut microbiome, administration can be intranasally or orally. When the microbiome is a scalp or armpit microbiome, administration can be topically. When the microbiome is in the mouth or throat, the administration can be orally.

In an example, the environment is harboured by a beverage or water (eg, a waterway or drinking water for human consumption) or soil. The water is optionally in a heating, cooling or industrial system, or in a drinking water storage container.

In an example, the host and/or target bacteria are Firmicutes selected from Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter and Weisella.

In an example, the kit, DNA(s), first phage, helper phage, composition, use or method is for reducing pathogenic infections or for re-balancing gut or oral microbiota eg, for treating or preventing obesity or disease in a human or animal. For example, the first phage, helper phage, composition, use or method is for knocking-down Clostridium dificile or E coli bacteria in a gut microbiota of a human or animal.

In an example, the packaging signal, NPF and/or HPF consists or comprises SEQ ID NO: 1 or a structural or functional homologue thereof.

In an example, the packaging signal, NPF and/or HPF consists or comprises SEQ ID NO: 1 or a nucleotide sequence that is at least 70, 75, 80, 85, 90, 91, 92, 93, 94, 95, 96, 97, 98 or 99% identical thereto.

In an example, the disease or condition is a cancer, inflammatory or autoimmune disease or condition, eg, obesity, diabetes IBD, a GI tract condition or an oral cavity condition.

Optionally, the environment is comprised by, or the target bacteria are comprised by, a gut microbiota, skin microbiota, oral cavity microbiota, throat microbiota, hair microbiota, armpit microbiota, vaginal microbiota, rectal microbiota, anal microbiota, ocular microbiota, nasal microbiota, tongue microbiota, lung microbiota, liver microbiota, kidney microbiota, genital microbiota, penile microbiota, scrotal microbiota, mammary gland microbiota, ear microbiota, urethra microbiota, labial microbiota, organ microbiota or dental microbiota. Optionally, the environment is comprised by, or the target bacteria are comprised by, a plant (eg, a tobacco, crop plant, fruit plant, vegetable plant or tobacco, eg on the surface of a plant or contained in a plant) or by an environment (eg, soil or water or a waterway or aqueous liquid).

Optionally, the disease or condition of a human or animal subject is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

Neurodegenerative or Cns Diseases or Conditions for Treatment or Prevention by the Invention

In an example, the neurodegenerative or CNS disease or condition is selected from the group consisting of Alzheimer disease, geriopsychosis, Down syndrome, Parkinson's disease, Creutzfeldt Jakob disease, diabetic neuropathy, Parkinson syndrome, Huntington's disease, Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and/or reduces the progression of nerve fibre damage. In an example, the method restores nerve myelin and/or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatement and/or prevention of CNS and neurodegenerative diseases and conditions, eg, agents such as immune checkpoint inhibitors, eg, anti-PD-1, anti-PD-L1, anti-TIM3 or other antibodies disclosed therein).

Cancers for Treatment or Prevention by the Method

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pineaioma, hemangioblastoma, acoustic neuroma, oligodendroglioma, menangioma, neuroblastoma, retinoblastoma and brain metastases).

Autoimmune Diseases for Treatment or Prevention by the Method
1. Acute Disseminated Encephalomyelitis (ADEM)
2. Acute necrotizing hemorrhagic leukoencephalitis
3. Addison's disease
4. Agammaglobulinemia
5. Alopecia areata
6. Amyloidosis
7. Ankylosing spondylitis
8. Anti-GBM/Anti-TBM nephritis
9. Antiphospholipid syndrome (APS)
10. Autoimmune angioedema
11. Autoimmune aplastic anemia
12. Autoimmune dysautonomia
13. Autoimmune hepatitis
14. Autoimmune hyperlipidemia
15. Autoimmune immunodeficiency
16. Autoimmune inner ear disease (AIED)
17. Autoimmune myocarditis
18. Autoimmune oophoritis
19. Autoimmune pancreatitis
20. Autoimmune retinopathy
21. Autoimmune thrombocytopenic purpura (ATP)
22. Autoimmune thyroid disease
23. Autoimmune urticaria
24. Axonal & neuronal neuropathies
25. Balo disease
26. Behcet's disease
27. Bullous pemphigoid
28. Cardiomyopathy
29. Castleman disease
30. Celiac disease
31. Chagas disease
32. Chronic fatigue syndrome
33. Chronic inflammatory demyelinating polyneuropathy (CIDP)
34. Chronic recurrent multifocal ostomyelitis (CRMO)
35. Churg-Strauss syndrome
36. Cicatricial pemphigoid/benign mucosal pemphigoid
37. Crohn's disease
38. Cogans syndrome
39. Cold agglutinin disease
40. Congenital heart block
41. Coxsackie myocarditis
42. CREST disease
43. Essential mixed cryoglobulinemia
44. Demyelinating neuropathies
45. Dermatitis herpetiformis
46. Dermatomyositis
47. Devic's disease (neuromyelitis optica)
48. Discoid lupus
49. Dressler's syndrome
50. Endometriosis
51. Eosinophilic esophagitis
52. Eosinophilic fasciitis
53. Erythema nodosum
54. Experimental allergic encephalomyelitis
55. Evans syndrome
56. Fibromyalgia
57. Fibrosing alveolitis
58. Giant cell arteritis (temporal arteritis)
59. Giant cell myocarditis
60. Glomerulonephritis
61. Goodpasture's syndrome
62. Granulomatosis with Polyangiitis (GPA) (formerly called Wegener's Granulomatosis)
63. Graves' disease
64. Guillain-Barre syndrome
65. Hashimoto's encephalitis
66. Hashimoto's thyroiditis
67. Hemolytic anemia
68. Henoch-Schonlein purpura
69. Herpes gestationis
70. Hypogammaglobulinemia
71. Idiopathic thrombocytopenic purpura (ITP)
72. IgA nephropathy
73. IgG4-related sclerosing disease
74 Immunoregulatory lipoproteins
75. Inclusion body myositis
76. Interstitial cystitis
77. Juvenile arthritis
78. Juvenile diabetes (Type 1 diabetes)
79. Juvenile myositis
80. Kawasaki syndrome
81. Lambert-Eaton syndrome
82. Leukocytoclastic vasculitis
83. Lichen planus
84. Lichen sclerosus
85. Ligneous conjunctivitis
86. Linear IgA disease (LAD)
87. Lupus (SLE)
88. Lyme disease, chronic
89. Meniere's disease
90. Microscopic polyangiitis
91. Mixed connective tissue disease (MCTD)
92. Mooren's ulcer
93. Mucha-Habermann disease
94. Multiple sclerosis
95. Myasthenia gravis
96. Myositis
97. Narcolepsy
98. Neuromyelitis optica (Devic's)
99. Neutropenia
100. Ocular cicatricial pemphigoid
101. Optic neuritis
102. Palindromic rheumatism
103. PANDAS (Pediatric Autoimmune Neuropsychiatric Disorders Associated with Streptococcus)
104. Paraneoplastic cerebellar degeneration
105. Paroxysmal nocturnal hemoglobinuria (PNH)
106. Parry Romberg syndrome
107. Parsonnage-Turner syndrome
108. Pars planitis (peripheral uveitis)
109. Pemphigus
110. Peripheral neuropathy
111. Perivenous encephalomyelitis
112. Pernicious anemia
113. POEMS syndrome
114. Polyarteritis nodosa
115. Type I, II, & III autoimmune polyglandular syndromes
116. Polymyalgia rheumatica
117. Polymyositis
118. Postmyocardial infarction syndrome
119. Postpericardiotomy syndrome
120. Progesterone dermatitis
121. Primary biliary cirrhosis
122. Primary sclerosing cholangitis
123. Psoriasis
124. Psoriatic arthritis
125. Idiopathic pulmonary fibrosis
126. Pyoderma gangrenosum
127. Pure red cell aplasia
128. Raynauds phenomenon
129. Reactive Arthritis
130. Reflex sympathetic dystrophy
131. Reiter's syndrome
132. Relapsing polychondritis
133. Restless legs syndrome
134. Retroperitoneal fibrosis
135. Rheumatic fever
136. Rheumatoid arthritis
137. Sarcoidosis
138. Schmidt syndrome
139. Scleritis
140. Scleroderma
141. Sjogren's syndrome
142. Sperm & testicular autoimmunity
143. Stiff person syndrome
144. Subacute bacterial endocarditis (SBE)
145. Susac's syndrome
146. Sympathetic ophthalmia
147. Takayasu's arteritis
148. Temporal arteritis/Giant cell arteritis
149. Thrombocytopenic purpura (TTP)
150. Tolosa-Hunt syndrome
151. Transverse myelitis
152. Type 1 diabetes
153. Ulcerative colitis
154. Undifferentiated connective tissue disease (UCTD)
155. Uveitis
156. Vasculitis
157. Vesiculobullous dermatosis
158. Vitiligo
159. Wegener's granulomatosis (now termed Granulomatosis with Polyangiitis (GPA).

Inflammatory Diseases for Treatment or Prevention by the Method
1. Alzheimer
2. ankylosing spondylitis
3. arthritis (osteoarthritis, rheumatoid arthritis (RA), psoriatic arthritis)
4. asthma
5. atherosclerosis
6. Crohn's disease
7. colitis
8. dermatitis
9. diverticulitis
10. fibromyalgia
11. hepatitis
12. irritable bowel syndrome (IBS)
13. systemic lupus erythematous (SLE)
14. nephritis
15. Parkinson's disease
16. ulcerative colitis.

### EXAMPLES

### Example 1: Efficient Phage CRISPR Delivery Vehicle Production

### Background

We designed a strategy for efficient production of phage particles comprising components of a CRISPR/Cas system for killing target E coli Nissle strain bacteria. So our phage composition will consist of a lysate primarily containing CRISPR/Cas system components packaged in phage particles which will be devoid of phage protein-encoding sequences and which will have no or a very low proportion of helper phage. Also the strategy will work alternatively in less well characterised phage/bacterial strain combinations.

Outline of Strategy for CRISPR/Cas Component Packaging in Hitherto Unknown Phages

(i) Identify a high copy number cloning/shuttle vector (capable of cloning and propagation in a first E coli strain and then transfer to a second bacterial host strain of interest) containing an E coli ori for replication in the E coli cloning strain;
(j) Isolate temperate phage against the host (second) bacterium;
(k) Identify or engineer a phage production strain of the host bacteria that has an inactive CRISPR/Cas system (eg, a repressed Cas3 or other nuclease) and which can be infected and lysogenized with the temperate phage; or repress or inactivate the system in the production strain;
(l) In that strain make a lysogen using the temperate phage (helper phage) and test that it can be induced;
(m) Identify the packaging sequence (pac or cos) using PhageTerm (world wide web.ncbi.nlm.nih.gov/pmc/articles/PMC5557969) on whole genome sequenced phage;
(n) Delete the pac/cos packaging signal sequence in the helper phage in the host bacteria;
(o) Incorporate the packaging signal in the shuttle vector along with a CRISPR-array (and other components of the CRISPR/Cas system, such as a Cas9-encoding nucleotide sequence, orCas3 and/or Cascade-encoding sequence);
(p) Transform the vector into production host strain;
(q) UV or mitomycin C induce and harvest phage comprising the CRISPR/Cas component(s). Alternatively, use a system with inducible RecA in trans to simulate SOS (needs to be activated RecA).

Example of the Above Specifically for E coli Nissle Using Phage P2:

Nissle is useful due to its GRAS (Generally Regarded as Safe) status and P2 has a relatively broad host range (most E coli, Shigella, Klebsiella, Salmonella in addition to DNA delivery into e.g. Pseudomonas; Kahn et al 1991).

We will use pUC19 or other high copy number cloning vector. Temperate phage P2 can lysogenize Nissle. Most E coli K strains have an inactive CRISPR/Cas system and can be infected by P2 and thus all regular cloning hosts can be used (here exemplified by E coli TOP10).

P2 is introduced into TOP10 to produce a lysogen. P2 cannot be induced with mitomycin C or UV but we will use the epsilon anti-repressor from the parasite phage P4 that derepresses P2 and makes it go into lytic phase. We will express this gene from an inducible promoter in the production host strain.

The 325 bp packaging signal sequence as follows will be used

(SEQ ID NO: 1)
GCATGCGTTTTCCTGCCTCATTTTCTGCAAACCGCGCCATTCCCGGCGCG
GTCTGAGCGTGTCAGTGCAACTGCATTAAAACCGCCCCGCAAAGCGGGCG
GGCGAGGCGGGGAAAGCACCGCGCGCAAACCCAGAAGTTAGTTAATTATT
TGTGTAGTCAAAGTGCCTTGACTACATACCTCGTTAATACATTGGAGCAT
AATGAAGAAAATCTATGGCCTATGGTCCAAAACTGTCTTTTTTGATGGCA
CTATCCTGAAAAATATGCAAAAAATAGATTGATGTAAGGTGGTTCTTGTC
AGTGTCGCAAGATCCTTAAGAATTC

The packaging sequence will be deleted in the P2 prophage of the lysogenic production TOP10 strain.

A pUC19 shuttle vector encoding a guide RNA that targets the genome of the target Nissle strain (or alternatively comprising a CRISPR array for producing such a guide RNA) will be constructed and the packaging signal will be added. If the target Nissle harbours it own endogenous CRISPR/Cas system, we will use an activation strategy to activate the endogenous Cas3 by including Cas activating genes in the vector. If not, we will include an exogenous Cas3-encoding nucleotide sequence (and optionally one or more nucleotide sequences encoding one or more required Cascade components) in the vector for expression in the target Nissle. We will transform the vector into the TOP10 production strain, induce the P4 anti-repressor and harvest phage comprising the CRISPR/Cas component(s).

Since the induced (helper) phage DNA does not contain a packaging signal we will be able to isolate particles with only the vector DNA packaged. Thus, we will obtain a composition comprising such phage which can be used to infect target Nissle bacteria and introduce the CRISPR/Cas component(s) therein for killing the target bacteria.

### Example 2: MGEs, Genomics Islands Etc

Overview of possible different MGE packaging strategies follow.

Applicable to different types of phages:
Identify packaging signal and structural genes in the helper phage (delivery vehicle)
Delete packaging signal in helper phage and place on plasmid comprising MGE
Place both helper and plasmid in production strain
Induce structural gene transcription of helper to get production of helper-phage-packaged MGEs

For using parasitic mobile elements (P4 phage or SaPI etc) activation of helper phage structural genes is done by induction of a helper phage activator obtained from the parasitic element Delta in P4 or one, more or al of ptiA/B/M in SaPI.

If one wants smaller size particle one can choose to package in a parasite-size capsid (typically 10-20 kb) by including in the MGE or vector P4 Sid and psu or cpmA/B from a SaPI.

One can use defective helper phages where at least the packaging signal has been removed and structural genes are either on a plasmid or integrated as a cryptic prophage in the production host. If for some reason one cannot use this approach and need to use functional helper phages, one will include in the MGE or vector the genes on the parasite that hijack the phage packaging machinery to preferentially package parasite DNA (in our case CGV) over phage DNA.

List of the Minimal Genes One could Include on a Plasmid Vector from P4.

P4 sequence: see world wide
web.ncbi.nlm.nih.gov/nuccore/x51522
Cos packagingsite (SEQ ID NO: 3):
GCATGCGTTTTCCTGCCTCATTTTCTGCAAACCGCGCCATTCCCGGCGCG
GTCTGAGCGTGTCAGTGCAACTGCATTAAAACCGCCCCGCAAAGCGGGCG
GGCGAGGCGGGGAAAGCACCGCGCGCAAACCGACAAGTTAGTTAATTATT
TGTGTAGTCAAAGTGCCTTCAGTACATACCTCGTTAATACATTGGAGCAT
AATGAAGAAAATCTATGGCCTATGGTC

The homology between P2 and P4 pasted below; this may be used as a packaging signal in the MGE or vector:

(SEQ ID NO: 4)
TGCATTAAAACCGCCCCGCAAAGCGGGCGGGCGAGGCGGGGAAAGCACCG
CGCGC

For small capsid size (packages 11.4 kb instead of 33.5 kb) Sid and/or Psu can be included in the MGE or vector:—

Sid (SEQ ID NO: 5):
ATGTCTGACCACACTATCCCTGAATATCTGCAACCCGCACTGGCACAACT
GGAAAAGGCCAGAGCCGCCCATCTTGAGAACGCCCGCCTGATGGATGAGA
CCGTCACGGCCATTGAACGGGCAGAGCAGGAAAAAAATGCGCTGGCGCAG
GCCGACGGAAACGACGCTGACGACTGGCGCACGGCCTTTCGTGCAGCCGG
TGGTGTCCTGAGCGACGAGCTGAAACAGCGCCACATTGAGCGCGTGGCAC
GCCGGGAGCTGGTACAGGAATATGACAATCTGGCCGTGGTGCTGAATTTC
GAACGTGAACGCCTGAAAGGGGCGTGTGACAGCACGGCCACCGCCTACCG
GAAGGCACATCATCACCTTCTGAGTCTGTATGCAGAGCATGAGCTGGAAC
ACGCCCTGAATGAAACCTGTGAGGCGCTTGTCCGGGCAATGCATCTGAGC
ATTCTGGTACAGGAAAATCCGCTCGCCAACACCACCGGCCATCAGGGCTA
CGTCGCACCGGAAAAGGCTGTCATGCAGCAGGTGAAATCATCGCTGGAAC
AGAAAATTAAACAGATGCAAATCAGCCTCACCGGCGAGCCGGTTCTCCGG
CTGACCGGACTGTCAGCGGCAACACTCCCGCACATGGATTATGAGGTGGC
AGGCACACCGGCACAGCGCAAGGTGTGGCAGGACAAAATAGACCAGCAGG
GAGCAGAGCTTAAGGCCAGAGGGCTGCTGTCATGA
Psu (SEQ ID NO: 6):
ATGGAAAGCACAGCCTTACAGCAGGCCTTTGACACCTGTCAGAATAACAA
AGCAGCATGGCTGCAACGCAAAAATGAGCTGGCAGCGGCCGAACAGGAAT
ATCTGCGGCTTCTGTCAGGAGAAGGCAGAAACGTCAGTCGCCTGGACGAA
TTACGCAATATTATCGAAGTCAGAAAATGGCAGGTGAATCAGGCCGCCGG
TCGTTATATTCGTTCGCATGAAGCCGTTCAGCACATCAGCATCCGCGACC
GGCTGAATGATTTTATGCAGCAGCACGGCACAGCACTGGCGGCCGCACTG
GCACCGGAGCTGATGGGCTACAGTGAGCTGACGGCCATTGCCCGAAACTG
TGCCATACAGCGTGCCACAGATGCCCTGCGTGAAGCCCTTCTGTCCTGGC
TTGCGAAGGGTGAAAAAATTAATTATTCCGCACAGGATAGCGACATTTTA
ACGACCATCGGATTCAGGCCTGACGTGGCTTCGGTGGATGACAGCCGTGA
AAAATTCACCCCTGCGCAGAACATGATTTTTTCGCGTAAAAGTGCGCAAC
TGGCATCACGTCAGTCAGTGTAA

To activate helper phage P2, Delta can be included in a host cell genome (provided separately in a host cell, not on the MGE or vector to be packaged)

Delta (SEQ ID NO: 7):
ATGATTTACTGTCCGTCGTGTGGACATGTTGCTCACACCCGTCGCGCAC
ATTTCATGGACGATGGCACCAAGATAATGATTGCACAGTGCCGGAATAT
TTATTGCTCTGCGACATTTGAAGCGAGTGAAAGCTTTTTCTCTGACAGT
AAAGATTCAGGAATGGAATACATTTCAGGCAAACAGAGATACCGCGATT
CACTGACGTCAGCCTCCTGCGGTATGAAACGCCCGAAAAGAATGCTTGT
TACCGGATATTGTTGTCGGAGATGTAAAGGCCTTGCACTGTCAAGAACA
TCGCGGCGTCTGTCTCAGGAAGTCACCGAGCGTTTTTATGTGTGCACGG
ATCCGGGCTGTGGTCTGGTGTTTAAAACGCTTCAGACCATCAACCGCTT
CATTGTCCGCCCGGTCACGCCGGACGAACTGGCAGAACGCCTGCATGAA
AAACAGGAACTGCCGCCAGTACGGTTAAAAACACAATCATATTCGCTGC
GTCTGGAATGA

Minimum Genes to Include in the Host Chromosome/Episome from P2.

P2 sequence (acc. number: NC_001895)

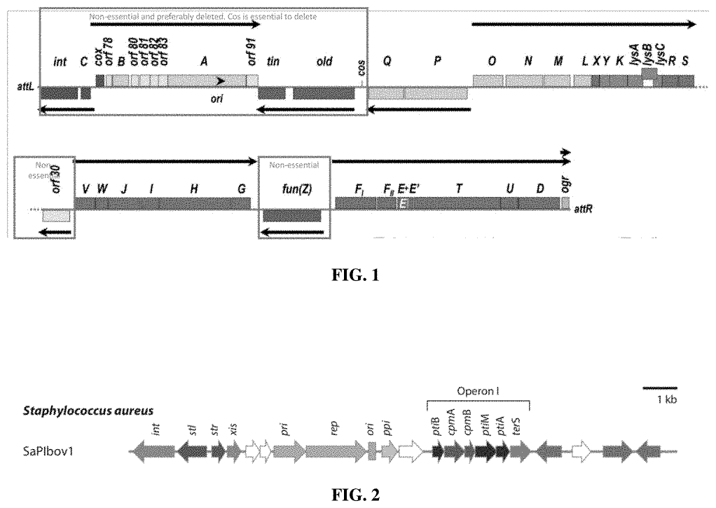

FIG. 1 shows a genetic map of P2 genome with non-essential genes boxed in red—one, more or all of these can be excluded. Cos is deleted and preferably the whole region from int through cos. This region may, for example, be swapped with a resistance marker while the orf30 and fun(Z) genes are left intact.

“Q” through “S” (SEQ ID NO: 8)
TCAGTCGTTGTCAGTGTCCAGTGAGTAGTTTTTAAAGCGGATGACCTCCTGACCGAGCCAGCCGTTTATCTCGCGGATCCTGTCCTGTAAC
GGGATAAGCTCATTGCGGACAAAGACCTTTGCCACTTTCTCAATATCACCCAGCGACCCGACGTTCTCCGGCTTGCCACCCATCAACTGAA
AGGGGATGCGGTGCGCGTCCAGCAGGTCAGCGGCGCTGGCTTTTTTGATATTAAAAAAATCGTCCTTCGTCGCCACTTCACTGAGGGGGAT
AATTTTAATGCCGTCGGCTTTCCCCTGTGGGGCATAGAGAAACAGGTTTTTAAAGTTGTTGCGGCCTTTCGACTTGACCATGTTTTCGCGA
AGCATTTCGATATCGTTGCGATCCTGCACGGCATCGGTGACATACATGATGTATCCGGCATGTGCGCCATTTTCGTAATACTTGCGGCGGA
ACAACGTGGCCGACTCATTCAGCCAGGCAGAGTTAAGGGCGCTGAGATATTCCGGCAGGCCGTACAGCTCCTGATTAATATCCGGCTCCAG
CAGGTGAAACACGGAGCCGGGCGCGAAGGCTGTCGGCTCGTTGAAGGACGGCACCCACCAGTAAACATCCTCTTCCACGCCACGGCGGGTA
TATTTTGCCGGTGAGGTTTCCAGTCTGATGACCTTACCGGTGGTGCTGTAACGCTTTTCCAGAAACGCATTACCGAACACCAGAAAATCCA
GCACAAAGCGGCTGAAATCCTGCTGGGAAAGCCATGGATGCGGGATAAATGTCGAGGCCAGAATATTGCGTTTGACGTAAATCGGCGAGCT
GTGATGCACGGCAGCCCGCAGGCTTTTTGCCAGACCGGTAAAGCTGACCGGTGGCTCATACCATCTGCCGTTACTGATGCACTCGACGTAA
TCCAGAATGTCACGGCGGTCGAGTACCGGCACCGGCTCACCAAAGGTGAATGCCTCCATTTTCGGGCCGCTGGCGGTCATTGTTTTTGCCG
CAGGTTGCGGTGTTTTCCCTTTTTTCTTGCTCATCAGTAAAACTCCAGAATGGTGGATGTCAGCGGGGTGCTGATACCGGCGGTGAGTGGC
TCATTTAACAGGGCGTGCATGGTCGCCCAGGCGAGGTCGGCGTGGCTGGCTTCCTCGCTGCGGCTGGCCTCATAGGTGGCGCTGCGTCCGC
TGCTGGTCATGGTCTTGCGGATAGCCATAAACGAGCTGGTGATGTCGGTGGCGCTGACGTCGTATTCCAGACAGCCACGGCGGATAACGTC
TTTTGCCTTGAGCACCATTGCGGTTTTCATTTCCGGCGTGTAGCGGATATCACGCGCGGCGGGATAGAACGAGCGCACGAGCTGGAACACG
CCGACACCGAGGCCGGTGGCATCAATACCGATGTATTCGACGTTGTATTTTTCGGTGAGTTTGCGGATGGATTCCGCCTGGGTGGCAAAGT
CCATGCCTTTCCACTGGTGACGCTCAAGTATTCTGAATTTGCCACCGGCCACCACCGGCGGTGCCAGTACCACGCATCCGGCGCTGTCGCC
ACGGTGTGACGGGTCGTAACCAATCCATACCGGGCGGGAGCCGAACGGATTGGCGGCAAACGGCGCATAGTCTTCCCATTCTTCCAGCGTG
TCGACCATGCAGCGTTGCAGCTCCTCGAACGGGAACACCGATGCCTTGTCGTCAACAAATTCACACATGAACAGGTTTTTAAAATCGTCGG
CGCTGTTTTCGCGTTTGAGCTGCTCAATGTCGAACAGCGTGCAGCCGCCTTTCAGGGCGTCCTCAATGGTGACAATCTGTCGCCACTGGCC
GTCCGCACAGAGAAGCCCACCGGCAAGGGCGTTATGACTGACGTCGATTTCCACGCGTTCGGCGGCGCTGGCGCGTCCCCGGTTAAACAGT
TCACCCGACCAGAACGGGTAGGCGTCGTGCGCCAGCGTGGACGGGGTGGAGAAATAGGTCGAGCGCAGGTGACTCTGTGAGGCCATACCTG
ATGCCACCTTACGCAGTACCTGAAAATTCGGGATCCAGAAAATCTCATCGACGTACAGGTCGCCGTTATGACTCTGCGCGGTGTTGGAGTT
GGTGCCGAGAAAAATCAGTTTTGCGCCGTTATTGCCCAGGACAATCGGGTCACCGGTCAGGTCAACGTCAACCAGACGGGCAAAGGCGATG
ATGTATTCGCGGAACACATACGCCTGTGTTTTACTGGCCGACAGAAAAATCTGGTTATGACCGGTTTTCAGGGCACGCAGCAGCGCCTCGC
GGGAAAAATAAAACGTCGCGCCAATCTGGCGGGATTTCAGGATATCGCGGATGCGGTGCTCAAGCCCGGCACGATACCAGTGCAGCTGATA
GTCGAAAGACTGCTCAAAGAAAATCTGCTCCAGCTTTTCGATGGCCTCGTCACTGAAAAAATTCTTTTTCGGTTTGCGCCGTCCGCCTTTG
TTACGGTTAGCGACGTTCGGATTAAGGTCTGCCTCGTTGCCGGTCTGGCTGTAGCGGTTGACCCGTGCCAGTCGTTCAATCTGGCGTCCCA
GCAGGTCAATTTCCTTGAAGTCACCGCCGGTTTTCTGTGGTTTGATGATGAGCTGGGTCAGCCGCGCTTCCAGACTCATTTCGACACGGCT
GATGGGGGCAACGCTGTCCCAGCCGTCGCGCTGTTTCCAGCTCTGCACTGTCGGGCGTTTCATCTGCAACATGGCGGCAATCTGCGGCACG
GAAAACCCCTGCCAGTACAGCAGCGCCGCCTGACGACGCGGGTCGTGTAAAAGAGTGGTGTCTGTGGTGATGGTCATGAATACCTCGCCGT
GATGAATACACGGCAAGGCTACTGAGTCGCGCCCCGCGATTCGCTAAGGTGCTGTTGTGTCAGTGATAAGCCATCCGGGACTGATGGCGGA
GGATGCGCATCGTCGGGAAACTGATGCCGACATGTGACTCCTCTAATCACTATTCAGGACTCCTGACAATGGCAAAAAAAGTCTCAAAATT
CTTTCGTATCGGCGTTGAGGGTGACACCTGTGACGGGCGTGTCATCAGTGCGCAGGATATTCAGGAAATGGCCGAAACCTTTGACCCGCGT
GTCTATGGTTGCCGCATTAACCTGGAACATCTGCGCGGCATCCTGCCTGACGGTATTTTTAAGCGTTATGGCGATGTGGCCGAACTGAAGG
CCGAAAAGATTGACGATGATTCGGCGCTGAAAGGCAAATGGGCGCTGTTTGCGAAAATCACCCCGACCGATGACCTTATCGCGATGAACAA
GGCCGCGCAGAAGGTCTACACCTCAATGGAAATTCAGCCGAACTTTGCCAACACCGGCAAATGTTATCTGGTGGGTCTGGCCGTCACCGAT
GACCCGGCAAGCCTCGGCACGGAATACCTGGAATTCTGCCGCACGGCAAAACACAACCCCCTGAACCGCTTCAAATTAAGCCCTGAAAACC
TGATTTCAGTGGCAACGCCTGTTGAGCTGGAATTTGAAGACCTGCCTGAAACCGTGTTCACCGCCCTGACCGAAAAGGTGAAGTCCATTTT
TGGCCGCAAACAGGCCAGCGATGATGCCCGTCTGAATGACGTGCATGAAGCGGTGACCGCTGTTGCTGAACATGTGCAGGAAAAACTGAGC
GCCACTGAGCAGCGCCTCGCTGAGATGGAAACCGCCTTTTCTGCACTTAAGCAGGAGGTGACTGACAGGGCGGATGAAACCAGCCAGGCAT
TCACCCGCCTGAAAAACAGTCTCGACCACACCGAAAGTCTGACCCAGCAGCGCCGCAGCAAAGCCACCGGCGGTGGCGGTGACGCCCTGAT
GACGAACTGCTGACCGGCGTCAGTCAGTCCGGGAAAACCTTCACGATTAACCCTTAATTTCAGGAAAAACTATGCGCCAGGAAACCCGCTT
TAAATTTAATGCCTACCTGTCCCGTGTTGCCGAACTGAACGGCATCGACGCCGGTGATGTGTCGAAAAAATTCACCGTTGAACCGTCGGTC
ACCCAGACCCTGATGAACACCATGCAGGAGTCCTCTGACTTTCTGACCCGCATCAATATTGTGCCGGTCAGCGAAATGAAAGGGGAAAAAA
TTGGTATCGGTGTCACCGGCTCCATCGCCAGCACTACCGACACTGCCGGTGGTACCGAGCGTCAGCCGAAGGACTTCTCGAAGCTGGCGTC
AAACAAGTACGAATGCGACCAGATTAACTTCGATTTTTATATCCGCTACAAAACGCTGGACCTGTGGGCGCGTTATCAGGATTTCCAGCTC
CGTATCCGTAACGCCATTATCAAACGCCAGTCCCTTGATTTCATCATGGCCGGTTTTAACGGCGTGAAGCGTGCCGAAACCTCTGACCGCA
GCAGCAATCCGATGTTGCAGGATGTGGCGGTCGGCTGGCTGCAGAAATACCGCAATGAAGCACCGGCGCGCGTGATGAGCAAGGTCACTGA
CGAGGAAGGCCGCACCACCTCTGAGGTTATCCGCGTGGGTAAGGGCGGTGATTATGCCAGCCTTGATGCACTGGTGATGGATGCGACCAAC
AACCTGATTGAACCGTGGTATCAGGAAGACCCTGACCTTGTGGTGATTGTGGGGCGTCAGCTACTGGCGGACAAGTATTTCCCCATCGTCA
ACAAGGAGCAGGACAACAGCGAAATGCTGGCCGCTGACGTCATCATCAGCCAGAAACGCATCGGTAACCTACCAGCGGTACGCGTCCCGTA
CTTCCCGGCGGATGCGATGCTCATCACGAAGCTGGAAAACCTGTCCATCTACTACATGGATGACAGCCATCGCCGCGTGATTGAGGAAAAC
CCGAAACTCGACCGCGTGGAGAACTACGAGTCAATGAACATTGATTACGTGGTGGAAGACTACGCCGCCGGTTGTCTGGTGGAAAAAATCA
AGGTCGGTGACTTCTCCACACCGGCTAAGGCGACCGCAGAGCCGGGAGCGTAACCGATGACGAGTCCCGCACAGCGCCACATGATGCGGGT
CTCGGCAGCGATGACCGCGCAGCGGGAAGCCGCCCCGCTGCGACATGCAACTGTCTATGAGCAGATGCTGGTTAAGCTCGCCGCAGACCAG
CGCACACTGAAAGCGATTTACTCAAAAGAGCTGAAGGCCGCAAAAAAACGCGAACTGCTGCCGTTCTGGTTGCCGTGGGTGAACGGCGTGC
TGGAGCTGGGCAAAGGTGCACAGGATGACATTCTGATGACGGTCATGCTGTGGCGTCTGGATACCGGCGATATTGCCGGTGCGCTGGAGAT
TGCCCGTTATGCCCTGAAGTACGGTCTGACCATGCCGGGTAAACACCGCCGTACCCCGCCGTACATGTTCACCGAGGAGGTAGCGCTTGCG
GCCATGCGCGCTCACGCTGCCGGTGAGTCTGTGGATACCCGCCTGCTGACGGAGACCCTTGAACTGACCGCCACGGCTGACATGCCTGATG
AAGTGCGCGCAAAGCTGCACAAAATCACCGGTCTGTTTCTGCGTGACGGTGGTGATGCCGCCGGTGCGCTGGCGCACCTGCAACGTGCGAC
ACAGCTCGACTGTCAGGCAGGCGTCAAAAAAGAGATTGAACGACTGGAGCGGGAGCTGAAACCGAAGCCGGAGCCGCAGCCCAAAGCGGCC
ACCCGCGCCCCGCGTAAGACCCGGAGCGTGACACCGGCAAAACGTGGACGCCCGAAAAAGAAAGCCAGTTAACAACCGAATGCGCCCCGCG
CCAGGGCGGCACGCCGGTCAGTGACGGTGAATCACCTGACACTGCACCGGCGTCCACCGCCCGACTTTTCAGAGGTAGTCATGATGACGCT
GATTATTCCGCGAAAGGAGGCTCCCGTGTCCGGTGAGGGTACGGTGGTCATCCCGCAACCGGCAGGCGACGAGCCGGTGATTAAAAACACG
TTCTTTTTTCCCGATATCGACCCGAAGCGCGTCCGGGAACGTATGCGCCTTGAGCAGACCGTCGCCCCCGCCCGTCTGCGTGAGGCCATCA
AGTCAGGCATGGCTGAAACGAATGCGGAGCTGTACGAGTACCGCGAACAGAAAATTGCCGCCGGTTTTACGCGTCTGGCTGACGTCCCGGC
GGACGATATCGACGGTGAAAGCATCAAGGTTTTTTACTACGAGCGCGCCGTGTGTGCGATGGCGACCGCGTCGCTTTATGAGCGTTATCGC
GGTGTGGATGCCAGTGCGAAAGGCGACAAGAAGGCTGACAGCATTGACAGCACCATTGATGAGCTGTGGCGGGATATGCGCTGGGCGGTGG
CGCGCATCCAGGGCAAGCCGCGCTGCATCGTGAGTCAAATCTGATGAAGACCTTTGCGCTACAGGGCGACACGCTCGACGCCATTTGTGTC
CGCTATTACGGGCGCACTGAGGGCGTGGTTGAGACCGTGCTCGCCGCAAATCCGGGACTGGCTGAACTGGGGGCGGTGCTGCCACACGGCA
CCGCCGTCGAACTGCCCGACGTTCAGACCGCGCCCGTGGCTGAAACTGTCAATCTGTGGGAGTAACGCATGACAGCAGAAGAAAAAAGCGT
CCTGTCGCTTTTCATGATTGGGGTGCTGATTGTTGTCGGCAAGGTGCTTGCCGGTGGTGAACCTATCACCCCGCGTCTGTTTATCGGGCGC
ATGTTGCTCGGTGGTTTTGTCTCGATGGTTGCCGGTGTTGTTCTGGTGCAGTTTCCTGACCTGTCACTGCCAGCGGTGTGCGGCATCGGCT
CCATGCTGGGTATCGCCGGTTATCAGGTGATTGAGATTGCCATTCAGCGCCGCTTTAAGGGCAGGGGGAAACAGTAATGCCGGTAATTAAC
ACGCATCAGAATATCGCCGCCTTTCTCGACATGCTGGCCGTGTCCGAAGGGACGGCGAATCATCCACTGACGAAAAACCGGGGCTATGACG
TGATAGTCACCGGACTGGACGGGAAGCCGGAAATTTTCACCGACTACAGTGACCACCCGTTCGCACATGGCCGACCGGCGAAGGTGTTTAA
CCGTCGCGGTGAAAAATCCACGGCCTCCGGTCGCTATCAGCAGCTTTACCTGTTCTGGCCGCATTACCGCAAACAGCTTGCCCTGCCGGAT
TTCAGTCCGTTGTCACAGGACAGACTCGCCATTCAGTTGATCCGCGAACGCGGAGCACTGGATGACATCCGGGCGGGACGCATTGAGCGCG
CCATTTCACGCTGTCGCAATATCTGGGCGTCCCTGCCGGGTGCCGGTTACGGTCAGCGTGAGCATTCACTGGAAAAACTGGTCACCGTCTG
GCGTACCGCTGGCGGCGTACCGGCTTAAACGGAGTAAATACCATGAAGAAATTATCCCTTTCACTGATGCTGAACGTGTCGCTGGCGCTGA
TGCTGGCACTGTCCCTGATTTACCCGCAGAGCGTGGCCGTCAATTTTGTCGCTGCCTGGGCGATTCTGGCGACGGTTATCTGTGTGGTTGC
CGGTGGTGTGGGCGTGTATGCCACTGAGTATGTGCTGGAACGCTACGGGCGGGAGCTGCCGCCGGAATCGCTGGCCGTGAAGATTGTCACG
TCGCTGTTTTTGCAGCCGGTGCCGTGGCGCAGACGGGCGGCGGCTCTGGTAGTGGTGGTGGCGACGTTTATCTCGCTGGTCGCTGCCGGGT
GGATTTTTACCGCGCTGATTTATCTTGTGGTGTCGCTGTTTTTCCGGCTGATACGTAAAGCCTGTCGTCAGCGTCTTGAGGGGCGGGAACC
ATGTCAAGGCTGATGATTGTGCTGGTCGTGTTGTTATCGCTGGCGGTGGCCGGTCTGTTTCTGGTGAAACACAAAAATGCCAGCCTGCGCG
CCTCGCTGGACAGGGCGAACAACGTCGCCAGCGGTCAGCAGACGACCATCACCATGCTGAAAAATCAGCTTCATGTTGCGCTCACCAGGGC
AGATAAAAACGAGCTGGCGCAGGTGGCACTGCGTCAGGAACTGGAGAACGCCGCGAAACGTGAAGCACAGCGCGAGAAAACCATCACGAGG
TTACTTAATGAGAACGAAGATTTTCGCCGCTGGTACGGTGCTGACCTGCCTGATGCTGTGCGCCGGTTGCACCAGCGCCCCGCCTGCACCG
ACGCCAGTGATTGTCCCCAACGCATGCCCGAAAGTGAGCCTTTGCCCGATGCCGGGCAGTGACCCGCAGACGAACGGCGATTTAAGTGCCG
ATATCCGGCAGCTTGAGAACGCGCTGGCACGCTGTGCCAGCCAGGTAAAAATGATTAAACACTGTCAGGACGAAAACGATGCTCAAACCCG
ACAGCCTGCGCAGGGCGCTGACTGATGCCGTCACGGTGCTGAAAACTAACCCCGATATGCTGCGGATATTCGTGGATAACGGGAGTATTGC
CTCCACACTGGCGGCGTCGCTGTCATTCGAAAAGCGTTACACGCTCAATGTGATTGTGACCGACTTTACCGGTGATTTTGACCTGCTCATT
GTGCCGGTGCTGGCGTGGCTGCGGGAAAATCAGCCCGACATCATGACCACCGACGAAGGCCAGAAAAAGGGCTTCACGTTTTATGCAGACA
TCAACAATGACAGCAGCTTTGATATCAGTATCAGCCTGATGCTGACCGAGCGCACGCTGGTCAGTGAGGTGGACGGCGCACTGCATGTGAA
GAATATCTCGGAACCCCCGCCGCCGGAGCCGGTCACCCGCCCGATGGAGCTGTATATCAATGGCGAACTGGTGAGTAAGTGGGATGAATGA
GTTTAAGCGTTTTGAAGACCGGCTGACCGGACTGATTGAATCGCTGTCACCGTCAGGGCGTCGGCGACTGAGTGCCGAACTGGCGAAACGT
CTGCGGCAGAGTCAGCAGCGTCGGGTGATGGCACAGAAAGCCCCGGACGGCACACCCTACGCGCCACGCCAGCAGCAGAGCGTCAGAAAAA
AGACCGGTCGCGTTAAGCGAAAAATGTTTGCGAAACTTATTACCAGTCGTTTTTTGCATATCCGTGCCAGCCCGGAGCAGGCATCAATGGA
ATTTTACGGCGGGAAGTCGCCGAAAATCGCCAGTGTGCATCAGTTTGGTCTGTCGGAAGAAAACCGGAAAGACGGTAAGAAAATTGATTAT
CCGGCGCGTCCCCTGCTCGGCTTTACCGGTGAGGATGTGCAGATGATTGAAGAGATTATCCTGGCTCACCTTGAGCGTTAG
“V” through “G” (SEQ ID NO: 9):
ATGAACACTCTCGCAAATATTCAGGAACTCGCGCGCGCACTGCGCAACATGATTCGCACTGGCATTATCGTCGAAACCGACCTTAACGCCG
GTCGCTGCCGCGTGCAGACCGGCGGCATGTGCACCGACTGGCTTCAGTGGCTGACCCATCGCGCAGGACGTTCGCGCACATGGTGGGCACC
TTCCGTGGGGGAACAGGTGCTGATTCTGGCCGTGGGTGGTGAACTCGACACGGCGTTCGTTCTGCCGGGGATTTATTCCGGCGATAACCCC
TCGCCGTCTGTGTCGGCGGATGCCCTGCATATCCGTTTCCCTGACGGGGCGGTGATTGAATATGAACCCGAAACCAGTGCACTCACGGTAA
GCGGAATTAAAACGGCCAGCGTGACGGCTTCCGGTTCTGTTACTGCCACGGTGCCGGTGGTCATGGTGAAAGCATCAACCCGCGTCACCCT
GGACACCCCGGAGGTGGTCTGCACCAACAGGCTGATTACCGGCACGCTGGAAGTGCAGAAAGGCGGGACGATGCGCGGCAACATTGAACAC
ACCGGCGGTGAACTCTCATCAAACGGTAAGGTACTGCATACCCATAAACACCCCGGCGACAGCGGCGGCACAACCGGGAGTCCTTTATGAC
AGCGCGTTATCTCGGAATGAATCGCAGTGATGGCCTGACTGTCACTGACCTTGAGCATATCAGCCAGAGTATCGGCGATATCCTGCGCACA
CCGGTCGGCTCACGGGTGATGCGTCGTGATTACGGCTCGTTGCTGGCGTCAATGATTGACCAGCCGCAGACCCCGGCGCTTGAGTTGCAGA
TTAAAGTCGCCTGTTACATGGCAGTGCTGAAATGGGAACCCCGCGTCACCCTGTCATCCGTCACCACGGCGCGCAGTTTTGACGGGCGAAT
GACGGTCACGTTAACCGGCCAGCACAACGACACCGGCCAGCCACTTTCATTAACCATCCCTGTGAGTTGAAACCATGCCGATTATCGACCT
GAACCAGCTACCCGCACCGGATGTGGTCGAGGAGCTGGACTTTGAAAGCATTCTCGCTGAACGCAAGGCGACACTGATTTCCCTTTACCCG
GAAGATCAGCAGGAGGCGGTCGCCCGTACCCTGACACTGGAATCTGAGCCTCTCGTCAAACTGCTGGAAGAAAATGCTTATCGTGAGCTTA
TCTGGCGTCAGCGTGTGAATGAGGCCGCACGGGCGGTGATGCTGGCCTGTGCCGCCGGTAATGACCTTGATGTGATTGGTGCCAATTACAA
CACCACGCGCCTGACTATCACCCCGGCAGATGATTCGACCATCCCGCCGACACCGGCAGTGATGGAATCTGACACCGATTATCGTCTGCGT
ATTCAGCAGGCTTTTGAGGGCTTAAGCGTCGCCGGGTCAGTGGGAGCCTATCAGTATCATGGTCGCAGTGCTGACGGGCGTGTCGCGGATA
TTTCTGTCACCAGTCCGTCTCCGGCCTGTGTCACCATCTCTGTGCTGTCACGTGAAAATAACGGCGTCGCATCCGAAGACCTGCTGGCTGT
GGTGCGTAACGCCCTTAATGGCGAGGACGTCAGGCCGGTGGCCGACCGCGTGACCGTGCAGTCTGCCGCCATCGTTGAATACCAGATAAAC
GCCACGCTTTACCTTTACCCTGGTCCCGAAAGCGAACCCATCCGCGCTGCCGCTGTGAAAAAGCTGGAAGCGTATATCACGGCACAGCACC
GGCTGGGGCGCGACATCCGTCTGTCTGCCATTTATGCCGCTTTGCATGTGGAAGGTGTGCAGCGTGTCGAACTGGCTGCACCACTGGCCGA
CATCGTGCTCAACAGTACGCAGGCGTCTTTCTGTACCGAATACCGCGTCGTGACCGGAGGCTCGGATGAGTGATTCGCGACTGCTGCCGAC
CGGCTCATCACCGCTTGAGGTCGCCGCCGCAAAAGCCTGTGCGGAAATTGAAAAAACGCCGGTCAGTATTCGTGAACTGTGGAACCCGGAC
ACCTGTCCGGCAAATCTGCTGCCGTGGCTGGCGTGGGCGTTTTCGGTCGACAGGTGGGATGAAAAGTGGCCGGAAGCGACAAAACGCGCCG
TTATCCGCGATGCCTATTTCATCCACTGTCATAAGGGCACGATAGGTGCAATCCGGCGTGTGGTGGAGCCGCTCGGCTATCTCATCAACGT
GACGGAGTGGTGGGAAAACAGTGACCCGCCCGGCACCTTCCGGCTTGATATTGGTGTACTGGAAAGCGGTATCACAGAGGCAATGTATCAG
GAAATGGAACGGCTGATTGCTGATGCCAAACCTGCAAGCCGTCACCTTATTGGCCTGAACATTACCCGGGACATTCCCGGCTATCTGTTCG
CCGGTGGTGTGGCTTACGACGGCGATGTAATTACGGTTTACCCCGGATAAGTGAGGAATAATGAGCATAAAATTCAGAACCGTTATCACCA
CTGCCGGTGCAGCAAAGCTGGCAGCGGCAACCGCGCCGGGAAGGCGGAAGGTCGGCATTACCACGATGGCCGTCGGGGATGGCGGTGGTAA
ATTGCCTGTCCCGGATGCCGGACAGACCGGGCTTATCCATGAAGTCTGGCGACATGCGCTGAACAAAATCAGCCAGGACAAACGAAACAGT
AATTATATTATCGCCGAGCTGGTTATTCCGCCGGAGGTGGGCGGTTTCTGGATGCGTGAGCTTGGCCTGTACGATGATGCGGGAACGTTAA
TTGCCGTGGCGAACATGGCCGAAAGCTATAAGCCAGCCCTTGCCGAAGGCTCAGGACGTTGGCAGACCTGTCGCATGGTCATCATCGTCAG
CAGTGTGGCCTCAGTGGAGCTGACCATTGACACCACAACGGTGATGGCGACGCAGGATTACGTTGATGACAAAATTGCAGAGCACGAACAG
TCACGACGTCACCCGGACGCCTCGCTGACAGCAAAAGGTTTTACTCAGTTAAGCAGTGCGACCAACAGCACGTCTGAAACACTGGCCGCAA
CGCCGAAAGCGGTAAAGGCCGCGTATGACCTGGCTAACGGGAAATATACCGCACAGGACGCCACCACAGCGCGAAAAGGCCTTGTCCAGCT
TAGTAGCGCCACCAACAGCACGTCTGAAACGCTCGCCGCAACACCAAAAGCCGTTAAGACGGTAATGGATGAAACGAACAAAAAAGCGCCA
TTAAACAGCCCTGCACTGACCGGAACGCCAACGACGCCAACTGCGCGACAGGGAACGAATAATACTCAGATCGCAAACACGGCTTTCGTTA
TGGCCGCGATTGCCGCCCTTGTAGACTCGTCGCCTGACGCACTGAATACGCTGAACGAGCTGGCGGCGGCGCTGGGCAATGACCCGAATTT
TGCTACCACCATGACTAATGCGCTTGCGGGTAAGCAACCGAAAGATGCTACCCTGACGGCGCTGGCGGGGCTTGCTACTGCGGCAGACAGG
TTTCCGTATTTTACGGGGAATGATGTTGCCAGCCTGGCGACCCTGACAAAAGTCGGGCGGGATATTCTGGCTAAATCGACCGTTGCCGCCG
TTATCGAATATCTCGGTTTACAGGAAACGGTAAACCGAGCCGGGAACGCCGTGCAAAAAAATGGCGATACCTTGTCCGGTGGACTTACTTT
TGAAAACGACTCAATCCTTGCCTGGATTCGAAATACTGACTGGGCGAAGATTGGATTTAAAAATGATGCCGATGGTGACACTGATTCATAC
ATGTGGTTTGAAACGGGGGATAACGGCAATGAATATTTCAAATGGAGAAGCCGCCAGAGTACCACAACAAAAGACCTGATGACGTTGAAAT
GGGATGCACTAAATATTCTTGTTAATGCCGTCATTAATGGCTGTTTTGGAGTTGGTACGACGAATGCACTAGGTGGTAGCTCTATTGTTCT
TGGTGATAATGATACCGGATTTAAACAGAATGGAGACGGTATTCTTGATGTTTATGCTAACAGTCAGCGTGTATTCCGTTTTCAGAATGGA
GTGGCTATTGCTTTTAAAAATATTCAGGCAGGTGATAGTAAAAAGTTCTCGCTATCCAGCTCTAATACATCCACGAAGAATATTACCTTTA
ATTTATGGGGTGCTTCCACCCGTCCAGTGGTTGCAGAGTTAGGCGATGAGGCCGGATGGCATTTCTATAGCCAGCGAAATACAGATAACTC
GGTAATATTTGCTGTTAACGGTCAGATGCAACCCAGCAACTGGGGAAATTTTGATTCCCGCTATGTGAAAGATGTTCGCCTGGGTACGCGA
GTTGTTCAATTGATGGCGCGAGGTGGTCGTTATGAAAAAGCCGGACACACGATTACCGGATTAAGAATCATTGGTGAAGTAGATGGCGATG
ATGAAGCCATCTTCAGGCCGATACAAAAATACATCAATGGCACATGGTATAACGTTGCGCAGGTGTAAGTTATGCAGCATTTAAAGAACAT
TAAGTCAGGTAATCCAAAAACAAAAGAGCAATATCAGCTAACAAAGAATTTTGATGTTATCTGGTTATGGTCCGAAGACGGAAAAAACTGG
TATGAGGAAGTGAAGAACTTTCAGCCAGACACAATAAAGATTGTTTACGATGAAAATAATATTATTGTCGCTATCACCAGAGATGCTTCAA
CGCTTAATCCTGAAGGTTTTAGCGTTGTTGAGGTTCCTGATATTACCTCCAACCGACGTGCTGACGACTCAGGTAAATGGATGTTTAAGGA
TGGTGCTGTGGTTAAACGGATTTATACGGCAGATGAACAGCAACAACAGGCAGAATCACAAAAGGCCGCGTTACTTTCCGAAGCGGAAAAC
GTTATTCAGCCACTGGAACGCGCTGTCAGGCTGAATATGGCGACGGATGAGGAACGTGCACGACTGGAGTCATGGGAACGTTACAGCGTTC
TGGTCAGCCGTGTGGATCCTGCAAATCCTGAATGGCCGGAAATGCCGCAATAA
“FI” through “ogr” (SEQ ID NO: 10)
ATGAGTGACTATCATCACGGCGTGCAGGTGCTGGAGATTAACGAGGGCACCCGCGTCATTTCCACCGTATCCACGGCCATTGTCGGCATGG
TCTGCACGGCCAGCGATGCAGATGCGGAAACCTTCCCCCTCAATAAACCTGTGCTGATTACCAATGTGCAGAGCGCAATTTCAAAGGCCGG
TAAAAAAGGCACGCTGGCGGCATCGTTGCAGGCCATCGCTGACCAGTCAAAACCGGTCACCGTTGTCATGCGCGTGGAAGACGGCACCGGT
GATGACGAGGAAACGAAACTCGCGCAGACCGTTTCCAATATCATCGGCACCACCGATGAAAACGGTCAGTACACCGGACTAAAAGCCATGC
TGGCGGCGGAGTCGGTAACCGGTGTTAAACCGCGTATTCTCGGCGTGCCGGGACTGGATACCAAAGAGGTGGCTGTTGCACTGGCATCAGT
CTGTCAGAAGCTGCGTGCTTTCGGGTATATCAGCGCATGGGGCTGTAAAACCATTTCCGAGGTGAAAGCCTATCGTCAGAATTTCAGCCAG
CGTGAGCTGATGGTCATCTGGCCGGATTTCCTCGCATGGGATACGGTCACCAGTACCACCGCCACCGCGTATGCCACCGCCCGTGCGCTGG
GGCTGCGCGCTAAAATCGACCAGGAGCAGGGCTGGCATAAAACGCTGTCCAATGTCGGGGTGAACGGTGTTACCGGCATCAGCGCATCTGT
ATTCTGGGATTTGCAGGAGTCCGGCACCGATGCTGACCTGCTTAACGAGTCAGGCGTCACTACGCTGATTCGCCGCGACGGTTTCCGCTTC
TGGGGTAACCGTACCTGCTCTGATGACCCGCTGTTCCTCTTTGAAAACTACACCCGCACCGCGCAGGTCGTGGCCGACACGATGGCTGAGG
CGCACATGTGGGCGGTGGACAAGCCCATCACTGCAACGCTGATTCGCGACATCGTTGACGGCATCAATGCCAAATTCCGTGAGCTGAAAAC
AAACGGCTATATCGTGGATGCGACCTGCTGGTTCAGCGAAGAATCCAACGATGCGGAAACCCTCAAGGCCGGAAAACTGTATATCGACTAC
GACTATACACCGGTGCCTCCTCTCGAAAACCTGACCCTGCGCCAGCGTATTACCGATAAATACCTGGCAAATCTGGTCACCTCGGTTAACA
GCAATTAAGGAGCCTGACCGATGGCAATGCCGCGCAAACTCAAGTTAATGAACGTCTTTCTGAACGGCTACAGCTATCAGGGCGTTGCAAA
GTCCGTCACGCTGCCAAAACTGACCCGTAAGCTCGAAAACTATCGCGGTGCGGGGATGAACGGCAGCGCACCGGTAGACCTCGGCCTTGAT
GACGATGCGCTGTCAATGGAGTGGTCGCTCGGTGGCTTCCCGGATTCGGTTATCTGGGAGCTTTACGCCGCAACCGGTGTGGATGCCGTGC
CGATTCGTTTTGCAGGCTCTTACCAGCGCGACGATACCGGCGAAACGGTGGCCGTCGAAGTGGTCATGCGTGGACGTCAGAAAGAAATCGA
CACCGGCGAGGGTAAACAGGGAGAAGACACTGAGTCGAAAATCTCCGTGGTCTGCACCTATTTCCGGCTGACGATGGACGGTAAGGAGCTG
GTCGAAATTGACACCATCAACATGATTGAGAAGGTGAACGGCGTCGATCGGCTGGAGCAACACCGCCGCAATATCGGCCTGTGATTTTCAT
CCGGTCAGCCTGGCTGGCCGGTTAACCCTGATTCAGAAGTGAGAAAACCATGAACAAAGAAAATGTCATTACCCTGGACAATCCGGTCAAA
CGTGGTGAGCAGGTTATCGAACAGGTCACGCTGATGAAACCCAGTGCCGGGACGCTACGCGGTGTCAGTCTGGCTGCGGTTGCAAACTCCG
AAGTCGATGCACTGATTAAGGTGCTGCCGCGCATGACGGCACCGATGCTGACCGAGCAGGAAGTCGCCGCGCTGGAACTGCCTGACCTTGT
GGCGCTGGCCGGTAAGGTGGTCGGTTTTTTGTCGCCGAACTCGGTGCAGTGACGTTTCCGAAAAATCTCTCGGTCGATGACCTGATGGCGG
ATGTGGCAGTGATATTTCACTGGCCGCCATCAGAACTGTATCCCATGAGCCTGACCGAACTCATCACATGGCGCGAAAAGGCGCTCCGGCG
AAGCGGAAACACGAATGAGTAACAATGTAAAATTACAGGTATTGCTCAGGGCTGTTGACCAGGCATCCCGCCCGTTTAAATCCATCCGCAC
AGCGAGCAAGTCGCTGTCGGGGGATATCCGGGAAACACAAAAATCACTGCGCGAGCTGAACGGTCACGCATCCCGTATTGAGGGATTCCGC
AAGACCAGTGCACAGCTCGCCGTGACTGGTCATGCACTTGAAAAGGCACGGCAGGAGGCCGAAGCCCTTGCCACACAGTTTAAAAACACCG
AACGTCCGACCCGTGCTCAGGCGAAAGTCCTGGAATCCGCAAAGCGTGCGGCGGAGGACTTACAGGCGAAATATAACCGCCTGACAGATTC
CGTTAAACGCCAGCAGCGGGAACTGGCCGCTGTGGGAATTAATACCCGCAATCTTGCACATGATGAGCAGGGACTGAAAAACCGTATCAGT
GAAACCACCGCACAGCTTAACCGTCAGCGTGATGCGCTGGTGCGTGTCAGTGCGCAACAGGCAAAACTTAACGCAGTAAAACAGCGTTATC
AGGCCGGAAAGGAACTGGCCGGAAATATGGCCTCAGTGGGCGCTGCCGGTGTGGGGATTGCGGCGGCGGGAACGATGGCCGGTGTTAAGCT
ACTGATGCCCGGTTATGAGTTTGCGCAGAAAAACTCAGAATTACAGGCTGTGATCGGAGTGGCAAAAGACTCCGCCGAAATGGCCGCACTC
CGCAAGCAGGCGCGCCAGCTCGGCGACAATACCGCCGCCTCGGCAGATGATGCAGCCGGTGCGCAGATTATTATTGCGAAAGCCGGTGGGG
ATGTTGATGCCATTCAGGCGGCAACGCCGGTCACGCTGAACATGGCGCTGGCGAACCGTCGCACAATGGAAGAAAACGCCGCCCTGCTGAT
GGGGATGAAATCCGCCTTTCAGCTTTCAAACGATAAGGTCGCTCATATCGGGGATGTTCTCTCCATGACGATGAACAAAACCGCCGCCGAT
TTTGACGGCATGAGCGATGCGCTGACCTATGCCGCACCTGTGGCAAAAAATGCCGGTGTCAGCATTGAAGAAACCGCCGCAATGGTCGGGG
CGCTGCATGATGCAAAAATCACAGGCTCAATGGCGGGGACGGGAAGCCGTGCCGTGTTAAGCCGCCTGCAGGCACCGACGGGAAAAGCATG
GGATGCACTCAAAGAGCTTGGAGTGAAAACCTCAGACAGCAAAGGAAACACCCGGCCAATATTTACCATTCTGAAAGAAATGCAGGCCAGT
TTTGAGAAAAACCGGCTCGGTACTGCCCAGCAGGCTGAATACATGAAAACTATTTTCGGGGAGGAGGCCAGCTCAGCCGCTGCCGTGCTGA
TGACTGCCGCCTCAACCGGAAAGCTGGACAAACTGACCGCTGCGTTTAAAGCCTCAGACGGGAAGACCGCCGAGCTGGTAAATATCATGCA
GGACAACCTAGGCGGTGACTTTAAAGCGTTTCAGTCCGCTTATGAGGCGGTGGGGACTGACCTGTTTGACCAGCAGGAAGGCGCGCTGCGT
AAGCTCACGCAGACGGCCACAAAGTATGTGTTAAAACTCGACGGCTGGATACAGAAAAACAAATCACTGGCGTCAACCATCGGCATCATTG
CCGGCGGTGCACTGGCGCTTACTGGCATCATCGGTGCCATTGGCCTCGTAGCCTGGCCGGTTATCACCGGCATCAATGCCATCATCGCGGC
AGCAGGCGCAATGGGGGCAGTCTTCACGACGGTTGGCAGTGCTGTTATGACCGCCATCGGGGCTATTAGCTGGCCGGTTGTGGCCGTGGTG
GCTGCCATTGTCGCCGGTGCGTTGCTTATCCGTAAATACTGGGAGCCTGTCAGCGCATTCTTTGGTGGTGTGGTTGAAGGGCTGAAAGCGG
CATTTGCGCCGGTGGGGGAACTGTTCACGCCACTTAAACCGGTTTTTGACTGGCTGGGCGAAAAGTTACAGGCCGCGTGGCAGTGGTTTAA
AAACCTGATTGCCCCGGTCAAAGCCACCCAGGACACCCTGAACCGTTGCCGTGACACGGGCGTCATGTTCGGGCAGGCACTGGCTGACGCG
TTGATGCTGCCGCTTAATGCGTTCAACAAACTGCGCAGTGGTATTGACTGGGTACTGGAAAAACTCGGTGTTATCAACAAAGAGTCAGACA
CACTTGACCAGACCGCCGCCAGAACTCATACCGCCACGTATGGTACCGGTGACTATATTCCGGCGACCAGCTCTTATGCAGGCTATCAGGC
TTATCAGCCGGTCACGGCACCGGCTGGCCGCTCTTATGTAGACCAGAGTAAAAACGAATATCACATCAGCCTGACGGGGGGGACTGCGCCG
GGGACACAGCTTGACCGCCAGTTACAGGATGCGCTCGAAAAATACGAGCGGGATAAACGTGCGCGCGCCCGTGCCAGCATGATGCATGACG
GTTAAGGAGGTGACGAAAAATGATGCTCGCGTTAGGTATGTTTGTTTTTATGCGCCAGACGCTGCCACACCAGACCATGCAGCGTGAATCA
GATTATCGCTGGCCGTCAAATTCCCGTATCGGTAAACGGGATGCCTTTCAGTTTCTCGGTGTGGGTGAGGAAAACATCACGCTGGCCGGTG
TGCTTTATCCCGAACTGACCGGCGGCAAGCTGACGATGACCACGCTCAGGCTGATGGCAGAGGAGGGGCGGGCGTGGCCGTTGCTGGATGG
CACCGGCATGATTTACGGCATGTATGTCATCAGCAGGGTGAGTGAAACAGGGAGTATTTTCTTTGCAGACGGCACACCCCGGAAAATTGAT
TTTACGCTGTCACTCACCCGCGTTGATGAATCACTGGCCGCGCTTTATGGCGATATCGGTAAACAGGCGGAATCGCTCATCGGTAAGGCCG
GCAGTATGGCGACCAGATTCACAGGTATGACGGGGGCGGGATAATGCTGGATGCGCTGACATTTGATGCAGGCAGTACGCTGACGCCGGAT
TACATGCTGATGCTCGACAGCAGGGATATTACCGGCAATATCAGCGACCGTCTGATGAGCATGACCCTGACGGATAACCGGGGCTTTGAGG
CTGACCAGCTTGATATTGAACTGAACGATGCCGACGGGCAGGTCGGGCTGCCGGTTCGTGGCGCTGTCCTGACGGTGTATATCGGCTGGAA
AGGTTTTGCCCTGGTATGCAAAGGGAAATTTACCGTTGATGAGGTTGAACACCGGGGCGCACCGGATGTAGTCACCATCCGCGCCCGGAGT
GCAGATTTTCGCGGGACGCTCAATTCCCGCCGGGAAGGCTCCTGGCATGACACCACGCTCGGTGCGATTGTTAAGGCGATAGCCACCCGTA
ACAGGCTGGAAGCCAGTGTCGCTCCGTCACTGGCCGGAATAAAAATTCCACACATCGACCAGTCGCAGGAGTCTGATGCGAAATTCCTGAC
CCGTCTTGCAGAACGCAACGGCGGTGAGGTGTCGGTAAAAATGGGAAAACTGTTGTTTCTCAAAGCGGGGCAGGGAGTGACGGCCAGCGGT
AAAAAAATCCCGCAGGTCACCATAACCCGCAGCGACGGCGACCGCCATCATTTTGCGATTGCTGACCGTGGAGCCTACACCGGTGTAACGG
CAAAATGGCTACACACTAAAGACCCGAAGCCGCAAAAGCAGAAGGTAAAACTGAAACGCAAAAAGAAAGAGAAACACCTGCGCGCACTGGA
GCACCCGAAAGCGAAACCGGTCAGGCAGAAGAAAGCGCCTAAAGTACCGGAAGCGCGTGAAGGTGAATACATGGCCGGTGAGGCTGACAAC
GTTTTTGCCCTGACCACGGTATATGCCACGAAAGCGCAGGCCATGCGCGCCGCTCAGGCGAAGTGGGATAAACTGCAACGGGGCGTTGCGG
AGTTCTCTATCAGCCTGGCTACCGGTCGGGCAGATATTTACACGGAAACACCGGTCAAAGTGTCTGGCTTTAAGCGCGTCATAGACGAGCA
GGACTGGACAATCACTAAGGTGACACATTTTCTGAATAATAGCGGCTTCACGACGTCCTTAGAGCTTGAGGTCAGGCTTTCTGATGTGGAG
TACGAAACAGAAGATGATGAGTGATGTTTTTGTTTTATCTGTTTGTTTTGTAAGGATAAATTAACTAAAATGGCACCATCAACAAAACCGG
AAGAGGTGCTCGCGATGTTTCATTGTCCTTTATGCCAGCATGCCGCACATGCGCGTACAAGTCGCTATATCACTGACACGACAAAAGAGCG
TTATCATCAGTGCCAGAACGTGAATTGCAGCGCCACGTTCATCACTTATGAGTCGGTACAGCGATACATCGTGAAGCCGGGAGAAGTCCAC
GCCGTAAGGCCGCACCCGTTGCCATCAGGGCAGCAAATTATGTGGATGTAA

Minimal Genes to Include from a SaPI on a Vector or MGE.

Several different SaPI systems exist. FIG. 2 is exemplified one of the well characterized SaPIs (SaPIbov1), which exploits phages phi11 or phi80alpha as helper phage. SaPIbov1 sequence (acc. number: AF2 7235.1)

### Packaging Signal

If one uses a defective helper phage with deleted packaging signal one can use that signal from the helper phage. In this example from S. aureus phi11 (acc. number: AF424781), as follows:

(SEQ ID NO: 11)
ANGATTTANTCC

For small capsid size (packages 15.8 kb instead of 43.6 kb), one can include cpmA and/or cpmB in the MGE or vector.

cpmA (SEQ ID NO: 12)
MKTESYFKEYNQFVLDQHKAIQELEQERNALESKIKLDKSTYKQLIMDGQ
DDKADNLYQATDADEKKLKALNKRLETKKSVSKEVKYQKTIELLKHQSEL
SSLYESEKQSAIEKLKKAVDAYNEIIDEIEDINDRYEDEHQQYASVYSQE
QLYDDKEARKALNGHFKENIFTSFINGNDLPYEHNNKLFLKC
cpmB (SEQ ID NO: 13):
MKTKYELNNTKKVANAFCLNEEDTNLLINAVDLDIKNNMQEISSELQQAE
QSKQKQYGTTLQNLAKQNRIIK

To activate helper phage phi11 one can include one, more or all of ptiA, B and M (provided separately in a host cell and not on the MGE or vector to be packaged)

ptiA (SEQ ID NO: 14)
MDKQQIKDFVCDYHERTRSDVLIDDDINTDEFFSIADENSNEWMADDNID
DHIVKNHLEMIVDRVANDKEFYIFDSLIQGRSYQDISGVLDCSEQSVRFW
YETLLDKIVEVIE
ptiB (SEQ ID NO: 15)
MESIAEKETYHLPTEHLQVFNVIKNTSNKYITKTKILNQLGYEYNSSNER
WLRRVINSLVYDYGYPIGCSYKPSERGYYIITTEQEKQQAMRSIKKLADG
SMKRYEALKRIEV
ptiM (SEQ ID NO: 16):
MIAYPIRVGS VYRGEQMKLLKTKNCLYYRNGDNKLSEYQLLTQFNPTFI
NKKIRMCEFQIESMYHMSASTTTCDEMMGVVSVSYPIEKLVIKIIETKAR
LQNYKNRSISNMVLLKTVLNHYTEKEQKKVVKYMRSNGRYKPYNVIERLQ
VDLYQASIKQRSERQKQRNIAIENSKIARVNAYHQSSYVKVV

Minimum genes to include in the host chromosome/episome from phi11.

Phi11 sequence (acc.number: AF424781)
gene #29 (terS) through gene #53 (lysin)
(SEQ ID NO: 17)
atgaacgaaaaacaaaagagattcgcagatgaatatataatgaatggatgtaatggtaaaaaagcagcaattacagcaggttatagtaagaa
aacagcagagtctttagcaagtcgattgttaagaaatgttaatgtttcggaatatattaaagaacgattagaacagatacaagaagagcgtt
taatgagtattacagaagctttagcgttatctgcttctattgctagaggagaacctcaagaggcttacagtaagaaatatgaccatttaaac
gatgaagtggaaaaagaggttacttacacaatcacaccaacttttgaagagcgtcagagatctattgaccacatactaaaagtacatggtgc
gtatatcgataaaaaagaaattactcagaagaatattgagattaatattggtgagtacgatgacgaaagttaaattaaactttaacaaaccg
tctaatgattcaatagaaacatattcgaaatactaaccaattacgataacttcactgaagtacattacggtggaggttcgagcggtaagtct
cacggcgttatacaaaaagttgtactcaaagcattgcaagattggaaatatcctaggcgtatactgtggcttagaaaagtacaatcaacaat
taaagatagtttgttcgaagatgttaaagattgtttgataaactttggtatttgggacatgtgcctttggaataagactgataacaaagttg
aattgccaaacggcgcagtttttttgtttaaaggattagataacccagagaaaataaagtcgataaaaggcatatcagacatagtcatggaa
gaagcgtctgaattcacactaaatgattacacgcaattaacgttgcgtttgagggagcgtaaacacgtgaataagcaaatatttttgatgtt
taacccagtatctaaactgaattgggtttataagtatttctttgaacatggtgaaccaatggaaaatgtcatgattagacaatctagttatc
gagataataagtttcttgatgaaatgacacgacaaaacttagagttgttagcaaatcgtaatccagcatattacaaaatttatgcgttaggt
gaatttgctacactagacaaattggttttccctaagtatgaaaaacgtttaataaataaagatgagttaagacatttaccttcttattttgg
attggactttggctacgttaatgatcctagtgcttttatacattctaaaatagatgtaaagaaaaagaagttatacatcattgaagagtatg
ttaaacaaggtatgctgaatgatgaaatagctaatgtcataaagcaacttggttatgctaaagaagaaattacagcagatagtgcagaacaa
aaaagtatagctgaattaaggaatctagggcttaaaaggattttaccaaccaaaaaagggaagggctcggttgtacaagggttacaattctt
aatgcaatttgaaatcattgttgatgaacgttgtttcaagactattgaagagatgacaactacacatggcaaaaggacaaagatacaggtga
atataccaatgaaccagtagatacatacaatcattgtatcgattcgttgcgttattcagtggaacgattctacagaccggttagaaaacgca
caaatctcagttcgaaagttgacacaataaaatctctaggattataggagggaacaaatgttaaaagtaaacgaatttgaaacagatacaga
tctacggggaaacataaattacttatttaatgatgaagccaatgttgtttacacatatgacgggacggaatccgatttattacaaaacgtta
atgaagtaagtaaatacattgaacatcacatggattaccaacgacctagattgaaagtgaaagtgattattacgaaggtaaaactaagaact
tagagagttaacacgacgcaaagaagagtacatggcagataaccgtgtagcgcatgattacgcatcttatattagcgattttatcaacggct
atttcttgggtaatccaattcaatatcaagatgatgacaaagatgtattagaagttattgaggcgttcaatgatttaaatgatgttgagtca
cacaatagatctttaggattagatttgtcaatttatggcaaagcttatgagttaatgattagaaaccaagatgatgaaacgcgtttatacaa
gagtgatgcaatgagtacttttgtcatatacgacaatacaattgaacgtaatagtatcgcaggcgttagatatttaagaactaaaccaatag
acaagactgacgaagatgaagtgtttacagttgatttattcacttcacacggtgtttatagatatcttaccagtagaacaaatggattgaag
ctcacaccacgtgaaaacggattgaatcacactattcgaacgtatgcctattacagaatttagcaacaacgaaagaagaaaaggggattatg
agaaagtaatcactttaattgatttgtatgataatgctgaatcagatactgctaactatatgagtgatttaaatgacgctatgttacttatt
aaaggtaatttaaatttagatcctgtagaagttagaaaacaaaaggaagctaacgtgttgtttttagaaccgactgtttatgctgatagcga
aggtagagaaacagaaggctctgttgatggtggttatatttataagcaatacgatgtacaaggtaccgaagcttataaagaccgtttaaaca
gtgatatacacatgtttaccaacacgcctaacatgaaagatgataactttagcggcactcaatcgggcgaggcaatgaaatacaaattattt
ggattggaacaacgtactaaaactaaagaaggattgtttactaaagggttaagacgtcgtgctaagttgttagagacaatacttaaaaatac
atggtcgattgacgctaacaaagatttcaatactgttagatacgtatacaacagaaacttacctaaatcattgattgaagaattaaaagctt
atattgattctggtgggaagattagccaaacaactttaatgtctctattctcgttcttccaagaccctgaattagaagttaagaaaatcgaa
gaagatgagaaagaatctattaaaaaagctcaaaaaggtatttataaagaccctagagacatcaatgatgacgaacaagatgatgatacaaa
agatactgttgataaaaaggaatgattgtaattgcctaacaaaaacactcaagaatattgggaagaacgcggacgcaaagcaatcgagaatg
agttgaagcgtgataaaactaaagctgaagaaatagaacgtatattgaatatgatgattaagcgcattgaaaaagagatcaatgcgtttatt
gtcaagtacggagattttgcaggcgttacattacaagaagcacaaaagattattgatgagttcgatgtaaaagcgtttcaagaagaagcaaa
aagattggtcgaaaacaaggagtttagcgatagagcaaatgaagaattaaagaagtataacacgaaaatgtatgtatctagagaacagatgt
taaagattcaaatagaattcttaattgcttatgcaacagctcaaacagaattatcgatgagggaatatttcgaatcaacagcttatcgtgtg
ttcagtgatcaagcgggtattttaggtgaaggtgtacaagtagctaaagaagttatagatacaatcgttgatacacaatttcatggtgtcgt
ttggtcagagcgattatggactaataccgaagcaatgaaacaagaagtagaagaaataattgctaatgtagttattagaggtcgacatccta
atgaatatgttaaagatatgcgcaagcacttaaataaattcgaaggcacagcacgacaaaagaccgcagcaattaaatcattgctttatacg
gaatcggcacgtgttcacgcacaatcaagcattgacagcatgaaagaaatttcaccggaaggatattatatgtatattgcaaaaatcgataa
tagaacaactaaagtatgcaaagggcttaatggagaaatattcaaagttaaagacgctaaaattggtgttaatttctatcctatgcatatca
attgtcgttcagattgcgctttactacctaaatctatgtggccgaaaaaaccaagcaagaaacgaaaaacaaaatacttcggagggaaagtg
aaaagcggtgattgatttaaaagtgaagttttttaaaggcaagttagttttgtatgacagtaaattaaatgtttggaggatactaatatgag
taatactgacaaataccttagagacatagcaagagaattaaaaggtatacgtaaagagttacaaaagcgaaacgaaacagttattattgatg
caaacttagacagtttaaggtcggcagtattagccgataaagaaaaatcgaaatataatgaacctctcttttaatagctagcacttaattgt
gttggctattttttatgtccaaaacgtgctgatgacataaaaagcacgcatggaaaaacagtcgacagactataaatggaggtatatctcat
ggaagaaaataaacttaagtttaatttgcaattttttgcagaccaatcagatgatccggacgaaccaggcggagatggtaaaaaaggaaatc
ctgataagaaagaaaatgacgaaggtactgaaataactttcacgccagagcaacaaaagaaagttgatgaaatacttgaacgtcgtgtagcc
cacgaaaagaaaaaagctgatgagtatgcaaaagaaaaagcagcagaagctgctaaagaagctgctaaattagcgaaaatgaacaaggatca
aaaagatgaatatgaacgcgaacaaatggaaaaagaactggaacaattacgttcagaaaaacaattaaacgaaatgcgttcagaagcacgaa
aaatgttgagtgaagcggaagttgattcatcagatgaggttgtcaatttagttgtaacagatactgctgaacaaactaaattgaatgttgaa
gctttttctaatgcagtaaaaaaagcggttaatgaagcggttaaggttaacgctagacaatcgccattgactggtggagattcatttaatca
ctcgactaaaaataaaccgcaaaacttagctgaaatagctagacaaaaaagaattattaaaaattaacggaggcatttaaatggaacaaaca
caaaaattaaaattaaatttgcaacattttgcaagtaacaatgttaaaccacaagtatttaaccctgacaatgtaatgatgcatgaaaagaa
agatggcacgttgttaaacgactttacaacacctatcttacaagaggttatggaaaactctaaaatcatgcaattaggtaagtacgaaccaa
tggaaggtactgagaagaagtttactttttgggctgataaaccaggtgcttactgggtaggtgaaggtcaaaaaatcgaaacgtctaaggct
acttgggttaatgctacaatgagagcgtttaaattaggggttatcttaccagtaacaaaagaattcttgaattacacttattcacaattctt
tgaagaaatgaaacctatgattgctgaagctttctataaaaagtttgacgaggcaggtattttgaatcaaggtaacaatccgttcggtaaat
caattgcacaatcaattgaaaaaactaataaggttattaaaggtgacttcacacaagataacattattgatttagaggcattgcttgaagat
gacgaattagaagcaaatgcatttatctcaaaaacacaaaacagaagcttgttacgtaaaattgtagatcctgaaacgaaagaacgtattta
tgaccgtaacagtgattcgttagacggtctacctgtggttaaccttaaatcaagcaacttaaaacgtggtgaattaatcactggtgacttcg
acaaattgatttatggtatccctcaattaatcgaatacaaaatcgatgaaactgcacaattatctacagttaaaaacgaagatggcacacct
gtaaacttgtttgaacaagacatggtggcattacgtgcaactatgcatgtagcattgcatattgctgatgataaagcgtttgctaagttagt
tcctgctgacaaaagaacagattcagttccaggagaagtttaataaataattaggagtggtaacatgcccgaaatcattggaattgttaaag
tagattttacagatttagaagataacagacatgtctatatgaaagggcatgtctaccctcgtaaaggttataatcctacagatgaacgtatc
aaagctttagctagtgttgaaaataaacgcaacaaacaaatgatttacattgtaaatgacaaattaaccaaaaaagaacttgtcgaaatagc
aagtgttgctggcttacaagttgatgaaaaacaaacaaaagctgaaattatcaatgcttttgagtcactagagtaggtggttatatgactac
gctagctgatgtaaaaaaacgtattggtcttaaagatgaaaagcaagatgaacaattagaagaaatcataaaaagttgtgaaagccagttgt
tatcaatgttacctattgaagttgaacaaataccggaaaggtttagttacatgattaaagaagttgcagttaaacgctacaacaggattggt
gctgaaggtatgacatcagaagcggttgacggacgtagcaatgcgtatgaattgaacgatttcaaggagtatgaagctattattgataatta
ctttaatgctagaacgagaactaaaaaaggaagggctgtgttcttttgagatatgaagatagagttatttttcaattagaacaagtagcaac
ttacaatcctaaaactagcaaaaaagaaaacacactaatcacttatgatgcgataccatgcaatattaaccccatttctagagcaagaaagc
aacttgaatttggtgatgtaaaaaacgatgtaagtgttctgaggataaaagaatcaatatcttaccctgttagccacgtgttggttaatggc
attcgctacaagatagttgatacaaggatatacagacacgaaacgtcatattatatcgaagaggtcaattgatgaatatagatggattagac
gcactgttaaaccaatttcacgatatgaaaaccaacattgatgatgatgtagatgatattttacaggaaaacgccaaagaatatgtagtacg
agctaaattgaaagctagagaagtaatgaataagggttattggactggtaatttatcacgcaatatcagatataaaaaaactggcgatttgc
aatacactatcacatcgcacgcagcttatagtggtttcttagaatttggtactcgatacatggaggctgaaccttttatgtggccggtatac
gaagtgattaggaaatcaactgtagaagaattgaaagcgttgtttgaataggagataaaagcatgacaccgaacttacaactttataataaa
gcgtatgaaacgctacaaggatatggattccctgttatttctcgtaaagagatgcaacaagagattccgtatcctttttttgtaataaaaat
gccggagtcaaatagaagtaagtacacgtttgatagttattctggcgatacgaatttagttattgatatttggagtgtaagcgatgatttag
gacatcatgacggacttgttaaaagatgtattgatgatttaacacctagcgttaaaacaaacgattatgactttgaagaagaagatactaac
atcacacagttagttgatgatactaccaatcaagaattgctacacacatcagtaacgatatcttacaaaacattttaaaaaacggaggaata
ttgaatggcaaatatgaaaaatagtaatgatcgtattattttatttagaaaagctggcgaaaaagtagatgctactaaaatgctttttttaa
ctgaatacggcttatcacatgaagctgatacagatacagaggatacaatggacggttcttataacactggtggttctgttgagtcaacaatg
tctggtactgctaaaatgttttatggtgacgattttgcagatgaaattgaagatgcagttgtagatcgcgtattgtatgaagcttgggaagt
tgaaagtagaataccaggcaaaaatggggattccgctaaatttaaagcgaaatatttccaaggtttccacaataaatttgaattaaaagcag
aagctaacggtattgatgaatatgaatatgaatatggagtgaatggtcgtttccaacgtggatttgcaacactacctgaggctgtaacaaag
aaacttaaggcgactggatacagattccacgacactacaaaagcagatgcattaactggcgaagatttaacagcaattccacaacctaaagt
agattcaccaccggttgcaccaagagaggtataaaaatagggcgttaagccctttttattttgtttaaattaattatgaatggagattttaa
gttatgaatgtagaaattaacggaaagtcattagaattaagttttggttttaaatttttaagagaaatcgataaccgattaggtttaaaagt
tgaacaagcttctatcggtcaaggtgtatcaatgttgcctgtaggtttagaaagtggaaatccggttgtgattggcgaagttttaatcgcag
ctacatctcacttaaaaaaacaagcaattactattaataacattgatgaagcattagatgaaatcgcagaaaatatcggactagaagaattc
ggttcggatattttaacggagttgggaaagcgacctatgacccgaaacctagtcgaagtagtggaaacggaagaaaaaccagcggaagccta
ataacttacgacagaatcgttataacttgtatgtcaacacttggtattacagatttgaacgttattgagcaaatgacattaacagaatataa
ctatcgaatgtatgcgaaagagtatgaaatgctaacccaagaattcgaacgttacaaacttgcgtttgctattcgtgatgctgcagctacta
aaaatgttgggacagaaaataaacctaaagaggaatatgtttttaacaatgcaaacgacgtattgccttatgaagaaaatatccaacggctt
aacgaaggtaaagatataagatttagtagcgaacgtgatgaatacgaaccacaaaataatgaattctttaaagttatagcagaatttaataa
gcaatagaaagagaggtgttaatgtgacggaatataaaattaaagcgactattgaagctagtgtagccaaattcaaaaggcaaattgatagt
gcggttaagtctgtgcaaagatttaaacgagtagcagatcaaactaaagatgttgaattaaacgctaacgataaaaatttacaaaaaactat
caaagagctaaaaagtcatagatgcctttagtaacaaaaatgtaaaagctaaattagatgctagtatacaagatttacaacaaaaggtacta
gaatcgaattagaactagacaaactaaactctaaagaagttacaccagaagttaagagcaaaaacaaaagttgattaaagatatcgctgaaa
cagaagctaaattatcagaattagaaaagaaacgtgtcaatattgacgtcaatgcagataacagtaaattcaatcgagtgttaaaagtatct
aaagctagtctcgaagcattaaataggtctaaagccaaagctattatagacgtggacaatggtgttgctaactctaaaatcaaacgtactaa
agaagaacttaaaagtattccgaacaaaactagatctcgacttgatgtagatacagggctactataccaactatttatgcgtttaaaaaatc
attagacgcattgccgaacaaaaaaacaacaaaggtagatgtcgatactaatggtttaaagaaagcttatgcctacataataaaagcaaatg
acaattacaaagacagatggggaatttagctaatatgaccgtgtgacggtactgtaggactaatatggaggtggattacttacatcatcttt
tagtatcttaatacctgtaatagcgagcgtagtacctgtagtatttgcgctattaaacgctatcaaagtgttaactggtggtgtacttgctt
taggtggtgccgtagcaatagcgggagcaggatttgtagcgtttggcgcaatggctatcagcgctataaagatgcttaatgatggcacttta
caagctagctcagcaacaaacgaatacaaaaaagcgttagatggcgtaaagtcagcatggactgatattataaagcaaaatcaatccgctat
cttcacaactcttgcaaatggtttaaatactgttaaaactgcaatgcagagcttacaaccgttttttagtggtatttcaagaggaatggaag
aagcgtctcaaagcgtgcttaaatgggctgaaaatagcagtgtagcttcaagattctttaatatgatgaatacaacgggtgtttcggtattt
aacaagctattaagtgctgcaggtggttttggtgacggattagtcaatgtattcacgcaattagcaccactgtttcaatggtcggctgattg
gttggatagattaggtcaatctactctaactgggctaatagtgcagctggagaaaattcgataactcgattattgaatacacaaaaacaaac
ttacctatcattggtaatattacaaaaatgattcgaggaattaacaatttgatgaatgcattcagcggatcatcaactggcatattccaatc
tcagaacaaatgacagctaagtttagggaatggtctgaacaagtaggacaatctcaagggtttaaagactttgtcagttatatacaaacaaa
tggaccactaataatgcaattgattggaaacatcgcaagaggattagagcattcgcaacagcaatggctcctatagctagtgcagtattacg
cgagcagagcaataactggttggatagctaacttgtttgaggcgcatccagctacagcacaattagttggtgtcattataactttagttggt
gcatttagatttttaataccgattattcagctgtatctaacatatgggtggcggattaataggtagaatcattgcattagtaagtaagacgg
atattaagagcgggattaacaattttaaaaggtgcgttcatgttattaaaaggaccattaaaaattatatcagttatattccaattgttatt
cggtaagattggattaattagaaatgctatcacaggactagtaactgtgtttggtattttaggcggtccaataacaatagtaattggtgtaa
ttgctgcattaatagctatattcgttttattgtggaataaaaatgaaggattcagaaactttattataaatgcttggaatgcgataaaaacg
tttatggttaatgtttggaatgtattaaaagctgtagcttcggagtatggaatgctatataacagctatcactacagcagtatcgaatgata
caatatataatgattgatggaatcaaatagtcgcttatttacaagggctatggaatggaattatcgctattgcaacaacagtatggaacctt
ttagttacaatcattacaactgttttcacgacgataatgacaatagttatgacgatatggacagctatttggacgttcttaagtacaatctg
gaatacgataattacaatcgctactacgatttggaatttgttagtcactgtaataactacagtgtttaccacaattatgactatcgcaataa
caatttggaacgctatttggacgttcttacaaacgttgtggaacactatagttactgtggcaactaaggtttggaacgctatcactacagct
atatctactgcgttacaagcggcatggagttttatttctaatatatggaatacgatttggagtttcttatctggtatattaacgacaatttg
gaataaagttgtaagcatattcacacaagttgtttcaactatatcagacaaaatgtctcaagcttggaacttcattgtcactaaaggtatgc
aatgggtatctactataacaagtacgctaattaactttgttaatagagttgttcaaggattcgttaatgttgtaaacaaagttagtcaaggt
atgacaaatgcagtaaataaagttaaaagctttgtggatgactttgtatcagcaggtgctgatatgatccgtggtttgatgagaggtattgg
taatatggctagagacttagctgaaaaagcagctagtgtagcaaaaggtgctttaaatgcagccaaaagagcgctaggtattcactcacctt
cacgtgaattcatggatgttggtatgtattcaatgttaggtttcgttaaaggtatagataatcattcaagtaaagttatccgtaatgtttct
aatgttgcagataaagtagttgatgcatttcaacctacattaaacgcacctgacatttctagtattacaggaaacttaagtaatttaggtgg
aaatataaatgcgcaagtacaacacacacattctattgaaacatcaccgaacatgaaaactgttaaagttgaattcgatgtcaataacgatg
cgcttactagtattgttaacggcagaaatgctaaacgcaattctgagtattacttataaaggaggttacaaatggacatagaattaacaaaa
aaagatggtactgtaatcaaattaagtgaatacgggtttatcgttaacgatatagtaattgatagcatgcaaatcaacacaaagtatcaaga
caaagaaaatatgaacggtcgtatattaatggggagcaattatatcagtagagatatagttgttccttgtttttgtaaagttaaaaatcgtt
cagacattgcttatatgcgagatatgttgtattcgttaacgacagacatagaacctatgtatttgcgagaaatcagaagaaaagaagagttg
aattacaggtttactcaaccaacttctgatgattacgtgaaattagataaaaacaacttcccggattacgaatattcaagacacgatcaaca
aatttatgtaaatggtaaacagtataaagttatttttaacggagttataaaccctaaacaaaaaggtaataaagtttcttttgaactaaaat
tcgaaactacagaattaccatacggtgaaagtattggaacaagcctagagttagaagaaaacaaaaaggttggattgtggtcgtttgatttt
aatattgattggcatgcaggcggagacaaaagaaagtatacatttgaaaatttgagcaaaggtacagtttactatcatggtagtgctcctaa
cgaccaattcaacatgtataaaaagataacaattattttaggcgaagatacagaatcgtttgtatggaatttaacgcatgctgaaataatga
aaatcgaagggatcaaactaaaagctggagacagaattgtttatgatagcttccgagtttataaaaacggtgttgaaataagtaccgaaacg
aatatagcccaaccaaaatttaaatacggagctaataaatttgagtttaatcaaacggtacaaaaagttcagtttgatttgaaattttatta
taagtaggtgtcagaatgacaataactattaaaccacctaaaggtaatggcgcacctgtaccagtagaaacaactttagtaaaaaaagttaa
tgctgacggtgtattaacttttgatattctagaaaataaatatacttatgaagttattaacgctatagggaaaagatggattgttagtcatg
tcgaaggtgaaaacgacaagaaagaatatgtaataactgtcattgataggaaatcagaaggcgacagacaactggttgaatgtactgctaga
gagattcccatagacaagttaatgattgatagaatttatgttaatgtaacaggatcttttacagtagaaagatattttaacattgtgtttca
aggtactggaatgctttttgaagtcgagggcaaagttaaatcttcaaagtttgaaaatggtggtgaaggcgatacaaggttagaaatgttta
aaaagggattagaacatttcggtttagaatataaaataacgtatgacaaaaagaaagacagatataagtttgtattgacgccttttgcaaat
caaaaagcgtcttattttatttctgacgaagtcaacgccaacgctataaaactcgaggaagatgcaagtgatttcgccaccttcattagagg
atatggtaattattcaggagaagaaacattcgaacacgctgggctcgtaatggaagctagaagtgcattagctgaaatatacggcgacatcc
acgcagaaccatttaaagatggtaaagtgactgaccaagaaactatggataaagaattacaatcgagattgaaaaagtcgttaaaacaatct
ttgtctttggactttttggtgttaagagaatcatatccagaagcagacccacaacccggagacatagtacaaataaaatctaccaaactagg
tttgaatgatttagtccgtatagtacaagttaaaacgattaggggtataaacaatgtaattgttaagcaagatgtaacgcttggtgagttta
atcgagaacaacgatatatgaaaaaagttaatactgcagctaactatgtttctggattaaatgatgttaacctttctaatcctagtaaagcg
gcagaaaacttgaagtctaaagtagcgtcaatagctaaatcaacactcgatttgatgagtagaactgatttgattgaagataaacaacagaa
ggtaagctctaaaactgtgactacatctgacggcactatcgttcatgattttatagataaatcaaacattaaagatgtaaaaacgattggaa
cgattggcgattctgtagctagaggatcacatgcgaaaactaatttcacagaaatgttaggcaagaagttaaaagctaaaacgaccaacctt
gcaagaggtggcgcaacaatggcaacagttccaataggtaaagaagcggtagaaaacagcatttatagacaagcagagcaaataagaggaga
cctaatcatattacaaggtacagatgatgactggttacatggttattgggcaggcgtaccgataggcactgataaaaccgacactaaaacgt
tttacggcgccttttgttctgcaattgaagttatcaggaaaaataatccagcttcaaaaatacttgtaatgacagctactaggcaatgccct
atgagtggtacaacgatacgccgtaaagatacggacaaaaacaaactagggttaactttagaggattatgtcaatgctcagatattggcttg
tagtgaattggatgtaccagtatatgatgcttatcacacagattatttcaaaccatataatccagcatttagaaaatctagcatgcctgatg
gattacatcctaatgaaagaggtcatgaagttattatgtatgagcttattaaaaattattatcagttttatggatagtaaaggaggaaaaca
tgagtaataaactaattacagatttaagtagagtctttgactacagatatgtagatgaaaatgagtataactttaaacttatttcagacatg
ctgacggattttaatttctctcttgaataccacagaaataaagaggtattcgcacatgatggagaacaaataaagtatgaacatttaaatgt
tacaagtaacgtctctgactttttaacatatttaaacggtcgatttagcaacatggtactaggtcataacggcgacggtatcaacgaagtaa
aagacgcgcgcgttgataatacaggttatggtcataagacattgcaagatcgtttgtatcatgattattcaacactagatgttttcactaaa
aaggttgagaaagctgtagatgaacactataaagaatatcgagcgacagaataccgattcgaaccaaaagagcaagaaccggaatttatcac
tgatttatcgccatatacaaatgcagtaatgcaatcattttgggtagaccctagaacgaaaattatttatatgacgcaagctcgtccaggta
atcattacatgttatctagattgaagcccaacggacaatttattgatagattgcttgttaaaaacggcggtcacggtacacacaatgcgtat
agatacattgatggagaattatggatttattcagctgtattggacagtaacaaaaacaacaagtttgtacgtttccaatatagaactggaga
aataacttatggtaatgaaatgcaagatgtcatgccgaatatatttaacgacagatatacgtcagcgatttataatccggtagaaaatttaa
tgatttttagacgtgaatataaacccactgaaagacaacttaagaattcgttgaactttgttgaggttagaagtgctgacgatattgataaa
ggtatagacaaagtattgtatcaaatggatatacctatggaatacacttcagatacacaacctatgcaaggtatcacttatgatgcaggtat
cttatattggtatacaggtgattcgaatacagccaaccctaactacttacaaggcttcgatatcaaaacgaaagaattgttatttaaacgtc
gcatcgatataggcggtgtgaataacaactttaaaggagatttccaagaggctgagggtctagatatgtattacgatctagaaacaggacgt
aaagcacttctaatcggggtaactattggacctggtaacaacagacatcattcaatttattctatcggtcaaagaggtgtaaaccaattctt
gaaaaacatcgcacctcaagtatcaatgactgattcaggcggacgtgttaaaccgttaccaatacagaacccagcatatctaagtgatatta
cggaagttggtcattactatatctatacgcaagacacacaaaatgcattagatttcccgttaccgaaagcgtttagagatgcagggtggttc
ttggatgtactgcctggacactataatggtgctctaagacaagtacttaccagaaacagcacaggtagaaatatgcttaaattcgaacgtgt
cattgacattttcaataagaaaaacaacggagcatggaatttctgcccgcaaaacgccggttattgggaacatatccctaagagtattacaa
aattatcagatttaaaaatcgttggtttagatttctatatcactactgaagaatcaaaccgatttactgattttcctaaagactttaaaggt
attgcaggttggatattagaagtaaaatcgaatacaccaggtaatacaacacaagtattaagacgtaataacttcccgtctgcacatcaatt
tttagttagaaactttggtactggtggcgttggtaaatggagtttattcgaaggaaaggtggttgaataatggtagtagataatttttcgaa
agatgataacttaatcgagttacaaacaacatcacaatataatccggttattgacacaaacatcagtttctatgaatcagatagaggaactg
gtgttttaaattttgcagtaactaagaataacagacccttatctataagttctgaacatgttaaaacatctatcgtgttaaaaaccgatgat
tataacgtagatagaggcgcttatatttcagacgaattaacgatagtagacgcaattaatgggcgtttgcagtatgtgataccgaatgaatt
tttaaaacattcaggcaaggtgcatgctcaggcattctttacacaaaacgggagtaataatgttgttgttgaacgtcaatttagcttcaata
ttgaaaatgatttagttagtgggtttgatggtataacaaagcttgtttatatcaaatctattcaagatactatcgaagctgtcggtaaagat
tttaaccaattaaagcaaaatatggctgatacacaaacgttaatagcaaaagtgaatgatagtgcgacaaaaggcattcaacaaatcgaaat
caagcaaaacgaagctatacaagctattactgcgacgcaaactagtgcaacacaagctgttacagctgaattcgataaaatagttgaaaaag
agcaagcgatttttgaacgtgttaacgaagttgaacaacaaatcaatggcgctgaccttgttaaaggtaattcaacaacgaattggcaaaag
tctaaacttacagatgattacggtaaagcaattgaatcgtatgagcagtccatagatagcgttttaagcgcagttaacacatctaggattat
tcatattactaatgcaacagatgcgccagaaaagacggatataggcacgttagagaagcctggacaagatggtgttgatgacggttcttcgt
tcgatgaatcaacttatacatcaagcaaatctggtgtgttagttgtttatgttgttgataataatactgctcgtgcaacatggtacccagac
gattcaaacgatgagtacacaaaatacaaaatctacggcacatggtacccgttttataaaaagaatgatggaaacttaactaagcaatttgt
tgaagaaacgtctaacaacgctttaaatcaagctaagcagtatgtagatgataaattcggaacaacgagctggcaacaacataagatgacag
aggcgaatggtcaatcaattcaagttaacttaaataatgcgcaaggcgatttgggatatttaactgctggtaattactatgcaacaagagtg
ccggatttaccaggtagcgttgaaagttatgagggttatttatcggtattcgttaaagatgatacaaacaagctatttaacttcacacctta
taactctaaaaagatttacacacgatcaatcacaaacggcagacttgagcaacagtggacagttcctaatgaacataaatcaacggtattgt
tcgacggtggcgcaaatggtgtaggtacaacaatcaatctaactgaaccgtacacaaactattctattttgttggtaagtggaacttatcca
ggtggcgttattgagggattcggactaaccgcattacctaacgcgattcaattgagtaaagcgaatgtagttgactcagacggcaacggtgg
cggtatttatgagtgcttactatccaaaacaagtagcactactttaagaatagataacgatgtgtactttgatttaggtaaaacatcaggtt
ctggagcgaatgccaacaaagttactataactaaaattatggggtggaaataatgaaaatcacagtaaacgataaaaacgaagttatcggat
tcgttaatactggcggtttacgcaatagtttagatgtagatgataacaatgtgcctattaaatttaaagaagagttcgaacctagaaagttt
gttttcactaacggcgaaattaaatacaatagcaatttcgaaaaagaagacgtaccgaatgcatcaaaccaacaaagtgcgtcagatttaag
tgatgaggaacttcgcggaatggttgcgagtatgcaaatgcaggtggcacaagtaaacgtattaacaatggaattagctcaacaaaacgcta
tgttaacacaacagttgactgaactgaaaactaacaaaacaagtactgagggggacgtttaaataatgaagatgatttatccaacttttaaa
gacattaaaactttttatgtttggggttactataaaaacgagcaaattaagtggtacgtagacaagggtttaatcgataaagaagaatacgc
tttaatcactggagaaaaatatccagaaacaaaagatgaaaagtcacaggtgtaatgcttgtggctttttaatttgaataaagtgggtggca
taatgtttggatttaccaaacgacatgaacaagattggcgtttaacgcgattagaagaaaatgataagactatgtttgaaaaattcgacaga
atagaagatagtcttagagcgcaagaaaagatttatgacaaattagatagaaattttgaagaattaaagcgcgacaaggtagaagatgaaaa
gaataaagaaaagaatgccaagaatattagagacataaaaatgtggattctaggtttgatagggactatcttcagtacgattgtcatagctt
tactaagaactgtttttggtatttaaaggaggtgattaccatgcttaaagggattttaggatatagcttctgggcgtgcttctggtttggta
aatgtaaataacagttaagagtcagtgcttcggcactggctttttattttgattgaaatgaggtgcatacatgggattacctaatccgaaaa
atagaaagcccacagctagtgaagtggttgaatgggcgttatatatcgctaaaaacaaaatagctattgatgtacctggttctggaatggga
gcacaatgctgggatttacctaattatttactcgataaatattgggggtttagaacatggggaaatgctgatgctatggctcaaaaatccaa
ttatagaggtagagatttcaagataattagaaatacaaaagattttgtaccacaaccaggcgactggggtgtttggactggtggttgggcag
gacatgtaaacattgtagtgggaccatgcacaaaagactattggtatggcgtagatcaaaactggtatacaaataacgcaacaggaagtcca
ccttataaaattaaacactcttatcatgatggaccaggtggaggggttaaatattttgttagaccaccatatcatccagacaaaactacacc
ggcacctaaaccagaagatgatagtgatgataacgaaaaaaataataaaaaagttccaatttggaaagatgtaacaactataaagtacacta
tttctagccaagaggttaattatccagaatatatttatcactttatagtagaaggtaatcgacgactcgaaaaacctaaaggaataatgatt
agaaacgcacaaacgatgagctcggtagaaagtttatataacagtaggaagaaatacaaacaggatgtagaatatccccacttttatgttga
tagacataatatttgggcacctagaagagctgtatttgaagttcctaatgaacctgattatatagttatagacgtatgtgaagattatagtg
cgagtaaaaatgaatttatttttaatgagattcacgcaatggttgtagctgtagatatgatggccaaatatgagatacctctaagtattgaa
aatttaaaagtagacgacagcatttggcgttcaatgttggaacatgttaattggaatatgattgacaacggtgttcctcctaaagataaata
cgaagcattagaaaaggcattacttaatatatttaaaaacagagaaaaattattaaattctataactaagccaacagtaacaaaatctagaa
taaaagttatggtagataataaaaacgctgatatagctaatgtaagagactcgtcaccaacagccaacaatggttcggcatctaaacaaccg
cagatcataacagaaacgagtccttatacattcaaacaagcactggataaacaaatggcaagaggtaacccgaaaaaatctaatgcttgggg
ttgggctaacgctacacgagcacaaacgagttcagcaatgaatgtaaagcgtatatgggaaagtaacacacaatgctaccaaatgcttaatt
taggcaagtatcaaggtgtttcagttagcgcacttaataagatacttaaaggtaagggaacattgaataatcaaggtaaagcgttcgcagaa
gcttgtaaaaagcacaacattaatgaaatttatttaatcgcgcatgctttcttagaaagtggatatggaacaagtaacttcgctaacggaaa
agatggagtatacaactacttcggcattggcgcttacgacaacaatcctaactacgcaatgacgtttgcaaggaataaaggttggacatctc
cagcaaaagcaatcatgggcggtgctagcttcgtaagaaaggattacatcaataaaggtcaaaacacattgtaccgaattagatggaatcct
aagaatccagctacccaccaatacgctactgctatagagtggtgccaacatcaagcaagtacaatcgctaagttatataaacaaatcggctt
aaaaggtatctacttcacaagggataaatataaataaagaggtgtgtaaatgtacaaaataaaagatgttgaaacgagaataaaaaatgatg
gtgttgacttaggtgacattggctgtcgattttacactgaagatgaaaatacagcatctataagaataggtatcaatgacaaacaaggtcgt
atcgatctaaaagcacatggcttaacacctagattacatttgtttatggaagatggctctatattcaaaaatgagccccttattatcgacga
tgttgtaaaaggtttccttacctacaagatacctaaaaaggttatcaaacacgctggttatgttcgctgtaagctgtttttagagaaagaag
aagaaaaaatacatgtcgcaaacttttctttcaatatcgttgatagtggtattgaatctgctgtagcaaaagaaatcgatgttaaattggta
gatgatgctattacgagaattttaaaagataacgcgacagatttattgagcaaagactttaaagagaaaatagataaagatgtcatttctta
catcgaaaagaatgaaagtagatttaaaggtgcgaaaggtgataaaggtgaaccgggacaacctggagcaaaaggtgaagcaggtaaaaaag
gagaacaaggcgcacccggtaaaaacggtactgtagtatcaatcaatcctgacactaaaatgtggcaaattgatggtaaagatacagatatc
aaagcagaacctgagttattggacaaaatcaatatcgcaaatgttgaagggttagaaaataaattgcaagaagttgaaaaaatcaaagatac
aactctcaacgactctaaaacgtatacggatacaaaaattgctgaactagttgatagcgcgcctgaatctatgaacacattaagagaattag
cagaagcaatacaaaacaactctatttcagaaagtgtattgcaacagattggctcaaaagttaatacagaagattttgaggaattcaaacaa
acactaaatgatttatatgctccaaaaaatcataatcatgacgagcggtatgttttgtcatctcaagcttttactaaacaacaagcggataa
tttatatcaactaaaaagcgcatctcaaccgacggttaaaatttggacaggaacagaaaatgaatataactatatatatcaaaaagacccga
atacgttatatttaattaaagggtgatttttatggaaggtaattttaaaaatgtaaagaagtttatttacgaaggtgaagaatatacaaaag
tatatgctggaaatatccaagtatggaaaaagccttcatcttttgtaataaaacccttacctaaaaataaatatccggatagcatagaagaa
tcaacagcaaaatggacaataaatggagttgaacccaataaaagttatcaggtgacaatagaaaatgtacgtagcggtataatgaggatttc
gcaaactaatttagggtcaagtgatttaggaatatcaggagtcaatagcggagttgcaagtaaaaatatcaactttagtaatccttcaggga
tgttgtacgtcactataagtgatgtttattcaggatctccgacattgaccattgaataattttaaacgactaatttttagtcgtttttttat
tttggataaaaggagcaaacaaatggatattaactggaaattgagattcaaaaacaaagcagtactaactggtttagttggagcattgttgc
tatttatcaagcaagtcacggatttattcggattagatttatctactcaattaaatcaagctagcgcaattataggcgctatcctcacgtta
cttacaggtattggcgttattactgacccaacgtcaaaaggcgtctcagattcatctatagcacagacatatcaagcgcctagagatagcaa
aaaagaagaacaacaagttacgtggaaatcatcacaagacagtagtttaacgccggaattaagcgcgaaagcaccaaaagaatatgatacat
cacaacctttcacagacgcctctaacgatgttggctttgatgtgaatgagtatcatcatggaggtggcgacaatgcaagcaaaattaactaa
aaatgagtttatagagtggttgaaaacttctgagggaaaacaattcaatgtggacttatggtatggatttcaatgctttgattatgccaatg
ctggttggaaagttttgtttggattacttctaaaaggtttaggtgcaaaagatattccgttcgctaacaacttcgacggattagctactgta
taccaaaatacaccggacttcttagcacaacctggcgacatggtggtattcggtagcaactacggtgctggatatggtcacgttgcatgggt
aattgaagcaactttagattacatcattgtatatgagcagaattggctaggcggtggctggactgacggaatcgaacaacccggctggggtt
gggaaaaagttacaagacgacaacatgcttatgatttccctatgtggtttatccgtccgaattttaaaagtgagacagcgccacgatcagtt
caatctcctacacaagcacctaaaaaagaaacagctaagccacaacctaaagcagtagaacttaaaatcatcaaagatgtggttaaaggtta
tgacctacctaagcgtggtagtaaccctaaaggtatagttatacacaacgacgcagggagcaaaggggcgactgctgaagcatatcgtaacg
gattagtaaatgcacctttatcaagattagaagcgggcattgcgcatagttacgtatcaggcaacacagtttggcaagccttagatgaatca
caagtaggttggcataccgctaatcaaataggtaataaatattattacggtattgaagtatgtcaatcaatgggcgcagataacgcgacatt
cttaaaaaatgaacaggcaactttccaagaatgcgctagattgttgaaaaaatggggattaccagcaaacagaaatacaatcagattgcaca
atgaatttacttcaacatcatgccctcatagaagttcggttttacacactggttttgacccagtaactcgcggtctattgccagaagacaag
cggttgcaacttaaagactactttatcaagcagattagggcgtacatggatggtaaaataccggttgccactgtctctaatgagtcaagcgc
ttcaagtaatacagttaaaccagttgcaagtgcatggaaacgtaataaatatggtacttactacatggaagaaagtgctagattcacaaacg
gcaatcaaccaatcacagtaagaaaagtggggccattcttatcttgtccagtgggttatcagttccaacctggtgggtattgtgattataca
gaagtgatgttacaagatggtcatgtttgggtaggatatacatgggaggggcaacgttattacttgcctattagaacatggaatggttctgc
cccacctaatcagatattaggtgacttatggggagaaatcagttagaatgacatagtcatgtctatttaagcaggtgcgttacatacctgct
ttctatttacatttaaagataaaatgtgctattattttactagaactttttaacatttctctcaagatttaaatgtagataacaggcaggta
ctacggtacttgcctatttttttatgcaaattttaaaaaacactttactaataaacatttgtttagtataattatatttgtaggttagttga
tgacttacaaattatgtgtaaggaggtgaaaagcctcatgctagacataataaaaacacttctagaacatcaagtattggcagtactgataa
ttccagaagtgttaaaacaacttagagaatggcatctcggctacctagaccgaaagccaaacaacaaagattaacattatgcttggagcctg
atggctcctccttacacttatataatataatattatttggaggttttcaattatgacagaacaaatgtatttaatattgtttttattaagcc
taccattgttattatttatcgggagaaagacacatttttattgtttagataaaaagaatggacgtagataatatgagtgattataaattaaa
aataattgaattgatcaaaagtgatataacaggttaccaaattcacaaacaaactggcgtagcgcaatatgtaatttcacaattaaggcaag
gaaagcgcgaagtagataacttaactttaaatacaactgaaaaactatacagttacgcacgacaagtgttataatataaatgtgaaatggtc
attcttgaaatgactcggtcgctactggcacagaccgtttaaagtgtcaccacaacatgaactgagaattcatatgacgttgctgacgagcg
acaaagctctgtgttcctgaatgggagtaggtttgtgtggtggtataatttagtaacagcatagactgtctatagcaaagttgccgaagaga
ttctaaacgtatttataaatacgtggcccttgctagataaccgcatcttaactgatgcggttatttttatccccacacaaccaacaaaacca
caccacctattaatttaggagtgtggttgttttaatatgtgaagctaaaataactacaaatgataccatttttgataccattttgttgtaaa
acagaaaaaataaggaaaataaaaaaggcaaaaaaacgcattaaatcaacgtttattgtctcatgaaatttaaatgtatataaatttca

A List of Phage that Work with SaPIs

Different SaPIs are linked to different helper phages (see FIG. 3 below)

One can mutates the helper phage to only contain structural genes to direct the phage to package in smaller capsids. If only looking at the genes responsible for small capsid packaging (cpmA and cpmB) these are highly conserved among staphylococci indicating that they will function to redirect packaging in a variety of phages broader than the list below (FIG. 3).

TABLE 1
Example Bacteria
Optionally, the host cells are selected from this Table and/or the target
cells are selected from this Table (eg, wherein the host and target cells
are of a different species; or of the same species but are a different
strain or the host cells are engineered but the target cells are wild-type
or vice versa). For example the host cells are E coli cells and the target
cells are C dificile, E coli, Akkermansia, Enterobacteriacea,
Ruminococcus, Faecalibacteriium, Firmicutes, Bacteroidetes, Salmonella,
Klebsiella, Pseudomonas, Acintenobacter or Streptococcus cells.

Abiotrophia
Abiotrophia defectiva
Acaricomes
Acaricomes phytoseiuli
Acetitomaculum
Acetitomaculum ruminis
Acetivibrio
Acetivibrio cellulolyticus
Acetivibrio ethanolgignens
Acetivibrio multivorans
Acetoanaerobium
Acetoanaerobium noterae
Acetobacter
Acetobacter aceti
Acetobacter cerevisiae
Acetobacter cibinongensis
Acetobacter estunensis
Acetobacter fabarum
Acetobacter ghanensis
Acetobacter indonesiensis
Acetobacter lovaniensis
Acetobacter malorum
Acetobacter nitrogenifigens
Acetobacter oeni
Acetobacter orientalis
Acetobacter orleanensis
Acetobacter pasteurianus
Acetobacter pornorurn
Acetobacter senegalensis
Acetobacter xylinus
Acetobacterium
Acetobacterium bakii
Acetobacterium carbinolicum
Acetobacterium dehalogenans
Acetobacterium fimetarium
Acetobacterium malicum
Acetobacterium paludosum
Acetobacterium tundrae
Acetobacterium wieringae
Acetobacterium woodii
Acetofilamentum
Acetofilamentum rigidum
Acetohalobium
Acetohalobium arabaticum
Acetomicrobium
Acetomicrobium faecale
Acetomicrobium flavidum
Acetonema
Acetonema longum
Acetothermus
Acetothermus paucivorans
Acholeplasma
Acholeplasma axanthum
Acholeplasma brassicae
Acholeplasma cavigenitalium
Acholeplasma equifetale
Acholeplasma granularum
Acholeplasma hippikon
Acholeplasma laidlawii
Acholeplasma modicum
Acholeplasma morum
Acholeplasma multilocale
Acholeplasma oculi
Acholeplasma palmae
Acholeplasma parvum
Acholeplasma pleciae
Acholeplasma vituli
Achromobacter
Achromobacter denitrificans
Achromobacter insolitus
Achromobacter piechaudii
Achromobacter ruhlandii
Achromobacter spanius
Acidaminobacter
Acidaminobacter hydrogenoformans
Acidaminococcus
Acidaminococcus fermentans
Acidaminococcus intestini
Acidicaldus
Acidicaldus organivorans
Acidimicrobium
Acidimicrobium ferrooxidans
Acidiphilium
Acidiphilium acidophilum
Acidiphilium angustum
Acidiphilium cryptum
Acidiphilium multivorum
Acidiphilium organovorum
Acidiphilium rubrum
Acidisoma
Acidisoma sibiricum
Acidisoma tundrae
Acidisphaera
Acidisphaera rubrifaciens
Acidithiobacillus
Acidithiobacillus albertensis
Acidithiobacillus caldus
Acidithiobacillus ferrooxidans
Acidithiobacillus thiooxidans
Acidobacterium
Acidobacterium capsulatum
Acidocella
Acidocella aminolytica
Acidocella facilis
Acidomonas
Acidomonas methanolica
Acidothermus
Acidothermus cellulolyticus
Acidovorax
Acidovorax anthurii
Acidovorax caeni
Acidovorax cattleyae
Acidovorax citrulli
Acidovorax defluvii
Acidovorax delafieldii
Acidovorax facilis
Acidovorax konjaci
Acidovorax temperans
Acidovorax valerianellae
Acinetobacter
Acinetobacter baumannii
Acinetobacter baylyi
Acinetobacter bouvetii
Acinetobacter calcoaceticus
Acinetobacter gerneri
Acinetobacter haemolyticus
Acinetobacter johnsonii
Acinetobacter junii
Acinetobacter lwoffi
Acinetobacter parvus
Acinetobacter radioresistens
Acinetobacter schindleri
Acinetobacter soli
Acinetobacter tandoii
Acinetobacter tjernbergiae
Acinetobacter towneri
Acinetobacter ursingii
Acinetobacter venetianus
Acrocarpospora
Acrocarpospora corrugata
Acrocarpospora macrocephala
Acrocarpospora pleiomorpha
Actibacter
Actibacter sediminis
Actinoalloteichus
Actinoalloteichus cyanogriseus
Actinoalloteichus hymeniacidonis
Actinoalloteichus spitiensis
Actinobaccillus
Actinobacillus capsulatus
Actinobacillus delphinicola
Actinobacillus hominis
Actinobacillus indolicus
Actinobacillus lignieresii
Actinobacillus minor
Actinobacillus muris
Actinobacillus pleuropneumoniae
Actinobacillus porcinus
Actinobacillus rossii
Actinobacillus scotiae
Actinobacillus seminis
Actinobacillus succinogenes
Actinobaccillus suis
Actinobacillus ureae
Actinobaculum
Actinobaculum massiliense
Actinobaculum schaalii
Actinobaculum suis
Actinomyces urinale
Actinocatenispora
Actinocatenispora rupis
Actinocatenispora thailandica
Actinocatenispora sera
Actinocorallia
Actinocorallia aurantiaca
Actinocorallia aurea
Actinocorallia cavernae
Actinocorallia glomerata
Actinocorallia herbida
Actinocorallia libanotica
Actinocorallia longicatena
Actinomadura
Actinomadura alba
Actinomadura atramentaria
Actinomadura bangladeshensis
Actinomadura catellatispora
Actinomadura chibensis
Actinomadura chokoriensis
Actinomadura citrea
Actinomadura coerulea
Actinomadura echinospora
Actinomadura fibrosa
Actinomadura formosensis
Actinomadura hibisca
Actinomadura kijaniata
Actinomadura latina
Actinomadura livida
Actinomadura luteofluorescens
Actinomadura macra
Actinomadura madurae
Actinomadura oligospora
Actinomadura pelletieri
Actinomadura rubrobrunea
Actinomadura rugatobispora
Actinomadura umbrina
Actinomadura verrucosospora
Actinomadura vinacea
Actinomadura viridilutea
Actinomadura viridis
Actinomadura yumaensis
Actinomyces
Actinomyces bovis
Actinomyces denticolens
Actinomyces europaeus
Actinomyces georgiae
Actinomyces gerencseriae
Actinomyces hordeovulneris
Actinomyces howellii
Actinomyces hyovaginalis
Actinomyces israelii
Actinomyces johnsonii
Actinomyces meyeri
Actinomyces naeslundii
Actinomyces neuii
Actinomyces odontolyticus
Actinomyces oris
Actinomyces radingae
Actinomyces slackii
Actinomyces turicensis
Actinomyces viscosus
Actinoplanes
Actinoplanes auranticolor
Actinoplanes brasiliensis
Actinoplanes consettensis
Actinoplanes deccanensis
Actinoplanes derwentensis
Actinoplanes digitatis
Actinoplanes durhamensis
Actinoplanes ferrugineus
Actinoplanes globisporus
Actinoplanes humidus
Actinoplanes italicus
Actinoplanes liguriensis
Actinoplanes lobatus
Actinoplanes missouriensis
Actinoplanes palleronii
Actinoplanes philippinensis
Actinoplanes rectilineatus
Actinoplanes regularis
Actinoplanes teichomyceticus
Actinoplanes utahensis
Actinopolyspora
Actinopolyspora halophila
Actinopolyspora mortivallis
Actinosynnema
Actinosynnema mirum
Actinotalea
Actinotalea fermentans
Aerococcus
Aerococcus sanguinicola
Aerococcus urinae
Aerococcus urinaeequi
Aerococcus urinaehominis
Aerococcus viridans
Aeromicrobium
Aeromicrobium erythreum
Aeromonas
Aeromonas allosaccharophila
Aeromonas bestiarum
Aeromonas caviae
Aeromonas encheleia
Aeromonas enteropelogenes
Aeromonas eucrenophila
Aeromonas ichthiosmia
Aeromonas jandaei
Aeromonas media
Aeromonas popoffii
Aeromonas sobria
Aeromonas veronii
Agrobacterium
Agrobacterium gelatinovorum
Agrococcus
Agrococcus citreus
Agrococcus jenensis
Agromonas
Agromonas oligotrophica
Agromyces
Agromyces fucosus
Agromyces hippuratus
Agromyces luteolus
Agromyces mediolanus
Agromyces ramosus
Agromyces rhizospherae
Akkermansia
Akkermansia muciniphila
Albidiferax
Albidiferax ferrireducens
Albidovulum
Albidovulum inexpectatum
Alcaligenes
Alcaligenes denitrificans
Alcaligenes faecalis
Alcanivorax
Alcanivorax borkumensis
Alcanivorax jadensis
Algicola
Algicola bacteriolytica
Alicyclobacillus
Alicyclobacillus disulfidooxidans
Alicyclobacillus sendaiensis
Alicyclobacillus vulcanalis
Alishewanella
Alishewanella fetalis
Alkalibacillus
Alkalibacillus haloalkaliphilus
Alkalilimnicola
Alkalilimnicola ehrlichii
Alkaliphilus
Alkaliphilus oremlandii
Alkaliphilus transvaalensis
Allochromatium
Allochromatium vinosum
Alloiococcus
Alloiococcus otitis
Allokutzneria
Allokutzneria albata
Altererythrobacter
Altererythrobacter ishigakiensis
Altermonas
Altermonas haloplanktis
Altermonas macleodii
Alysiella
Alysiella crassa
Alysiella filiformis
Aminobacter
Aminobacter aganoensis
Aminobacter aminovorans
Aminobacter niigataensis
Aminobacterium
Aminobacterium mobile
Aminomonas
Aminomonas paucivorans
Ammoniphilus
Ammoniphilus oxalaticus
Ammoniphilus oxalivorans
Amphibacillus
Amphibacillus xylanus
Amphritea
Amphritea balenae
Amphritea japonica
Amycolatopsis
Amycolatopsis alba
Amycolatopsis albidoflavus
Amycolatopsis azurea
Amycolatopsis coloradensis
Amycolatopsis lurida
Amycolatopsis mediterranei
Amycolatopsis rifamycinica
Amycolatopsis rubida
Amycolatopsis sulphurea
Amycolatopsis tolypomycina
Anabaena
Anabaena cylindrica
Anabaena flos-aquae
Anabaena variabilis
Anaeroarcus
Anaeroarcus burkinensis
Anaerobaculum
Anaerobaculum mobile
Anaerobiospirillum
Anaerobiospirillum succiniciproducens
Anaerobiospirillum thomasii
Anaerococcus
Anaerococcus hydrogenalis
Anaerococcus lactolyticus
Anaerococcus prevotii
Anaerococcus tetradius
Anaerococcus vaginalis
Anaerofustis
Anaerofustis stercorihominis
Anaeromusa
Anaeromusa acidaminophila
Anaeromyxobacter
Anaeromyxobacter dehalogenans
Anaerorhabdus
Anaerorhabdus furcosa
Anaerosinus
Anaerosinus glycerini
Anaerovirgula
Anaerovirgula multivorans
Ancalomicrobium
Ancalomicrobium adetum
Ancylobacter
Ancylobacter aquaticus
Aneurinibacillus
Aneurinibacillus aneurinilyticus
Aneurinibacillus migulanus
Aneurinibacillus thermoaerophilus
Angiococcus
Angiococcus disciformis
Angulomicrobium
Angulomicrobium tetraedrale
Anoxybacillus
Anoxybacillus pushchinoensis
Aquabacterium
Aquabacterium commune
Aquabacterium parvum
Aquaspirillum
Aquaspirillum polymorphum
Aquaspirillum putridiconchylium
Aquaspirillum serpens
Aquimarina
Aquimarina latercula
Arcanobacterium
Arcanobacterium haemolyticum
Arcanobacterium pyogenes
Archangium
Archangium gephyra
Arcobacter
Arcobacter butzleri
Arcobacter cryaerophilus
Arcobacter halophilus
Arcobacter nitrofigilis
Arcobacter skirrowii
Arhodomonas
Arhodomonas aquaeolei
Arsenophonus
Arsenophonus nasoniae
Arthrobacter
Arthrobacter agilis
Arthrobacter albus
Arthrobacter aurescens
Arthrobacter chlorophenolicus
Arthrobacter citreus
Arthrobacter crystallopoietes
Arthrobacter cumminsii
Arthrobacter globiformis
Arthrobacter histidinolovorans
Arthrobacter ilicis
Arthrobacter luteus
Arthrobacter methylotrophus
Arthrobacter mysorens
Arthrobacter nicotianae
Arthrobacter nicotinovorans
Arthrobacter oxydans
Arthrobacter pascens
Arthrobacter phenanthrenivorans
Arthrobacter polychromogenes
Atrhrobacter protophormiae
Arthrobacter psychrolactophilus
Arthrobacter ramosus
Arthrobacter sulfonivorans
Arthrobacter sulfureus
Arthrobacter uratoxydans
Arthrobacter ureafaciens
Arthrobacter viscosus
Arthrobacter woluwensis
Asaia
Asaia bogorensis
Asanoa
Asanoa ferruginea
Asticcacaulis
Asticcacaulis biprosthecium
Asticcacaulis excentricus
Atopobacter
Atopobacter phocae
Atopobium
Atopobium fossor
Atopobium minutum
Atopobium parvulum
Atopobium rimae
Atopobium vaginae
Aureobacterium
Aureobacterium barkeri
Aurobacterium
Aurobacterium liquefaciens
Avibacterium
Avibacterium avium
Avibacterium gallinarum
Avibacterium paragallinarum
Avibacterium volantium
Azoarcus
Azoarcus indigens
Azoarcus tolulyticus
Azoarcus toluvorans
Azohydromonas
Azohydromonas australica
Azohydromonas lata
Azomonas
Azomonas agilis
Azomonas insignis
Azomonas macrocytogenes
Azorhizobium
Azorhizobium caulinodans
Azorhizophilus
Azorhizophilus paspali
Azospirillum
Azospirillum brasilense
Azospirillum halopraeferens
Azospirillum irakense
Azotobacter
Azotobacter beijerinckii
Azotobacter chroococcum
Azotobacter nigricans
Azotobacter salinestris
Azotobacter vinelandii
Bacillus
[see below]
Bacteriovorax
Bacteriovorax stolpii
Bacteroides
Bacteroides caccae
Bacteroides coagulans
Bacteroides eggerthii
Bacteroides fragilis
Bacteroides galacturonicus
Bacteroides helcogenes
Bacteroides ovatus
Bacteroides pectinophilus
Bacteroides pyogenes
Bacteroides salyersiae
Bacteroides stercoris
Bacteroides suis
Bacteroides tectus
Bacteroides thetaiotaomicron
Bacteroides uniformis
Bacteroides ureolyticus
Bacteroides vulgatus
Balnearium
Balnearium lithotrophicum
Balneatrix
Balneatrix alpica
Balneola
Balneola vulgaris
Barnesiella
Barnesiella viscericola
Bartonella
Bartonella alsatica
Bartonella bacilliformis
Bartonella clarridgeiae
Bartonella doshiae
Bartonella elizabethae
Bartonella grahamii
Bartonella henselae
Bartonella rochalimae
Bartonella vinsonii
Bavariicoccus
Bavariicoccus seileri
Bdellovibrio
Bdellovibrio bacteriovorus
Bdellovibrio exovorus
Beggiatoa
Beggiatoa alba
Beijerinckia
Beijerinckia derxii
Beijerinckia fluminensis
Beijerinckia indica
Beijerinckia mobilis
Belliella
Belliella baltica
Bellilinea
Bellilinea caldifistulae
Belnapia
Belnapia moabensis
Bergeriella
Bergeriella denitrificans
Beutenbergia
Beutenbergia cavernae
Bibersteinia
Bibersteinia trehalosi
Bifidobacterium
Bifidobacterium adolescentis
Bifidobacterium angulatum
Bifidobacterium animalis
Bifidobacterium asteroides
Bifidobacterium bifidum
Bifidobacterium boum
Bifidobacterium breve
Bifidobacterium catenulatum
Bifidobacterium choerinum
Bifidobacterium coryneforme
Bifidobacterium cuniculi
Bifidobacterium dentium
Bifidobacterium gallicum
Bifidobacterium gallinarum
Bifidobacterium indicum
Bifidobacterium longum
Bifidobacterium magnum
Bifidobacterium merycicum
Bifidobacterium minimum
Bifidobacterium pseudocatenulatum
Bifidobacterium pseudolongum
Bifidobacterium pullorum
Bifidobacterium ruminantium
Bifidobacterium saeculare
Bifidobacterium subtile
Bifidobacterium thermophilum
Bilophila
Bilophila wadsworthia
Biostraticola
Biostraticola tofi
Bizionia
Bizionia argentinensis
Blastobacter
Blastobacter capsulatus
Blastobacter denitrificans
Blastococcus
Blastococcus aggregatus
Blastococcus saxobsidens
Blastochloris
Blastochloris viridis
Blastomonas
Blastomonas natatoria
Blastopirellula
Blastopirellula marina
Blautia
Blautia coccoides
Blautia hansenii
Blautia producta
Blautia wexlerae
Bogoriella
Bogoriella caseilytica
Bordetella
Bordetella avium
Bordetella bronchiseptica
Bordetella hinzii
Bordetella holmesii
Bordetella parapertussis
Bordetella pertussis
Bordetella petrii
Bordetella trematum
Borrelia
Borrelia afzelii
Borrelia americana
Borrelia burgdorferi
Borrelia carolinensis
Borrelia coriaceae
Borrelia garinii
Borrelia japonica
Bosea
Bosea minatitlanensis
Bosea thiooxidans
Brachybacterium
Brachybacierium alimentarium
Brachybacterium faecium
Brachybacterium paraconglomeratum
Brachybacterium rhamnosum
Brachybacterium tyrofermentans
Brachyspira
Brachyspira alvinipulli
Brachyspira hyodysenteriae
Brachyspira innocens
Brachyspira murdochii
Brachyspira pilosicoli
Bradyrhizobium
Bradyrhizobium canariense
Bradyrhizobium elkanii
Bradyrhizobium japonicum
Bradyrhizobium liaoningense
Brenneria
Brenneria alni
Brenneria nigrifluens
Brenneria quercina
Brenneria quercina
Brenneria salicis
Brevibacillus
Brevibacillus agri
Brevibacillus borstelensis
Brevibacillus brevis
Brevibacillus centrosporus
Brevibacillus choshinensis
Brevibacillus invocatus
Brevibacillus laterosporus
Brevibacillus parabrevis
Brevibacillus reuszeri
Brevibacterium
Brevibacterium abidum
Brevibacterium album
Brevibacterium aurantiacum
Brevibacterium celere
Brevibacterium epidermidis
Brevibacterium frigoritolerans
Brevibacterium halotolerans
Brevibacterium iodinum
Brevibacterium linens
Brevibacterium lyticum
Brevibacterium mcbrellneri
Brevibacterium otitidis
Brevibacterium oxydans
Brevibacterium paucivorans
Brevibacterium stationis
Brevinema
Brevinema andersonii
Brevundimonas
Brevundimonas alba
Brevundimonas aurantiaca
Brevundimonas diminuta
Brevundimonas intermedia
Brevundimonas subvibrioides
Brevundimonas vancanneytii
Brevundimonas variabilis
Brevundimonas vesicularis
Brochothrix
Brochothrix campestris
Brochothrix thermosphacta
Brucella
Brucella canis
Brucella neotomae
Bryobacter
Bryobacter aggregatus
Burkholderia
Burkholderia ambifaria
Burkholderia andropogonis
Burkholderia anthina
Burkholderia caledonica
Burkholderia caryophylli
Burkholderia cenocepacia
Burkholderia cepacia
Burkholderia cocovenenans
Burkholderia dolosa
Burkholderia fungorum
Burkholderia glathei
Burkholderia glumae
Burkholderia graminis
Burkholderia kururiensis
Burkholderia multivorans
Burkholderia phenazinium
Burkholderia plantarii
Burkholderia pyrrocinia
Burkholderia silvatlantica
Burkholderia stabilis
Burkholderia thailandensis
Burkholderia tropica
Burkholderia unamae
Burkholderia vietnamiensis
Buttiauxella
Buttiauxella agrestis
Buttiauxella brennerae
Buttiauxella ferragutiae
Buttiauxella gaviniae
Buttiauxella izardii
Buttiauxella noackiae
Buttiauxella warmboldiae
Butyrivibrio
Butyrivibrio fibrisolvens
Butyrivibrio hungatei
Butyrivibrio proteoclasticus
Bacillus
B. acidiceler
B. acidicola
B. acidiproducens
B. acidocaldarius
B. acidoterrestris
B. aeolius
B. aerius
B. aerophilus
B. agaradhaerens
B. agri
B. aidingensis
B. akibai
B. alcalophilus
B. algicola
B. alginolyticus
B. alkalidiazotrophicus
B. alkalinitrilicus
B. alkalisediminis
B. alkalitelluris
B. altitudinis
B. alveayuensis
B. alvei
B. amyloliquefaciens
B.a. subsp. amyloliquefaciens
B.a. subsp. plantarum
B. dipsosauri
B. drentensis
B. edaphicus
B. ehimensis
B. eiseniae
B. enclensis
B. endophyticus
B. endoradicis
B. farraginis
B. fastidiosus
B. fengqiuensis
B. firmus
B. flexus
B. foraminis
B. fordii
B. formosus
B. fortis
B. fumarioli
B. funiculus
B. fusiformis
B. galactophilus
B. galactosidilyticus
B. galliciensis
B. gelatini
B. gibsonii
B. ginsengi
B. ginsengihumi
B. ginsengisoli
B. globisporus
(eg, B.g. subsp. Globisporus; or
B.g. subsp. Marinus)
B. aminovorans
B. amylolyticus
B. andreesenii
B. aneurinilyticus
B. anthracis
B. aquimaris
B. arenosi
B. arseniciselenatis
B. arsenicus
B. aurantiacus
B. arvi
B. aryabhattai
B. asahii
B. atrophaeus
B. axarquiensis
B. azotofixans
B. azotoformans
B. badius
B. barbaricus
B. bataviensis
B. beijingensis
B. benzoevorans
B. beringensis
B. berkeleyi
B. beveridgei
B. bogoriensis
B. boroniphilus
B. borstelensis
B. brevis Migula
B. butanolivorans
B. canaveralius
B. carboniphilus
B. cecembensis
B. cellulosilyticus
B. centrosporus
B. cereus
B. chagannorensis
B. chitinolyticus
B. chondroitinus
B. choshinensis
B. chungangensis
B. cibi
B. circulans
B. clarkii
B. clausii
B. coagulans
B. coahuilensis
B. cohnii
B. composti
B. curdlanolyticus
B. cycloheptanicus
B. cytotoxicus
B. daliensis
B. decisifrondis
B. decolorationis
B. deserti
B. glucanolyticus
B. gordonae
B. gottheilii
B. graminis
B. halmapalus
B. haloalkaliphilus
B. halochares
B. halodenitrificans
B. halodurans
B. halophilus
B. halosaccharovorans
B. hemicellulosilyticus
B. hemicentroti
B. herbersteinensis
B. horikoshii
B. horneckiae
B. horti
B. huizhouensis
B. humi
B. hwajinpoensis
B. idriensis
B. indicus
B. infantis
B. infernus
B. insolitus
B. invictae
B. iranensis
B. isabeliae
B. isronensis
B. jeotgali
B. kaustophilus
B. kobensis
B. kochii
B. kokeshiiformis
B. koreensis
B. korlensis
B. kribbensis
B. krulwichiae
B. laevolacticus
B. larvae
B. laterosporus
B. salexigens
B. saliphilus
B. schlegelii
B. sediminis
B. selenatarsenatis
B. selenitireducens
B. seohaeanensis
B. shacheensis
B. shackletonii
B. siamensis
B. silvestris
B. simplex
B. siralis
B. smithii
B. soli
B. solimangrovi
B. solisalsi
B. songklensis
B. sonorensis
B. sphaericus
B. sporothermodurans
B. stearothermophilus
B. stratosphericus
B. subterraneus
B. subtilis
(eg, B.s. subsp. Inaquosorum; or
B.s. subsp. Spizizeni; or
B.s. subsp. Subtilis)
B. taeanensis
B. tequilensis
B. thermantarcticus
B. thermoaerophilus
B. thermoamylovorans
B. thermocatenulatus
B. thermocloacae
B. thermocopriae
B. thermodenitrificans
B. thermoglucosidasius
B. thermolactis
B. thermoleovorans
B. thermophilus
B. thermoruber
B. thermosphaericus
B. thiaminolyticus
B. thioparans
B. thuringiensis
B. tianshenii
B. trypoxylicola
B. tusciae
B. validus
B. vallismortis
B. vedderi
B. velezensis
B. vietnamensis
B. vireti
B. vulcani
B. wakoensis
B. weihenstephanensis
B. xiamenensis
B. xiaoxiensis
B. zhanjiangensis
B. peoriae
B. persepolensis
B. persicus
B. pervagus
B. plakortidis
B. pocheonensis
B. polygoni
B. polymyxa
B. popilliae
B. pseudalcalophilus
B. pseudofirmus
B. pseudomycoides
B. psychrodurans
B. psychrophilus
B. psychrosaccharolyticus
B. psychrotolerans
B. pulvifaciens
B. pumilus
B. purgationiresistens
B. pycnus
B. qingdaonensis
B. qingshengii
B. reuszeri
B. rhizosphaerae
B. rigui
B. ruris
B. safensis
B. salarius
B. lautus
B. lehensis
B. lentimorbus
B. lentus
B. licheniformis
B. ligniniphilus
B. litoralis
B. locisalis
B. luciferensis
B. luteolus
B. luteus
B. macauensis
B. macerans
B. macquariensis
B. macyae
B. malacitensis
B. mannanilyticus
B. marisflavi
B. marismortui
B. marmarensis
B. massiliensis
B. megaterium
B. mesonae
B. methanolicus
B. methylotrophicus
B. migulanus
B. mojavensis
B. mucilaginosus
B. muralis
B. murimartini
B. mycoides
B. naganoensis
B. nanhaiensis
B. nanhaiisediminis
B. nealsonii
B. neidei
B. neizhouensis
B. niabensis
B. niacini
B. novalis
B. oceanisediminis
B. odysseyi
B. okhensis
B. okuhidensis
B. oleronius
B. oryzaecorticis
B. oshimensis
B. pabuli
B. pakistanensis
B. pallidus
B. pallidus
B. panacisoli
B. panaciterrae
B. pantothenticus
B. parabrevis
B. paraflexus
B. pasteurii
B. patagoniensis
Caenimonas
Caenimonas koreensis
Caldalkalibacillus
Caldalkalibacillus uzonensis
Caldanaerobacter
Caldanaerobacter subterraneus
Caldanaerobius
Caldanaerobius fijiensis
Caldanaerobius polysaccharolyticus
Caldanaerobius zeae
Caldanaerovirga
Caldanaerovirga acetigignens
Caldicellulosiruptor
Caldicellulosiruptor bescii
Caldicellulosiruptor kristjanssonii
Caldicellulosiruptor owensensis
Campylobacter
Campylobacter coli
Campylobacter concisus
Campylobacter curvus
Campylobacter fetus
Campylobacter gracilis
Campylobacter helveticus
Campylobacter hominis
Campylobacter hyointestinalis
Campylobacter jejuni
Campylobacter lari
Campylobacter mucosalis
Campylobacter rectus
Campylobacter showae
Campylobacter sputorum
Campylobacter upsaliensis
Capnocytophaga
Capnocytophaga canimorsus
Capnocytophaga cynodegmi
Capnocytophaga gingivalis
Capnocytophaga granulosa
Capnocytophaga haemolytica
Capnocytophaga ochracea
Capnocytophaga sputigena
Cardiobacterium
Cardiobacterium hominis
Carnimonas
Carnimonas nigrificans
Carnobacterium
Carnobacterium alterfunditum
Carnobacterium divergens
Carnobacterium funditum
Carnobacterium gallinarum
Carnobacterium maltaromaticum
Carnobacterium mobile
Carnobacterium viridans
Caryophanon
Caryophanon latum
Caryophanon tenue
Catellatospora
Catellatospora citrea
Catellatospora methionotrophica
Catenococcus
Catenococcus thiocycli
Catenuloplanes
Catenuloplanes atrovinosus
Catenuloplanes castaneus
Catenuloplanes crispus
Catenuloplanes indicus
Catenuloplanes japonicus
Catenuloplanes nepalensis
Catenuloplanes niger
Chryseobacterium
Chryseobacterium balustinum
Citrobacter
C. amalonaticus
C. braakii
C. diversus
C. farmeri
C. freundii
C. gillenii
C. koseri
C. murliniae
C. pasteurii^{[1]}
C. rodentium
C. sedlakii
C. werkmanii
C. youngae
Clostridium
(see below)
Coccochloris
Coccochloris elabens
Corynebacterium
Corynebacterium flavescens
Corynebacterium variabile
Curtobacterium
Curtobacterium albidum
Curtobacterium citreus
Clostridium
Clostridium absonum,
Clostridium aceticum,
Clostridium acetireducens,
Clostridium acetobutylicum,
Clostridium acidisoli,
Clostridium aciditolerans,
Clostridium acidurici,
Clostridium aerotolerans,
Clostridium aestuarii,
Clostridium akagii,
Clostridium aldenense,
Clostridium aldrichii,
Clostridium algidicarni,
Clostridium algidixylanolyticum,
Clostridium algifaecis,
Clostridium algoriphilum,
Clostridium alkalicellulosi,
Clostridium aminophilum,
Clostridium aminovalericum,
Clostridium amygdalinum,
Clostridium amylolyticum,
Clostridium arbusti,
Clostridium arcticum,
Clostridium argentinense,
Clostridium asparagiforme,
Clostridium aurantibutyricum,
Clostridium autoethanogenum,
Clostridium baratii,
Clostridium barkeri,
Clostridium bartlettii,
Clostridium beijerinckii,
Clostridium bifermentans,
Clostridium bolteae,
Clostridium bornimense,
Clostridium botulinum,
Clostridium bowmanii,
Clostridium bryantii,
Clostridium butyricum,
Clostridium cadaveris,
Clostridium caenicola,
Clostridium caminithermale,
Clostridium carboxidivorans,
Clostridium carnis,
Clostridium cavendishii,
Clostridium celatum,
Clostridium celerecrescens,
Clostridium cellobioparum,
Clostridium cellulofermentans,
Clostridium cellulolyticum,
Clostridium cellulosi,
Clostridium cellulovorans,
Clostridium chartatabidum,
Clostridium chouvoei,
Clostridium chromiireducens,
Clostridium citroniae,
Clostridium clariflavum,
Clostridium clostridioforme,
Clostridium coccoides,
Clostridium cochlearium,
Clostridium colletant,
Clostridium colicanis,
Clostridium colinum,
Clostridium collagenovorans,
Clostridium cylindrosporum,
Clostridium difficile,
Clostridium diolis,
Clostridium disporicum,
Clostridium drakei,
Clostridium durum,
Clostridium estertheticum,
Clostridium estertheticum estertheticum,
Clostridium estertheticum laramiense,
Clostridium fallax,
Clostridium felsineum,
Clostridium fervidum,
Clostridium fimetarium,
Clostridium formicaceticum,
Clostridium frigidicarnis,
Clostridium frigoris,
Clostridium ganghwense,
Clostridium gasigenes,
Clostridium ghonii,
Clostridium glycolicum,
Clostridium glycyrrhizinilyticum,
Clostridium grantii,
Clostridium haemolyticum,
Clostridium halophilum,
Clostridium hastiforme,
Clostridium hathewayi,
Clostridium herbivorans,
Clostridium hiranonis,
Clostridium histolyticum,
Clostridium homopropionicum,
Clostridium huakuii,
Clostridium hungatei,
Clostridium hydrogeniformans,
Clostridium hydroxybenzoicum,
Clostridium hylemonae,
Clostridium jejuense,
Clostridium indolis,
Clostridium innocuum,
Clostridium intestinale,
Clostridium irregulare,
Clostridium isatidis,
Clostridium josui,
Clostridium kluyveri,
Clostridium lactatifermentans,
Clostridium lacusfryxellense,
Clostridium laramiense,
Clostridium lavalense,
Clostridium lentocellum,
Clostridium lentoputrescens,
Clostridium leptum,
Clostridium limosum,
Clostridium litorale,
Clostridium lituseburense,
Clostridium ljungdahlii,
Clostridium lortetii,
Clostridium lundense,
Clostridium magnum,
Clostridium malenominatum,
Clostridium mangenotii,
Clostridium mayombei,
Clostridium methoxybenzovorans,
Clostridium methylpentosum,
Clostridium neopropionicum,
Clostridium nexile,
Clostridium nitrophenolicum,
Clostridium novyi,
Clostridium oceanicum,
Clostridium orbiscindens,
Clostridium oroticum,
Clostridium oxalicum,
Clostridium papyrosolvens,
Clostridium paradoxum,
Clostridium paraperfringens
(Alias: C. welchii),
Clostridium paraputrificum,
Clostridium pascui,
Clostridium pasteurianum,
Clostridium peptidivorans,
Clostridium perenne,
Clostridium perfringens,
Clostridium pfennigii,
Clostridium phytofermentans,
Clostridium piliforme,
Clostridium polysaccharolyticum,
Clostridium populeti,
Clostridium propionicum,
Clostridium proteoclasticum,
Clostridium proteolyticum,
Clostridium psychrophilum,
Clostridium puniceum,
Clostridium purinilyticum,
Clostridium putrefaciens,
Clostridium putrificum,
Clostridium quercicolum,
Clostridium quinii,
Clostridium ramosum,
Clostridium rectum,
Clostridium roseum,
Clostridium saccharobutylicum,
Clostridium saccharogumia,
Clostridium saccharolyticum,
Clostridium saccharoperbutylacetonicum,
Clostridium sardiniense,
Clostridium sartagoforme,
Clostridium scatologenes,
Clostridium schirmacherense,
Clostridium scindens,
Clostridium septicum,
Clostridium sordellii,
Clostridium sphenoides,
Clostridium spiroforme,
Clostridium sporogenes,
Clostridium sporosphaeroides,
Clostridium stercorarium,
Clostridium stercorarium leptospartum,
Clostridium stercorarium stercorarium,
Clostridium stercorarium thermolacticum,
Clostridium sticklandii,
Clostridium straminisolvens,
Clostridium subterminale,
Clostridium sufflavum,
Clostridium sulfidigenes,
Clostridium symbiosum,
Clostridium tagluense,
Clostridium tepidiprofundi,
Clostridium termitidis,
Clostridium tertium,
Clostridium tetani,
Clostridium tetanomorphum,
Clostridium thermaceticum,
Clostridium thermautotrophicum,
Clostridium thermoalcaliphilum,
Clostridium thermobutyricum,
Clostridium thermocellum,
Clostridium thermocopriae,
Clostridium thermohydrosulfuricum,
Clostridium thermolacticum,
Clostridium thermopalmarium,
Clostridium thermopapyrolyticum,
Clostridium thermosaccharolyticum,
Clostridium thermosuccinogenes,
Clostridium thermosulfurigenes,
Clostridium thiosulfatireducens,
Clostridium tyrobutyricum,
Clostridium uliginosum,
Clostridium ultunense,
Clostridium villosum,
Clostridium vincentii,
Clostridium viride,
Clostridium xylanolyticum,
Clostridium xylanovorans
Dactylosporangium
Dactylosporangium aurantiacum
Dactylosporangium fulvum
Dactylosporangium matsuzakiense
Dactylosporangium roseum
Dactylosporangium thailandense
Dactylosporangium vinaceum
Deinococcus
Deinococcus aerius
Deinococcus apachensis
Deinococcus aquaticus
Deinococcus aquatilis
Deinococcus caeni
Deinococcus radiodurans
Deinococcus radiophilus
Delftia
Delftia acidovorans
Desulfovibrio
Desulfovibrio desulfuricans
Diplococcus
Diplococcus pneumoniae
Echinicola
Echinicola pacifica
Echinicola vietnamensis
Enterobacter
E. aerogenes
E. amnigenus
E. agglomerans
E. arachidis
E. asburiae
E. cancerogenous
E. cloacae
E. cowanii
E. dissolvens
E. gergoviae
E. helveticus
E. hormaechei
E. intermedius
Enterobacter kobei
E. ludwigii
E. mori
E. nimipressuralis
E. oryzae
E. pulveris
E. pyrinus
E. radicincitans
E. taylorae
E. turicensis
E. sakazakii
Enterobacter soli
Enterococcus
Enterococcus durans
Enterococcus faecalis
Enterococcus faecium
Erwinia
Erwinia hapontici
Escherichia
Escherichia coli
Faecalibacterium
Faecalibacterium prausnitzii
Fangia
Fangia hongkongensis
Fastidiosipila
Fastidiosipila sanguinis
Fusobacterium
Fusobacterium nucleatum
Flavobacterium
Flavobacterium antarcticum
Flavobacterium aquatile
Flavobacterium aquidurense
Flavobacterium balustinum
Flavobacterium croceum
Flavobacterium cucumis
Flavobacterium daejeonense
Flavobacterium defluvii
Flavobacterium degerlachei
Flavobacterium denitrificans
Flavobacterium filum
Flavobacterium flevense
Flavobacterium frigidarium
Flavobacterium mizutaii
Flavobacterium okeanokoites
Gaetbulibacter
Gaetbulibacter saemankumensis
Gallibacterium
Gallibacterium anatis
Gallicola
Gallicola barnesae
Garciella
Garciella nitratireducens
Geobacillus
Geobacillus thermoglucosidasius
Geobacillus stearothermophilus
Geobacter
Geobacter bemidjiensis
Geobacter bremensis
Geobacter chapellei
Geobacter grbiciae
Geobacter hydrogenophilus
Geobacter lovleyi
Geobacter metallireducens
Geobacter pelophilus
Geobacter pickeringii
Geobacter sulfurreducens
Geodermatophilus
Geodermatophilus obscurus
Gluconacetobacter
Gluconacetobacter xylinus
Gordonia
Gordonia rubripertincta
Haemophilus
Haemophilus aegyptius
Haemophilus aphrophilus
Haemophilus felis
Haemophilus gallinarum
Haemophilus haemolyticus
Haemophilus influenzae
Haemophilus paracuniculus
Haemophilus parahaemolyticus
Haemophilus parainfluenzae
Haemophilus paraphrohaemolyticus
Haemophilus parasuis
Haemophilus pittmaniae
Hafnia
Hafnia alvei
Hahella
Hahella ganghwensis
Halalkalibacillus
Halalkalibacillus halophilus
Helicobacter
Helicobacter pylori
Ideonella
Ideonella azotifigens
Idiomarina
Idiomarina abyssalis
Idiomarina baltica
Idiomarina fontislapidosi
Idiomarina loihiensis
Idiomarina ramblicola
Idiomarina seosinensis
Idiomarina zobellii
Ignatzschineria
Ignatzschineria larvae
Ignavigranum
Ignavigranum ruoffiae
Ilumatobacter
Ilumatobacter fluminis
Ilyobacter
Ilyobacter delafieldii
Ilyobacter insuetus
Ilyobacter polytropus
Ilyobacter tartaricus
Janibacter
Janibacter anophelis
Janibacter corallicola
Janibacter limosus
Janibacter melonis
Janibacter terrae
Jannaschia
Jannaschia cystaugens
Jannaschia helgolandensis
Jannaschia pohangensis
Jannaschia rubra
Janthinobacterium
Janthinobacterium agaricidamnosum
Janthinobacterium lividum
Jejuia
Jejuia pallidilutea
Jeotgalibacillus
Jeotgalibacillus alimentarius
Jeotgalicoccus
Jeotgalicoccus halotolerans
Kaistia
Kaistia adipata
Kaistia soli
Kangiella
Kangiella aquimarina
Kangiella koreensis
Kerstersia
Kerstersia gyiorum
Kiloniella
Kiloniella laminariae
Klebsiella
K. granulomatis
K. oxytoca
K. pneumoniae
K. terrigena
K. variicola
Kluyvera
Kluyvera ascorbata
Kocuria
Kocuria roasea
Kocuria varians
Kurthia
Kurthia zopfii
Labedella
Labedella gwakjiensis
Labrenzia
Labrenzia aggregata
Labrenzia alba
Labrenzia alexandrii
Labrenzia marina
Labrys
Labrys methylaminiphilus
Labrys miyagiensis
Labrys monachus
Labrys okinawensis
Labrys portucalensis
Lactobacillus
[see below]
Laceyella
Laceyella putida
Lechevalieria
Lechevalieria aerocolonigenes
Legionella
[see below]
Listeria
L. aquatica
L. booriae
L. cornellensis
L. fleischmannii
L. floridensis
L. grandensis
L. grayi
L. innocua
Listeria ivanovii
L. marthii
L. monocytogenes
L. newyorkensis
L. riparia
L. rocourtiae
L. seeligeri
L. weihenstephanensis
L. welshimeri
Listonella
Listonella anguillarum
Macrococcus
Macrococcus bovicus
Marinobacter
Marinobacter algicola
Marinobacter bryozoorum
Marinobacter flavimaris
Meiothermus
Meiothermus ruber
Methylophilus
Methylophilus methylotrophus
Microbacterium
Microbacterium ammoniaphilum
Microbacterium arborescens
Microbacterium liquefaciens
Microbacterium oxydans
Micrococcus
Micrococcus luteus
Micrococcus lylae
Moraxella
Moraxella bovis
Moraxella nonliquefaciens
Moraxella osloensis
Nakamurella
Nakamurella multipartita
Nannocystis
Nannocystis pusilla
Natranaerobius
Natranaerobius thermophilus
Natranaerobius trueperi
Naxibacter
Naxibacter alkalitolerans
Neisseria
Neisseria cinerea
Neisseria denitrificans
Neisseria gonorrhoeae
Neisseria lactamica
Neisseria mucosa
Neisseria sicca
Neisseria subflava
Neptunomonas
Neptunomonas japonica
Nesterenkonia
Nesterenkonia holobia
Nocardia
Nocardia argentinensis
Nocardia corallina
Nocardia otitidiscaviarum
Lactobacillus
L. acetotolerans
L. acidifarinae
L. acidipiscis
L. acidophilus
Lactobacillus agilis
L. algidus
L. alimentarius
L. amylolyticus
L. amylophilus
L. amylotrophicus
L. amylovorus
L. animalis
L. antri
L. apodemi
L. aviarius
L. bifermentans
L. brevis
L. buchneri
L. camelliae
L. casei
L. kitasatonis
L. kunkeei
L. leichmannii
L. lindneri
L. malefermentans
L. catenaformis
L. ceti
L. coleohominis
L. collinoides
L. composti
L. concavus
L. coryniformis
L. crispatus
L. crustorum
L. curvatus
L. delbrueckii subsp. bulgaricus
L. delbrueckii subsp. delbrueckii
L. delbrueckii subsp. lactis
L. dextrinicus
L. diolivorans
L. equi
L. equigenerosi
L. farraginis
L. farciminis
L. fermentum
L. fornicalis
L. fructivorans
L. frumenti
L. mali
L. manihotivorans
L. mindensis
L. mucosae
L. murinus
L. nagelii
L. namurensis
L. nantensis
L. oligofermentans
L. oris
L. panis
L. pantheris
L. parabrevis
L. parabuchneri
L. paracasei
L. paracollinoides
L. parafarraginis
L. homohiochii
L. iners
L. ingluviei
L. intestinalis
L. fuchuensis
L. gallinarum
L. gasseri
L. parakefiri
L. paralimentarius
L. paraplantarum
L. pentosus
L. perolens
L. plantarum
L. pontis
L. protectus
L. psittaci
L. rennini
L. reuteri
L. rhamnosus
L. rimae
L. rogosae
L. rossiae
L. ruminis
L. saerimneri
L. jensenii
L. johnsonii
L. kalixensis
L. kefiranofaciens
L. kefiri
L. kimchii
L. helveticus
L. hilgardii
L. sakei
L. salivarius
L. sanfranciscensis
L. satsumemis
L. secaliphilus
L. sharpeae
L. siliginis
L. spicheri
L. suebicus
L. thailandensis
L. ultunensis
L. vaccinostercus
L. vaginalis
L. versmoldensis
L. vini
L. vitulinus
L. zeae
L. zymae
L. gastricus
L. ghanensis
L. graminis
L. hammesii
L. hamsteri
L. harbinensis
L. hayakitensis
Legionella
Legionella adelaidensis
Legionella anisa
Legionella beliardensis
Legionella birminghamensis
Legionella bozemanae
Legionella brunensis
Legionella busanensis
Legionella cardiaca
Legionella cherrii
Legionella cincinnatiensis
Legionella clemsonensis
Legionella donaldsonii
Legionella drancourtii
Legionella dresdenensis
Legionella drozanskii
Legionella dumoffii
Legionella erythra
Legionella fairfieldensis
Legionella fallonii
Legionella feeleii
Legionella geestiana
Legionella genomospecies
Legionella gormanii
Legionella gratiana
Legionella gresilensis
Legionella hackeliae
Legionella impletisoli
Legionella israelensis
Legionella jamestowniensis
Candidatus Legionella jeonii
Legionella jordanis
Legionella lansingensis
Legionella londiniensis
Legionella longbeachae
Legionella lytica
Legionella maceachernii
Legionella massiliensis
Legionella micdadei
Legionella monrovica
Legionella moravica
Legionella nagasakiensis
Legionella nautarum
Legionella norrlandica
Legionella oakridgensis
Legionella parisiensis
Legionella pittsburghensis
Legionella pneumophila
Legionella quateirensis
Legionella quinlivanii
Legionella rowbothamii
Legionella rubrilucens
Legionella sainthelensi
Legionella santicrucis
Legionella shakespearei
Legionella spiritensis
Legionella steelei
Legionella steigerwaltii
Legionella taurinensis
Legionella tucsonensis
Legionella tunisiensis
Legionella wadsworthii
Legionella waltersii
Legionella worsleiensis
Legionella yabuuchiae
Oceanibulbus
Oceanibulbus indolifex
Oceanicaulis
Oceanicaulis alexandrii
Oceanicola
Oceanicola batsensis
Oceanicola granulosus
Oceanicola nanhaiensis
Oceanimonas
Oceanimonas baumannii
Oceaniserpentilla
Oceaniserpentilla haliotis
Oceanisphaera
Oceanisphaera donghaensis
Oceanisphaera litoralis
Oceanithermus
Oceanithermus desulfurans
Oceanithermus profundus
Oceanobacillus
Oceanobacillus caeni
Oceanospirillum
Oceanospirillum linum
Paenibacillus
Paenibacillus thiaminolyticus
Pantoea
Pantoea agglomerans
Paracoccus
Paracoccus alcaliphilus
Paucimonas
Paucimonas lemoignei
Pectobacterium
Pectobacterium aroidearum
Pectobacterium atrosepticum
Pectobacterium betavasculorum
Pectobacterium cacticida
Pectobacterium carnegieana
Pectobacterium carotovorum
Pectobacterium chrysanthemi
Pectobacterium cypripedii
Pectobacterium rhapontici
Pectobacterium wasabiae
Planococcus
Planococcus citreus
Planomicrobium
Planomicrobium okeanokoites
Plesiomonas
Plesiomonas shigelloides
Proteus
Proteus vulgaris
Prevotella
Prevotella albensis
Prevotella amnii
Prevotella bergensis
Prevotella bivia
Prevotella brevis
Prevotella bryantii
Prevotella buccae
Prevotella buccalis
Prevotella copri
Prevotella dentalis
Prevotella denticola
Prevotella disiens
Prevotella histicola
Prevotella intermedia
Prevotella maculosa
Prevotella marshii
Prevotella melaninogenica
Prevotella micans
Prevotella multiformis
Prevotella nigrescens
Prevotella oralis
Prevotella oris
Prevotella oulorum
Prevotella pallens
Prevotella salivae
Prevotella stercorea
Prevotella tannerae
Prevotella timonensis
Prevotella veroralis
Providencia
Providencia stuartii
Pseudomonas
Pseudomonas aeruginosa
Pseudomonas alcaligenes
Pseudomonas anguillispetica
Pseudomonas fluorescens
Pseudoalteromonas haloplanktis
Pseudomonas mendocina
Pseudomonas pseudoalcaligenes
Pseudomonas putida
Pseudomonas tutzeri
Pseudomonas syringae
Psychrobacter
Psychrobacter faecalis
Psychrobacter phenylpyruvicus
Quadrisphaera
Quadrisphaera granulorum
Quatrionicoccus
Quatrionicoccus australiensis
Quinella
Quinella ovalis
Ralstonia
Ralstonia eutropha
Ralstonia insidiosa
Ralstonia mannitolilytica
Ralstonia pickettii
Ralstonia pseudosolanacearum
Ralstonia syzygii
Ralstonia solanacearum
Ramlibacter
Ramlibacter henchirensis
Ramlibacter tataouinensis
Raoultella
Raoultella ornithinolytica
Raoultella planticola
Raoultella terrigena
Rathayibacter
Rathayibacter caricis
Rathayibacter festucae
Rathayibacter iranicus
Rathayibacter rathayi
Rathayibacter toxicus
Rathayibacter tritici
Rhodobacter
Rhodobacter sphaeroides
Ruegeria
Ruegeria gelatinovorans
Saccharococcus
Saccharococcus thermophilus
Saccharomonospora
Saccharomonospora azurea
Saccharomonospora cyanea
Saccharomonospora viridis
Saccharophagus
Saccharophagus degradans
Saccharopolyspora
Saccharopolyspora erythraea
Saccharopolyspora gregorii
Saccharopolyspora hirsuta
Saccharopolyspora hordei
Saccharopolyspora rectivirgula
Saccharopolyspora spinosa
Saccharopolyspora taberi
Saccharothrix
Saccharothrix australiensis
Saccharothrix coeruleofusca
Saccharothrix espanaensis
Saccharothrix longispora
Saccharothrix mutabilis
Saccharothrix syringae
Saccharothrix tangerinus
Saccharothrix texasensis
Sagittula
Sagittula stellata
Salegentibacter
Salegentibacter salegens
Salimicrobium
Salimicrobium album
Salinibacter
Salinibacter ruber
Salinicoccus
Salinicoccus alkaliphilus
Salinicoccus hispanicus
Salinicoccus roseus
Salinispora
Salinispora arenicola
Salinispora tropica
Salinivibrio
Salinivibrio costicola
Salmonella
Salmonella bongori
Salmonella enterica
Salmonella subterranea
Salmonella typhi
Sanguibacter
Sanguibacter keddieii
Sanguibacter suarezii
Saprospira
Saprospira grandis
Sarcina
Sarcina maxima
Sarcina ventriculi
Sebaldella
Sebaldella termitidis
Serratia
Serratia fonticola
Serratia marcescens
Sphaerotilus
Sphaerotilus natans
Sphingobacterium
Sphingobacterium multivorum
Staphylococcus
[see below]
Stenotrophomonas
Stenotrophomonas maltophilia
Streptococcus
[also see below]
Streptomyces
Streptomyces achromogenes
Streptomyces cesalbus
Streptomyces cescaepitosus
Streptomyces cesdiastaticus
Streptomyces cesexfoliatus
Streptomyces fimbriatus
Streptomyces fradiae
Streptomyces fulvissimus
Streptomyces griseoruber
Streptomyces griseus
Streptomyces lavendulae
Streptomyces phaeochromogenes
Streptomyces thermodiastaticus
Streptomyces tubercidicus
Tatlockia
Tatlockia maceachernii
Tatlockia micdadei
Tenacibaculum
Tenacibaculum amylolyticum
Tenacibaculum discolor
Tenacibaculum gallaicum
Tenacibaculum lutimaris
Tenacibaculum mesophilum
Tenacibaculum skagerrakense
Tepidanaerobacter
Tepidanaerobacter syntrophicus
Tepidibacter
Tepidibacter formicigenes
Tepidibacter thalassicus
Thermus
Thermus aquaticus
Thermus filiformis
Thermus thermophilus
Staphylococcus
S. arlettae
S. agnetis
S. aureus
S. auricularis
S. capitis
S. caprae
S. carnosus
S. caseolyticus
S. chromogenes
S. cohnii
S. condimenti
S. delphini
S. devriesei
S. epidermidis
S. equorum
S. felis
S. fleurettii
S. gallinarum
S. haemolyticus
S. hominis
S. hyicus
S. intermedius
S. kloosii
S. leei
S. lentus
S. lugdunensis
S. lutrae
S. lyticans
S. massiliensis
S. microti
S. muscae
S. nepalensis
S. pasteuri
S. petrasii
S. pettenkoferi
S. piscifermentans
S. pseudintermedius
S. pseudolugdunensis
S. pulvereri
S. rostri
S. saccharolyticus
S. saprophyticus
S. schleiferi
S. sciuri
S. simiae
S. simulans
S. stepanovicii
S. succinus
S. vitulinus
S. warneri
S. xylosus
Streptococcus
Streptococcus agalactiae
Streptococcus anginosus
Streptococcus bovis
Streptococcus canis
Streptococcus constellatus
Streptococcus downei
Streptococcus dysgalactiae
Streptococcus equines
Streptococcus faecalis
Streptococcus ferus
Streptococcus infantarius
Streptococcus iniae
Streptococcus intermedius
Streptococcus lactarius
Streptococcus milleri
Streptococcus mitis
Streptococcus mutans
Streptococcus oralis
Streptococcus tigurinus
Streptococcus orisratti
Streptococcus parasanguinis
Streptococcus peroris
Streptococcus pneumoniae
Streptococcus pseudopneumoniae
Streptococcus pyogenes
Streptococcus ratti
Streptococcus salivariu
Streptococcus thermophilus
Streptococcus sanguinis
Streptococcus sobrinus
Streptococcus suis
Streptococcus uberis
Streptococcus vestibularis
Streptococcus viridans
Streptococcus zooepidemicus
Uliginosibacterium
Uliginosibacterium gangwonense
Ulvibacter
Ulvibacter litoralis
Umezawaea
Umezawaea tangerina
Undibacterium
Undibacterium pigrum
Ureaplasma
Ureaplasma urealyticum
Ureibacillus
Ureibacillus composti
Ureibacillus suwonensis
Ureibacillus terrenus
Ureibacillus thermophilus
Ureibacillus thermosphaericus
Vagococcus
Vagococcus carniphilus
Vagococcus elongatus
Vagococcus fessus
Vagococcus fluvialis
Vagococcus lutrae
Vagococcus salmoninarum
Variovorax
Variovorax boronicumulans
Variovorax dokdonensis
Variovorax paradoxus
Variovorax soli
Veillonella
Veillonella atypica
Veillonella caviae
Veillonella criceti
Veillonella dispar
Veillonella montpellierensis
Veillonella parvula
Veillonella ratti
Veillonella rodentium
Venenivibrio
Venenivibrio stagnispumantis
Verminephrobacter
Verminephrobacter eiseniae
Verrucomicrobium
Verrucomicrobium spinosum
Vibrio
Vibrio aerogenes
Vibrio aestuarianus
Vibrio albensis
Vibrio alginolyticus
Vibrio compbellii
Vibrio cholerae
Vibrio cincinnatiensis
Vibrio coralliilyticus
Vibrio cyclitrophicus
Vibrio diazotrophicus
Vibrio fluvialis
Vibrio furnissii
Vibrio gazogenes
Vibrio halioticoli
Vibrio harveyi
Vibrio ichthyoenteri
Vibrio mediterranei
Vibrio metschnikovii
Vibrio mytili
Vibrio natriegens
Vibrio navarrensis
Vibrio nereis
Vibrio nigripulchritudo
Vibrio ordalii
Vibrio orientalis
Vibrio parahaemolyticus
Vibrio pectenicida
Vibrio penaeicida
Vibrio proteolyticus
Vibrio shilonii
Vibrio splendidus
Vibrio tubiashii
Vibrio vulnificus
Virgibacillus
Virgibacillus halodenitrificans
Virgibacillus pantothenticus
Weissella
Weissella cibaria
Weissella confusa
Weissella halotolerans
Weissella hellenica
Weissella kandleri
Weissella koreensis
Weissella minor
Weissella paramesenteroides
Weissella soli
Weissella thailandensis
Weissella viridescens
Williamsia
Williamsia marianensis
Williamsia maris
Williamsia serinedens
Winogradskyella
Winogradskyella thalassocola
Wolbachia
Wolbachia persica
Wolinella
Wolinella succinogenes
Xanthobacter
Xanthobacter agilis
Xanthobacter aminoxidans
Xanthobacter autotrophicus
Xanthobacter flavus
Xanthobacter tagetidis
Xanthobacter viscosus
Xanthomonas
Xanthomonas albilineans
Xanthomonas alfalfae
Xanthomonas arboricola
Xanthomonas axonopodis
Xanthomonas campestris
Xanthomonas citri
Xanthomonas codiaei
Xanthomonas cucurbitae
Xanthomonas euvesicatoria
Xanthomonas fragariae
Xanthomonas fuscans
Xanthomonas gardneri
Xanthomonas hortorum
Xanthomonas hyacinthi
Xanthomonas perforans
Xanthomonas phaseoli
Xanthomonas pisi
Xanthomonas populi
Xanthomonas theicola
Xanthomonas translucens
Xanthomonas vesicatoria
Xylella
Xylella fastidiosa
Xylophilus
Xylophilus ampelinus
Xenophilus
Xenophilus azovorans
Xenorhabdus
Xenorhabdus beddingii
Xenorhabdus bovienii
Xenorhabdus cabanillasii
Xenorhabdus doucetiae
Xenorhabdus griffiniae
Xenorhabdus hominickii
Xenorhabdus koppenhoeferi
Xenorhabdus nematophila
Xenorhabdus poinarii
Xylanibacter
Xylanibacter oryzae
Yangia
Yangia pacifica
Yaniella
Yaniella flava
Yaniella halotolerans
Yeosuana
Yeosuana aromativorans
Yersinia
Yersinia aldovae
Yersinia bercovieri
Yersinia enterocolitica
Yersinia entomophaga
Yersinia frederiksenii
Yersinia intermedia
Yersinia kristensenii
Yersinia mollaretii
Yersinia philomiragia
Yersinia pestis
Yersinia pseudotuberculosis
Yersinia rohdei
Yersinia ruckeri
Yokenella
Yokenella regensburgei
Yonghaparkia
Yonghaparkia alkaliphila
Zavarzinia
Zobellia
Zobellia galactanivorans
Zobellia uliginosa
Zoogloea
Zoogloea ramigera
Zoogloea resiniphila
Zavarzinia compransoris
Zooshikella
Zooshikella ganghwensis
Zunongwangia
Zunongwangia profunda
Zymobacter
Zymobacter palmae
Zymomonas
Zymomonas mobilis
Zymophilus
Zymophilus paucivorans
Zymophilus raffinosivorans
Zobellella
Zobellella denitrificans
Zobellella taiwanensis
Zeaxanthinibacter
Zeaxanthinibacter enoshimensis
Zhihengliuella
Zhihengliuella halotolerans
Xylanibacterium
Xylanibacterium ulmi

1-119. (canceled)

120: An antibacterial composition comprising an engineered mobile genetic element (MGE) that is capable of being mobilized in a first bacterial host cell, wherein the host cell comprises a genome of a first phage, wherein in the host cell the MGE is mobilized using phage proteins of the first phage, wherein the MGE encodes an antibacterial agent or a component thereof, wherein the genome of the first phage comprises a deletion of one or more non-essential genes of a wildtype phage corresponding to the first phage, wherein the first phage is a lytic phage and in the presence of the first phage the mobilization of the MGE causes lysis of the host cell, wherein mobilization of the MGE comprises packaging of copies of the MGE into transduction particles that are capable of transferring the copies into target bacterial cells for antibacterial treatment of the target cells, wherein the antibacterial agent is a guided nuclease system, wherein the antibacterial agent is capable of recognizing and modifying DNA of the target cells.

121: The antibacterial composition of claim 120, wherein the genome of the first phage comprises a deletion of gene C compared to the wildtype phage.

122: The antibacterial composition of claim 120, wherein the genome of the first phage comprises a deletion of DNA between genes C and cox compared to the wildtype phage.

123: The antibacterial composition of claim 120, wherein the genome of the first phage comprises a deletion of gene C and DNA between genes C and cox compared to the wildtype phage.

124: The antibacterial composition of claim 120, wherein the genome of the first phage comprises a deletion of int gene compared to the wildtype phage.

125: The antibacterial composition of claim 120, wherein the genome of the first phage comprises a deletion of int and C genes, or a deletion of int and DNA between genes C and cox.

126: The antibacterial composition of claim 120, wherein the MGE is devoid of a functional integrase gene.

127: The antibacterial composition of claim 124, wherein the MGE is devoid of a functional integrase gene.

128: The antibacterial composition of claim 120, wherein the guided nuclease system is selected from a CRISPR/Cas system, a TALEN system, a meganuclease system and a zinc finger system.

129: The antibacterial composition of claim 128, wherein the guided nuclease system is a CRISPR/Cas system and the MGE encodes: (a) a CRISPR array encoding crRNA, or (b) a nucleic acid encoding a single guide RNA (sgRNA); wherein the crRNA or gRNA is operable with a Cas in a target bacterial cell, wherein the crRNA or gRNA guides the Cas to a target nucleic acid sequence in the target bacterial cell to modify the target nucleic acid sequence.

130: The antibacterial composition of claim 129, wherein the Cas is a Cas encoded by a functional endogenous nucleic acid of the target bacterial cell.

131: The antibacterial composition of claim 129, wherein the Cas is a Cas3, Cas9, Cas13, CasX, CasY or Cpf1.

132: The antibacterial composition of claim 128, wherein the guided nuclease system is a CRISPR/Cas system and the MGE encodes a Cas that is operable in a target bacterial cell to modify a target nucleic acid sequence comprised by the target bacterial cell.

133: The antibacterial composition of claim 128, wherein the guided nuclease system is a CRISPR/Cas system and the MGE encodes one or more of Cascade Cas.

134: The antibacterial composition of claim 133, wherein the MGE further encodes a Cas3 that is operable in a target bacterial cell with the one or more Cascade Cas.

135: The antibacterial composition of claim 120, wherein the MGE is a modified version of a MGE that is naturally found in bacterial cells of the species or strain of the host cell.

136: The antibacterial composition of claim 120, wherein transcription of nucleic acid of the MGE is under the control of a constitutive promoter, for transcription of copies of nucleic acid encoding the antibacterial agent or component thereof in the host cell.

137: The antibacterial composition of claim 120, wherein the host cell is selected from Shigella, Escherichia coli, Serratia, Klebsiella, Yersinia, Pseudomonas, Enterobacter, Staphylococcal, Vibrio, Pseudomonas, Clostridium, Helicobacter and Salmonella cells.

138: The antibacterial composition of claim 120, wherein the target bacterial cells are selected from the group consisting of C. difficile, E. coli, Akkermansia, Enterobacteriacea, Ruminococcus, Faecalibacterium, Firmicutes, Bacteroidetes, Salmonella, Klebsiella, Pseudomonas, Acintenobacter and Streptococcus cells.

139: The antibacterial composition of claim 120, wherein the target bacterial cells are comprised by a microbiota selected from the group consisting of gut microbiota, skin microbiota, oral cavity microbiota, throat microbiota, hair microbiota, armpit microbiota, vaginal microbiota, rectal microbiota, anal microbiota, ocular microbiota, nasal microbiota, tongue microbiota, lung microbiota, liver microbiota, kidney microbiota, genital microbiota, penile microbiota, scrotal microbiota, mammary gland microbiota, ear microbiota, urethra microbiota, labial microbiota, organ microbiota and dental microbiota.

140: The antibacterial composition of claim 139, wherein the target bacterial cells are E. coli cells.

141: The antibacterial composition of claim 139, wherein the target bacterial cells are Pseudomonas cells.

142: The antibacterial composition of claim 120, wherein the composition is capable of knocking-down Clostridium difficile or E. coli bacteria in a gut microbiota of a human or animal.

143: The antibacterial composition of claim 120, wherein the antibacterial agent is capable of recognizing and cutting DNA of the target cells.

144: The antibacterial composition of claim 120, wherein:
a. the target cells are killed by the antibacterial agent;
b. growth or proliferation of the target cells is reduced; or
c. the target cells are sensitized to an antibiotic, whereby the antibiotic is toxic to the target cells.
